SEQUENCE LISTING (30 sequences)

SEQ ID NO: 1 - protein, 282 aa, Homo sapiens <s300> AAA63170.1, GenBank 1995-03-06 [sequence omitted]

SEQ ID NO: 2 - protein, 282 aa, Homo sapiens <s300> CAC12813.1, GenBank 2008-10-07 [sequence omitted]

SEQ ID NO: 3 - protein, 302 aa, Homo sapiens <s300> AAB00495.1, GenBank 1996-05-21 [sequence omitted]

SEQ ID NO: 4 - protein, 282 aa, Bos Taurus <s300> P00639, UniProtKB/Swiss-Prot 1998-12-15
MRGTRLMGLLLALAGLLQLGLSLKIAAFNIRTFGETKMSNATLASYIVRIVRRYDIVLIQEVRDSHLVAVGKLLDYLNQDDPNTYHYVVSEPLGRNSYKERYLFLFRPNKVSVLDTYQYDDGCESCGNDSFSREPAVVKFSSHSTKVKEFAIVALHSAPSDAVAEINSLYDVYLDVQQKWHLNDVMLMGDFNADCSYVTSSQWSSIRLRTSSTFQWLIPDSADTTATSTNCAYDRIVVAGSLLQSSVVPGSAAPFDFQAAYGLSNEMALAISDHYPVEVTLT

SEQ ID NO: 5 - protein, 15 aa, Artificial Sequence
  Description of Artificial Sequence Synthetic DNase I motif with positively-charged basic amino acids at positions Q9 and E13 conferring hyperactivity
  MOD_RES (6)..(6): Any basic amino acid (His, Arg, Lys, Asn or Gln)
  MOD_RES (7)..(7): Ser or Thr
  MOD_RES (10)..(10): Any basic amino acid (His, Arg, Lys, Asn or Gln)
AAFNIXXFGXTKMSN

SEQ ID NO: 6 - protein, 11 aa, Artificial Sequence
  Description of Artificial Sequence Synthetic DNase I motif with positively-charged basic amino acid at position N74 conferring heat lability
  MOD_RES (7)..(7): Any basic amino acid (His, Arg, Lys, Asn or Gln)
  MOD_RES (8)..(8): Ser, Thr or Asn
SEPLGRXXYKE

SEQ ID NO: 7 - protein, 7 aa, Artificial Sequence
  Description of Artificial Sequence Synthetic DNase I motif with Leu130 conferring heat lability
  MOD_RES (5)..(5): Ala or Pro
FALVXLH

SEQ ID NO: 8 - protein, 20 aa, Artificial Sequence
  Description of Artificial Sequence Synthetic DNase I motif with Ser-205 conferring heat lability
  MOD_RES (5)..(5): Ala, Ser or Val
  MOD_RES (7)..(7): Thr, Ser or Lys
  MOD_RES (8)..(8): Ser or Pro
  MOD_RES (10)..(10): Asn or His
ADTTXSXXTXCAYDRIVVAG

SEQ ID NO: 9 - protein, 260 aa, Homo sapiens
  human DNase I (phosphodiesterase)
LKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCGNDTFNREPAIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLK

SEQ ID NO: 10 - protein, 260 aa, Bos taurus
  bovine DNase I (phosphodiesterase)
LKIAAFNIRTFGETKMSNATLASYIVRIVRRYDIVLIQEVRDSHLVAVGKLLDYLNQDDPNTYHYVVSEPLGRNSYKERYLFLFRPNKVSVLDTYQYDDGCESCGNDSFSREPAVVKFSSHSTKVKEFAIVALHSAPSDAVAEINSLYDVYLDVQQKWHLNDVMLMGDFNADCSYVTSSQWSSIRLRTSSTFQWLIPDSADTTATSTNCAYDRIVVAGSLLQSSVVPGSAAPFDFQAAYGLSNEMALAISDHYPVEVTLT

SEQ ID NO: 11 - protein, 260 aa, Ovis aries
  ovine DNase I (phosphodiesterase)
LKIAAFNIRTFGETKMSNATLSSYIVRILRRYDIALIQEVRDSHLVAVGKLLDDLNQDDPNSYHYVVSEPLGRNSYKERYLFVFRPNKVSVLDTYQYDDGCESCGNDSFSREPAVVKFSSPSTKVKAFAIVPLHSAPSDAVAEINSLYDVYLDVQQKWDLNDIMLMGDFNADCSYVTSSQWSSIRLRTSSTFQWLIPDSADTTATSTNCAYDRIVVAGSLLQSSVVPGSAVPFDFQAAYGLSNEMALAISDHYPVEVTLT

SEQ ID NO: 12 - protein, 260 aa, Equus caballus
  equine DNase I (phosphodiesterase)
LRIAAFNIRTFGETKMSNDTLSNYIVQILNRYDIALIQEVRDSHLTAVGKLLDRLNQDDPNTYHFVVSEPLGRNNYKERYLFVFRPDQVSLLDSYQYNDGCEPCGNDTFSREPAIVKFSSPFTQVKEFAIVPLHAAPSDALAEIDSLYDVYLDVQQKWDMEDIMLMGDFNAGCSYVTSSQWPSIRLRRNPAFWWLIPDTADTTVKSTHCAYDRIVVAGTLLQEAVVPDSAVPFDFQAAYGLNDQTAEAISDHYPVEVTLM

SEQ ID NO: 13 - protein, 262 aa, Canis familiaris
  canine DNase I (phosphodiesterase)
LRMAAFNIRTFGETKMSNATLSKYIVQILSRYDVAVVQEVRDSHLTAVGKLLDTLNQDDPNAYHYVVSEPLGRSSYKERYLFLFRPDRVSVLDSYQYDDGCEPCGNDTFSREPAIVRFHSPLTEVKEFAVVPLHAAPLDAVAEIDALYDVYLDVQHKWDLEDIVLMGDFNAGCSYVAASQWSSIRLRTNPAFQWLIPDTADTTSTSTHCAYDRIVVAGSQLQHAVVPESAAPFNFQVAYGLSSQLAQAISDHYPVEVTLKRA

SEQ ID NO: 14 - protein, 262 aa, Sus scrofa
  porcine DNase I (phosphodiesterase)
LRIAAFNIRTFGETKMSNATLSNYIVRILSRYDIALIQEVRDSHLTAVGKLLNELNQDDPNNYHHVVSEPLGRSTYKERYLFVFRPDQVSVLDSYLYDDGCEPCGNDTFNREPSVVKFSSPSTQVKEFAIVPLHAAPSDAAAEIDSLYDVYLNVRQKWDLEDIMLMGDFNAGCSYVTTSHWSSIRLRESPPFQWLIPDTADTTVSSTHCAYDRIVVAGPLLQRAVVPDSAAPFDFQAAFGLSEQTALAISDHYPVEVTLKRA

SEQ ID NO: 15 - protein, 260 aa, Oryctolagus cuniculus
  rabbit DNase I (phosphodiesterase)
LKIAAFNIRSFGETKMSNATLTSYIVRILQRYDIALIQEVRDSHLTAVGKLLDKLNEKAADTYRFVASEPLGRRTYKERYLFVYRPDQVSVLDSYYYDDGCEPCGTDTFSREPAVVRFSSPSTKVREFAIVPLHSAPEDAVAEIDALYDVYLDVQKKWGLQDVMLMGDFNADYSYVTSSQWSSIRLRTNPAFKWLIPDTADTTATSTNCAYDRIVVAGPLLQDAVVPNSAAPFNFQAAYGLSNQLAQAISDHYPVEVTLA

SEQ ID NO: 16 - protein, 262 aa, Mus musculus
  mouse DNase I (phosphodiesterase)
LRIAAFNIRTFGETKMSNATLSVYFVKILSRYDIAVIQEVRDSHLVAVGKLLDELNRDKPDTYRYVVSEPLGRKSYKEQYLFVYRPDQVSILDSYQYDDGCEPCGNDTFSREPAIVKFFSPYTEVQEFAIVPLHAAPTEAVSEIDALYDVYLDVWQKWGLEDIMFMGDFNAGCSYVTSSQWSSIRLRTSPIFQWLIPDSADTTVTSTHCAYDRIVVAGALLQAAVVPNSAVPFDFQAEYGLSNQLAEAISDHYPVEVTLRKI

SEQ ID NO: 17 - protein, 262 aa, Rattus norvegicus
  rat DNase I (phosphodiesterase)
LRIAAFNIRTFGDTKMSNATLSSYIVKILSRYDIAVVQEVRDTHLVAVGKLLDELNRDIPDNYRYIISEPLGRKSYKEQYLFVYRPSQVSVLDSYHYDDGCEPCGNDTFSREPAIVKFFSPYTEVREFAIVPLHSAPTEAVSEIDALYDVYLDVRQKWGLEDIMFMGDFNAGCSYVTSSQWSSIRLRTSPIFQWLIPDSADTTATSTHCAYDRIVVAGALLQAAVVPSSAVPFDFQAEYRLTNQMAEAISDHYPVEVTLRKT

SEQ ID NO: 18 - protein, 262 aa, Gallus gallus
  chicken DNase I (phosphodiesterase)
LRISAFNIRTFGDSKMSNQTVAGFIVSILVQYDITLVQEVRDADLSSVKKLVSQLNSASSYPYSFLSSIPLGRNSYKEQYVFIYRSDIVSVLESYYYDDGCESCGTDIFSREPFIVKFSSPTTQLDEFVIVPLHAEPSSAPAEINALTDVYTDVINKWETNNIFFMGDFNADCSYVTAEQWPSIRLRSLSSCEWLIPDSADTTVTSTDCAYDRIVACGSALRQAVEYGSATVNNFQETLRIQNKDALAISDHFPVEVTLKAR

SEQ ID NO: 19 - protein, 262 aa, Elaphe quadrivirgata
  snake DNase I (phosphodiesterase)
LRIGAFNIRAFGDKKLSNQTISSSIVRILTTYDLVLIQEVRDADLSAVKKLMQLVSGASPDPFGYLISKPLGHNSYKEQYLFVYRQDRVSPVESYYYDDGCEPCGNGTFSREPFIVKFAVPQAAVEELVLVPLHAAPEAAVTEIDSLYDVYQDVKDRWGVTDALLLGDFNADCNYVQAEDWPSIRLRSSKDFQWLIPDTADTTVTNTICAYDRIVAVGSKLRESILPATAKVDNFQKTLKLSSKDALAVSDHFPVEVTLKST

SEQ ID NO: 20 - protein, 331 aa, Xenopus laevis
  African clawed frog DNase I (phosphodiesterase)
FKIASFNIQRFSMTKVDDPVVLELLIRILSRYEIIAIEEVMNADNTAIISLVKELSLATKLNYNVLISDHLGRSSYREKYAYVYREDIVKPTEWYHFDDGCENCGTDSFIREPFVARFTSLTTVVKDFALISIHTSPDYAIMEVDALYDAWVDAKQRLKMENILILGDYNAACSYVASRHWPIIRLRHVEELVWLIGDKEDTTVSTNTNCAYDRMVAGGEELQRGIVPDTAKAFNYHVAYDLTYEMAKAVSDHYPVEVELYDDVFYSGQCFEPSASTGISGGLSLNGPCTCEGVDFSSCRGRCGASGKTYPCNCNASCTNCCVDYTSSCKL

SEQ ID NO: 21 - protein, 262 aa, Anguilla japonica
  Japanese eel DNase I (phosphodiesterase)
LFIGAFNIRSFGDKKASNATLVDIIVKIVHMYDILLIQEVRDSDLSATKKLMQNVNGGSSPHKYKYIVSEPLGRNTYQERYLYLYREDSVSVVKNFTYDDGAEASGTDTFNREPFVVMFSSPHTRVPEFALVPQHTSPDEAVKEIDALYDVIVDIRARWNTDNIILLGDFNAGCNYVAGSDWQQIRLYTDKSFHWLIPDSADTTVSHTNCPYDRIVATTTMMEAVVPHSASVYDYMTSLKLKLDMALAVSDHFPVEVQLFGP

SEQ ID NO: 22 - protein, 261 aa, Artificial Sequence
  Description of Artificial Sequence Synthetic equine DNase I “KS” mutant having two hyperactive (R13, K74) and two heat-labile (L130, S-205 insertion) mutations
LRIAAFNIRTFGRTKMSNDTLSNYIVQILNRYDIALIQEVRDSHLTAVGKLLDRLNQDDPNTYHFVVSEPLGRKNYKERYLFVFRPDQVSLLDSYQYNDGCEPCGNDTFSREPAIVKFSSPFTQVKEFALVPLHAAPSDALAEIDSLYDVYLDVQQKWDMEDIMLMGDFNAGCSYVTSSQWPSIRLRRNPAFWWLIPDTADTTVSKSTHCAYDRIVVAGTLLQEAVVPDSAVPFDFQAAYGLNDQTAEAISDHYPVEVTLM

SEQ ID NO: 23 - protein, 260 aa, Artificial Sequence
  Description of Artificial Sequence Synthetic equine DNase I “K” mutant having two hyperactive (R13, K74) and one heat-labile (L130) mutation
LRIAAFNIRTFGRTKMSNDTLSNYIVQILNRYDIALIQEVRDSHLTAVGKLLDRLNQDDPNTYHFVVSEPLGRKNYKERYLFVFRPDQVSLLDSYQYNDGCEPCGNDTFSREPAIVKFSSPFTQVKEFALVPLHAAPSDALAEIDSLYDVYLDVQQKWDMEDIMLMGDFNAGCSYVTSSQWPSIRLRRNPAFWWLIPDTADTTVKSTHCAYDRIVVAGTLLQEAVVPDSAVPFDFQAAYGLNDQTAEAISDHYPVEVTLM

SEQ ID NO: 24 - protein, 15 aa, Artificial Sequence
  Description of Artificial Sequence Synthetic DNase I motif with positively-charged basic amino acids at positions Q9 and E13 conferring hyperactivity
  MOD_RES (7)..(7): Ser or Thr
AAFNIRXFGRTKMSN

SEQ ID NO: 25 - protein, 11 aa, Artificial Sequence
  Description of Artificial Sequence Synthetic DNase I motif with positively-charged basic amino acid at position N74 conferring heat lability
  MOD_RES (8)..(8): Ser, Thr or Asn
SEPLGRKXYKE

SEQ ID NO: 26 - protein, 262 aa, Artificial Sequence
  Description of Artificial Sequence Synthetic DNase I consensus sequence
  MOD_RES (27)..(27): Any amino acid
  MOD_RES (54)..(54): Any amino acid
  MOD_RES (74)..(74): Any amino acid
  MOD_RES (122)..(122): Any amino acid
  MOD_RES (124)..(124): Any amino acid
  MOD_RES (138)..(138): Any amino acid
  MOD_RES (159)..(159): Any amino acid
  MOD_RES (191)..(191): Any amino acid
  MOD_RES (219)..(219): Any amino acid
  MOD_RES (223)..(223): Any amino acid
  MOD_RES (228)..(228): Any amino acid
  MOD_RES (231)..(231): Any amino acid
  MOD_RES (245)..(245): Any amino acid
  MOD_RES (247)..(247): Any amino acid
  MOD_RES (260)..(260): Any amino acid
  MOD_RES (261)..(261): Any amino acid
  MOD_RES (262)..(262): Any amino acid
LRIAAFNIRTFGETKMSNATLSSYIVXILSRYDIALIQEVRDSHLTAVGKLLDXLNQDDPNTYHYVVSEPLGRXSYKERYLFVFRPDQVSVLDSYQYDDGCEPCGNDTFSREPAIVKFSSPXTXVKEFAIVPLHAAPXDAVAEIDALYDVYLDVQQKWXLEDIMLMGDFNAGCSYVTSSQWSSIRLRTSPXFQWLIPDSADTTATSTHCAYDRIVVAGXLLQXAVVPXSAXPFDFQAAYGLSNQXAXAISDHYPVEVTLXXX

SEQ ID NO: 27 - protein, 6 aa, Artificial Sequence
  Description of Artificial Sequence Synthetic 6xHis tag
HHHHHH

SEQ ID NO: 28 - protein, 20 aa, Artificial Sequence
  Description of Artificial Sequence Synthetic Peptide
  MISC_FEATURE (1)...(20): This sequence may encompass 1-10 Gly Serrepeating units
GSGSGSGSGSGSGSGSGSGS

SEQ ID NO: 29 - protein, 30 aa, Artificial Sequence
  Description of Artificial Sequence Synthetic polypeptide
  MISC_FEATURE (1)...(20): This sequence may encompass 1-10 Gly Gly Ser repeating units
GGSGGSGGSGGSGGSGGSGGSGGSGGSGGS

SEQ ID NO: 30 - protein, 40 aa, Artificial Sequence
  Description of Artificial Sequence Synthetic polypeptide
  MISC_FEATURE (1)...(20): This sequence may encompass 1-10 Gly Gly Gly Ser repeating units
GGGSGGGSGGGSGGGSGGGSGGGSGGGSGGGSGGGSGGGS

(19) United States
(12) Patent Application Publication
(10) Pub. No.: US 2021/0369818 A1
(43) Pub. Date: Dec. 2, 2021

(54) HUMAN DNASE FOR LUNG DISEASE

(71) Applicant: GRO BIOSCIENCES INC., Cambridge, MA (US)

(72) Inventor: Daniel J. Mandell, Brookline, MA (US)

(21) Appl. No.: 17/323,383

(22) Filed: May 18, 2021

Related U.S. Application Data
(63) Continuation of application No. PCT/US2019/062199, filed on Nov. 19, 2019.
(60) Provisional application No. 62/769,258, filed on Nov. 19, 2018.

(51) Int. Cl. A61K 38/46; A61K 45/06; A61K 38/06

(52) U.S. Cl. CPC A61K 38/465; C12Y 301/21001; A61K 38/063; A61K 45/06

(57) ABSTRACT

Provided herein are pharmaceutical compositions for treating human lung diseases comprising a stabilized DNase I polypeptide containing a non-standard amino acid, a functional fragment thereof, or a variant thereof that maintains enzymatic activity even under harsh conditions, such as reducing environments. The present disclosure also relates to methods of using the composition for the treatment of human of lung diseases or for the disruption of biofilms.

### CROSS-REFERENCE

This application is a continuation of International Application No. PCT/US2019/062199, filed on Nov. 19, 2019, which claims benefit of U.S. Provisional Application No. 62/769,258, filed Nov. 19, 2018, each of which is entirely incorporated herein by reference for all purposes.

### BACKGROUND

Cystic Fibrosis (CF) is one of the common human lung diseases that cause persistent lung infections and is often evidenced by an overproduction by mucus in the airways. In patients with CF, a defective gene causes a buildup of thick mucus in the lungs that not only blocks the airways but also traps bacteria, which leads to persistent, chronic infection and eventually respiratory failure. A biofilm with an extracellular polymeric substance matrix is commonly observed in the CF sputum. The matrix is found to contain a combination of secreted polysaccharide, extracellular DNA (eDNA), and extracellular structured proteins, which creates a biofilm structure that is resistant to external environment and harbors bacteria. Mucus thinners such mucolytics may be used to treat CF, and other lung diseases where mucus overproduction are observed, in order to loosen the mucus and clear the airways. One of the effective mucus thinners is dornase alfa, a recombinant human deoxyribonuclease I (DNase I) polypeptide that selectively cleaves the eDNA.

However, like many other enzymes, the activity of dornase alfa depends on the environment in which it catalyzes the reactions, for example, buffers with reducing conditions. Often, enzymes rely on certain structural features to maintain their activity and these structural features may be compromised by certain conditions. For example, in reducing environments such as found in human lungs in conditions related to treatment of pulmonary disease, the intermolecular and/or intramolecular disulfide bridges of enzymes may be reduced, leading to structural changes in the enzyme and ultimately, reduction or loss of activity. Thus, there remains a need for DNase I polypeptide containing pharmaceutical compositions and methods of effectively disrupting the biofilm that are compatible with human lung environment.

### SUMMARY

Provided herein are pharmaceutical compositions for treating human lung diseases comprising a stabilized DNase I polypeptide containing a non-standard amino acid, a functional fragment thereof, or a variant thereof that maintains enzymatic activity even under harsh conditions, such as reducing environments. Provided herein are methods of using the composition for the treatment of human of lung diseases or for the disruption of biofilms. Further provided herein are kits that comprise the pharmaceutical compositions.

In one aspect, provided herein is a method of treating a human subject with a lung disease comprising administering to the human subject a stabilized deoxyribonuclease I (DNase I) polypeptide comprising one or more non-standard amino acids, a functional fragment thereof, or a variant thereof.

In some embodiments, the method comprises administering to the subject one or more antibiotics.

In some embodiments, the method comprises administering to the subject glutathione (GSH).

In some embodiments, an amount of the GSH administered is an amount effective to enhance an efficiency of the one or more antibiotics.

In some embodiments, the method comprises administering to the human subject via inhalation a pharmaceutical composition comprising the stabilized DNase I polypeptide, functional fragment thereof, or variant thereof.

In some embodiments, the pharmaceutical composition is a dispersible powder or an aerosolable solution or suspension.

In some embodiments, the pharmaceutical composition is delivered or administered via a nebulizer, a pressurized metered-dose inhaler, or a dry powder inhaler.

In some embodiments, the pharmaceutical composition is delivered as an aerosol.

In some embodiments, the aerosol has a predetermined mass medial aerodynamic diameter (MMAD) from about 1 μm to 6 μm, 2 μm to 6 μm, 2 μm to 5 μm, or 2 μm to 4 μm.

In some embodiments, the pharmaceutical composition further comprises at least one pharmaceutically acceptable carrier, excipient, or additive.

In some embodiments, the pharmaceutical composition comprises saline or sterile water.

In some embodiments, the stabilized DNase polypeptide, functional fragment thereof, or variant thereof is present in the pharmaceutical composition or in the aerosol at a concentration of about 0.1 mg/ml to 10 mg/ml, 0.5 mg/ml to 2 mg/ml, or 0.75 mg/ml to 1.25 mg/ml.

In some embodiments, about from 0.1-20 ml, 0.1-10 ml, 0.5-10 ml, 0.5-5 ml, 0.5-4 ml, 0.5-3 ml, 0.75-3 ml, 0.75-2.5 ml, 0.75-2 ml, 0.75-1.5 ml, or 0.75-1.25 ml is administered.

In some embodiments, the aerosol is delivered to the human subject within 30, 20, 15, or 10 minutes for each administration.

In some embodiments, the stabilized DNase polypeptide, functional fragment thereof, or variant thereof is administered 1, 2, 3, 4, 5, or more times a day or 1, 2, 3, 4, 5, or more times a week.

In some embodiments, the stabilized DNase polypeptide, functional fragment thereof, or variant thereof, the GSH, and the one or more antibiotics are administered simultaneously or sequentially.

In some embodiments, the one or more antibiotics are selected from the group of classes consisting of: Penicillins, Tetracyclines, Cephalosporins, Quinolones, Lincomycins, Macrolides, Sulfonamides, Glycopeptides, Aminoglycosides and Carbapenems.

In some embodiments, the one or more antibiotics comprise amoxicillin, doxycycline, cephalexin, ciprofloxacin, clindamycin, metronidazole, azithromycin, sulfamethoxazole/trimethoprim, amoxicillin/clavulanate, levofloxacin, cefepime, tazobactam/piperacillin, meropenem, amikacin, gentamicin, aztreonam, colistin, or tobramycin.

In some embodiments, the one or more antibiotics comprise an inhalable antibiotic.

In some embodiments, the one or more antibiotics comprise aztreonam, colistin, tobramycin, or any combination thereof.

In one aspect, provided herein is a pharmaceutical composition comprising a stabilized DNase I polypeptide comprising one or more non-standard amino acids, a functional fragment thereof, or a variant thereof; and at least one pharmaceutically acceptable carrier, excipient, or additive.

In some embodiments, the pharmaceutical composition is for treating a human subject with lung disease.

In some embodiments, the pharmaceutical composition comprises one or more antibiotics.

In some embodiments, the pharmaceutical composition comprises GSH.

In some embodiments, the GSH is present in the pharmaceutical composition in an amount effective to enhance an efficiency of the one or more antibiotics.

In some embodiments, the pharmaceutical composition is in a powder form.

In some embodiments, the pharmaceutical composition is a dispersible powder.

In some embodiments, the pharmaceutical composition is prepared by a method comprising spray-drying a liquid composition comprising the stabilized DNase I polypeptide, functional fragment thereof, or variant thereof and the at least one pharmaceutically acceptable carrier, excipient, or additive, and collecting the spray-dried product of step (a) as a dispersible powder containing the stabilized DNase I polypeptide, functional fragment thereof, or variant thereof.

In some embodiments, the pharmaceutical composition is prepared by a method further comprising combining the dispersible powder with GSH.

In some embodiments, the pharmaceutical composition is prepared by a method further comprising combining the dispersible powder with the one or more antibiotics.

In some embodiments, the liquid composition has a concentration of the stabilized DNase I polypeptide, functional fragment thereof, or variant thereof of about from 1 mg/ml to 80 mg/ml.

In some embodiments, the stabilized DNase I polypeptide, functional fragment thereof, or variant thereof is present in the dispersible powder in an amount of about 1% to 100% by weight.

In some embodiments, the pharmaceutical composition is a liquid form.

In some embodiments, the pharmaceutical composition is an aerosolable solution or suspension.

In some embodiments, the aerosolable solution or suspension has a concentration of the stabilized DNase I polypeptide, functional fragment thereof, or variant thereof of about from 0.1 mg/ml to 20 mg/ml, 0.1 mg/ml to 10 mg/ml, 0.5 mg/ml to 5, 0.5 mg/ml to 2 mg/ml, or 0.75 mg/ml to 1.25 mg/ml.

In some embodiments, the pharmaceutical composition is a dosage form and comprises 0.1-20 ml, 0.1-10 ml, 0.5-10 ml, 0.5-5 ml, 0.5-4 ml, 0.5-3 ml, 0.75-3 ml, 0.75-2.5 ml, 0.75-2 ml, 0.75-1.5 ml, or 0.75-1.25 ml.

In some embodiments, the pharmaceutical composition is a dosage form and the stabilized DNase I polypeptide, functional fragment thereof, or variant thereof is present in the dosage form at an amount of about 0.1 mg to 20 mg, 0.1 mg to 10 mg, 0.1 mg to 5 mg, 0.5 mg to 4 mg, 0.5 mg to 3 mg, 0.5 mg to 2.5 mg, 0.5 mg to 2 mg, 0.5 mg to 1.5 mg, 0.75 mg to 1.5 mg, 0.75 mg to 1.25 mg, or 1.5 mg to 2.5 mg.

In some embodiments, the aerosolable solution or suspension has a concentration of the GSH of about from 1 mg/ml to 800 mg/ml, 50 mg/ml to 400 mg/ml, 75 mg/ml to 300 mg/ml, 100 mg/ml to 200 mg/ml, 125 mg/ml to 175 mg/ml, 1 mg/ml to 200 mg/ml, 1 mg/ml to 100 mg/ml, 1 mg/ml to 50 mg/ml, or 1 mg/ml to 20 mg/ml.

In some embodiments, the pharmaceutical composition is a dosage form and has an amount of the GSH of at least about 1 mg, 10 mg, 20 mg, 30 mg, 40 mg, 50 mg, 60 mg, 70 mg, 80 mg, 90 mg, 100 mg, 150 mg, 200 mg, 300 mg, 400 mg, 500 mg, 600 mg, 700 mg, 800 mg, or 1000 mg.

In some embodiments, a weight ratio of the GSH and the stabilized DNase I polypeptide, functional fragment thereof, or variant thereof present in the pharmaceutical composition for an administration is at least 6:1, 25:1, 50:1, 100:1, 200:1, 300:1, 400:1, 500:1, 600:1, 800:1, 6000:1, or 60000:1.

In some embodiments, the at least one pharmaceutically acceptable carrier, excipient, or additive comprises saline or sterile water.

In some embodiments, the one or more antibiotics are selected from the classes consisting of: Penicillins, Tetracyclines, Cephalosporins, Quinolones, Lincomycins, Macrolides, Sulfonamides, Glycopeptides, Aminoglycosides and Carbapenems.

In some embodiments, the one or more antibiotics comprise amoxicillin, doxycycline, cephalexin, ciprofloxacin, clindamycin, metronidazole, azithromycin, sulfamethoxazole/trimethoprim, amoxicillin/clavulanate, levofloxacin, cefepime, tazobactam/piperacillin, meropenem, amikacin, gentamicin, aztreonam, colistin, or tobramycin.

In some embodiments, the one or more antibiotics comprise an inhalable antibiotic.

In some embodiments, the one or more antibiotics comprise aztreonam, colistin, tobramycin, or any combination thereof.

In another aspect, provided herein is a method of disrupting a biofilm comprising contacting a stabilized DNase I polypeptide comprising one or more non-standard amino acids, a functional fragment thereof, or a variant thereof, to the biofilm.

In some embodiments, the biofilm is on an environmental surface or on the surface of a medical device.

In some embodiments, the biofilm is in the respiratory tract of a human subject having a lung disease.

In some embodiments, the method further comprises contacting one or more antibiotics to the biofilm.

In some embodiments, the method further comprises contacting GSH to the biofilm.

In some embodiments, the GSH is in an amount effective to enhance an efficiency of the one or more antibiotics.

In some embodiments, the lung disease is a pulmonary disease or condition.

In some embodiments, the pulmonary disease or condition is an acute or chronic bronchopulmonary disease, atelectasis due to tracheal or bronchial impaction, or complications of tracheostomy.

In some embodiments, the pulmonary disease or condition is infectious pneumonia, bronchitis or tracheobronchitis, bronchiectasis, cystic fibrosis (CF), asthma, tuberculosis (TB), or fungal infections.

In some embodiments, the lung disease is CF.

In one aspect, provided herein is a kit comprising a first component comprising a stabilized DNase I polypeptide comprising one or more non-standard amino acids, a functional fragment thereof, or a variant thereof, and a second component comprising GSH.

In another aspect, provided herein is a kit comprising a first component comprising a stabilized DNase I polypeptide comprising one or more non-standard amino acids, a functional fragment thereof, or a variant thereof, and a second component comprising one or more antibiotics.

In some embodiments, the first component and the second component are packaged separately in the kit.

In some embodiments, at least part of the first component and part of the second component are combined in the kit.

In some embodiments, the kit further comprises a third component comprising one or more antibiotics.

In some embodiments, the kit further comprises a third component comprising GSH.

In some embodiments, the first component, the second component, the third component, or a combination thereof is suitable to be administered by inhalation.

In some embodiments, the stabilized DNase I polypeptide has a higher endonuclease activity for a DNA substrate in an environment than an endonuclease activity for the DNA substrate of a corresponding DNase I polypeptide, functional fragment thereof, or variant thereof that does not comprise the one or more non-standard amino acids.

In some embodiments, the stabilized DNase I polypeptide does not destabilize in an environment that a corresponding DNase I polypeptide, functional fragment thereof, or variant thereof that does not comprise the one or more non-standard amino acids does destabilize.

In some embodiments, the stabilized DNase I polypeptide has a melting temperature (Tm) that is at least 5° C. higher than a Tm of a corresponding DNase I polypeptide, functional fragment thereof, or variant thereof that does not comprise the one or more non-standard amino acids.

In some embodiments, at least one, two, three, four or more of the one or more non-standard amino acids is selenocysteine.

In some embodiments, at least two of the one or more non-standard amino acids are directly linked by a bond.

In some embodiments, the bond is a diselenide bond.

In some embodiments, the bond is a selenyl-sulfhydryl bond between a cysteine and a selenocysteine.

In some embodiments, the stabilized DNase I polypeptide, functional fragment thereof, or variant thereof, has a half-life that is at least 1.1 fold higher than a half-life of a corresponding DNase I polypeptide, functional fragment thereof, or variant thereof that does not comprise the one or more non-standard amino acids.

In some embodiments, the stabilized DNase I polypeptide, functional fragment thereof, or variant thereof, has at least 70%, 75%, 80%, 85%, 90%, 95% sequence identity to SEQ ID NO:1, 2, or 3.

In some embodiments, the stabilized DNase I polypeptide, functional fragment thereof, or variant thereof, comprises a sequence with at least 70%, 75%, 80%, 85%, 90%, 95% sequence identity to at least 25, 50, 75, 100, 125, 150, 175, 200, 225, 250, 260, 270, 280, or 282 contiguous amino acids of SEQ ID NO: 1.

In some embodiments, the one or more non-standard amino acids is at: position 123 of SEQ ID NO:1 or 2, position 126 of SEQ ID NO:1 or 2, position 195 of SEQ ID NO:1 or 2, or position 187 of SEQ ID NO: 3, or position 231 of SEQ ID NO:1 or 2, or position 224 of SEQ ID NO: 3.

In some embodiments, a non-standard amino acid at position 123 of SEQ ID NO: 1 or 2 is directly linked by a bond to a non-standard amino acid at position 126.

In some embodiments, a non-standard amino acid at position 195 of SEQ ID NO: 1 or 2 is directly linked by a bond to a non-standard amino acid at position 231.

In some embodiments, a non-standard amino acid at position 187 of SEQ ID NO: 3 is directly linked by a bond to a non-standard amino acid at position 224.

In some embodiments, the method comprises expressing an amino acid sequence of the stabilized DNase I polypeptide.

In some embodiments, the expressing comprises expressing in a cell or in vitro.

In some embodiments, the cell is a bacterial cell.

In some embodiments, the cell is a genomically recoded cell.

In some embodiments, the cell comprises a reassigned codon recognized by a stabilizing non-standard amino acid tRNA comprising an anticodon corresponding to the reassigned codon.

In some embodiments, the amino acid sequence of the DNase I polypeptide, functional fragment thereof, or variant thereof is encoded by a polynucleotide sequence comprising at least one codon of a natural amino acid that has been replaced by the reassigned codon.

In some embodiments, the stabilizing non-standard amino acid tRNA is a selenocysteine tRNA.

In some embodiments, the method comprises culturing the cell under conditions in which the amino acid sequence of the DNase I polypeptide is expressed.

In some embodiments, the reassigned codon is UAG, UAA, UGA, or a combination thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1 shows a protein structure of human DNase.

FIG. 2 shows an SDS-PAGE gel demonstrating the purity of recombinant seleno-DNase at various purification steps.

FIG. 3A-B shows the activity of GRO seleno-DNase versus recombinant human DNase at various concentrations of gluthione. FIG. 3A shows the activity at 0 mM glutathione. FIG. 3B shows the activity at 400 mM glutathione.

### DETAILED DESCRIPTION

The following descriptions illustrate embodiments of the present disclosure in detail. It is to be understood that this present disclosure is not limited to the particular embodiments described herein and as such can vary. Those of skill in the art will recognize that there are numerous variations and modifications of this present disclosure, which are encompassed within its scope.

Although various features of the present disclosure may be described in the context of a single embodiment, the features may also be provided separately or in any suitable combination. Conversely, although the present disclosure may be described herein in the context of separate embodiments for clarity, the present disclosure may also be implemented in a single embodiment.

The section headings used herein are for organizational purposes only and are not to be construed as limiting the subject matter described.

All terms are intended to be understood as they would be understood by a person skilled in the art. Unless defined otherwise, all technical and scientific terms used herein have the same meaning as commonly understood by one of ordinary skill in the art to which the disclosure pertains.

The following definitions supplement those in the art and are directed to the current application and are not to be imputed to any related or unrelated case, e.g., to any commonly owned patent or application. Although any methods and materials similar or equivalent to those described herein can be used in the practice for testing of the present disclosure, the preferred materials and methods are described herein. Accordingly, the terminology used herein is for the purpose of describing particular embodiments only, and is not intended to be limiting.

### I. Definitions

The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The term “endogenous” refers to any material from or produced inside an organism, cell, tissue or system.

The term “exogenous” refers to any material introduced from or produced outside an organism, cell, tissue or system.

The term “expression” refers to the transcription and/or translation of a particular nucleotide sequence driven by a promoter.

The term “homologous” or “identity” refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous or identical at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

The term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

The term “operably linked” or “transcriptional control” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Operably linked DNA sequences can be contiguous with each other and, e.g., where necessary to join two protein coding regions, are in the same reading frame.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The term “amino acid” as used herein refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, gamma-carboxyglutamate, and O-phosphoserine. The term “amino acid analogs” as used herein refers to compounds that have the same basic chemical structure as a naturally occurring amino acid, i.e., an alpha carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. The term “amino acid mimetics” as used herein refers to chemical compounds that have a structure that is different from the general chemical structure of an amino acid, but that functions in a manner similar to a naturally occurring amino acid.

The term “non-standard amino acid” refers to any amino acid other than the 20 standard amino acids (alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine). Selenocysteine is a non-standard amino acid (NSAA).

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

The term “promoter” refers to a DNA sequence recognized by the transcription machinery of the cell, or introduced synthetic machinery, required to initiate the specific transcription of a polynucleotide sequence.

The term “constitutive” promoter refers to a nucleotide sequence which, when operably linked with a polynucleotide which encodes or specifies a gene product, causes the gene product to be produced in a cell under most or all physiological conditions of the cell.

The term “inducible” promoter refers to a nucleotide sequence which, when operably linked with a polynucleotide which encodes or specifies a gene product, causes the gene product to be produced in a cell substantially only when an inducer which corresponds to the promoter is present in the cell.

The term “transfected” or “transformed” or “transduced” refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. As another example, a range such as 95-99% identity, includes something with 95%, 96%, 97%, 98% or 99% identity, and includes subranges such as 96-99%, 96-98%, 96-97%.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value should be assumed.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures. To facilitate an understanding of the present disclosure, a number of terms and phrases are defined below.

The term “pharmaceutically acceptable” refers to approved or approvable by a regulatory agency of the Federal or a state government or listed in the U.S. Pharmacopeia or other generally recognized pharmacopeia for use in animals, including humans.

A “pharmaceutically acceptable carrier, excipient, or additive” refers to a carrier, excipient, or additive that can be administered to a subject, together with an agent, and which does not destroy the pharmacological activity thereof and is nontoxic when administered in doses sufficient to deliver a therapeutic amount of the agent.

As used herein, the terms “prevent,” “preventing,” “prevention,” and the like, refer to reducing the probability of developing a disease or condition in a subject, who does not have, but is at risk of or susceptible to developing a disease or condition.

The terms “treat,” “treated,” “treating,” “treatment,” and the like are meant to refer to reducing or ameliorating a disorder and/or symptoms associated therewith. “Treating” may refer to administration of the composition to a subject after the onset, or suspected onset, of a disease. “Treating” includes the concepts of “alleviating”, which refers to lessening the frequency of occurrence or recurrence, or the severity, of any symptoms or other ill effects related to a disease and/or the side effects associated with the disease. The term “treating” also encompasses the concept of “managing” which refers to reducing the severity of a particular disease or disorder in a patient or delaying its recurrence. The term “treating” further encompasses the concept of “prevent,” “preventing,” and “prevention,” as previously stated. It is appreciated that, although not precluded, treating a disorder or condition does not require that the disorder, condition, or symptoms associated therewith be completely eliminated.

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise.

### II. Non-Standard Amino Acid Containing Compositions

The use of non-standard amino acids in proteins offers the possibility of polypeptides having greatly expanded functionality that could be exploited for wide range of applications. For example, by incorporation of selenocysteine into polypeptides it may be possible to develop enzymes having enhanced levels of stability or activity and to produce highly active therapeutic polypeptides. However, these approaches have, to date, been hampered by the inability to produce organisms that stably retain translation pathways that predictable and reliably incorporate selenocysteine into encoded polypeptides. Studies detailed herein demonstrate a stable system for use of tRNA molecules that can incorporate selenocysteine and for production of polypeptides that incorporate selenocysteine positions. Importantly, this system can be easily moved from one organism to another without the need of re-engineering.

Over 100 NSAAs with diverse chemistries have been synthesized and co-translationally incorporated into proteins using evolved orthogonal aminoacyl-tRNA synthetase (airs)/tRNA pairs. Non-standard amino acids have been designed based on tyrosine or pyrrolysine. An aaRS/tRNA may be provided on a plasmid or into the genome of the genomically recoded organism. An orthogonal aaRS/tRNA pair will be used to bioorthogonally incorporate NSAAs into proteins. Vector-based over-expression systems may be used to outcompete natural codon function with its reassigned function. If one completely abolishes natural UAG translation function, far lower aaRS/tRNA function may be sufficient to achieve efficient NSAA incorporation. Genomically recoded organism (GRO)-based NSAA incorporation can use either vector- and/or genome-based aaRS/tRNA pairs. Genome-based aaRS/tRNA pairs have been used to reduce the mis-incorporation of standard amino acids in the absence of available NSAAs. Since the UAG codon function has been completely reassigned in the genomically recoded organism, NSAAs, such as selenocysteine, can be incorporated in the genomically recoded organism without any phenotypic consequences. NSAA incorporation in the genomically recoded organism may involve supplementing the growth media with the non-standard amino acid, such as selenocysteine, and an inducer for the aaRS. Alternatively, the aaRS may be expressed constitutively. Alternatively, as in the present disclosure, the endogenous seryl-tRNA synthetase may be used to serylate selenocysteine tRNA, which tRNA is acted upon by enzymes comprising SelA to produce tRNasec (selenocysteine charged tRNA). Media may be supplemented with a selenium source like sodium selenite to improve production of tRNasec. The desired protein can be overexpressed using any desired protein overexpression system (e.g., T7-RNAP, constitutive incorporation, or inducible expression based on IPTG/allolactose, anhydrotetracycline, arabinose, rhamnose, or other inducible systems). The protein cross-link (diselenide bond) may form spontaneously based on proximity-based geometric catalysis during protein folding, and the protein can be handled as any other over-expressed product.

The inventors have developed polypeptides and methods to produce polypeptides in genomically recoded organisms (GRO) that fold into biologics that, for example, are stabilized by diselenide bonds between selenocysteine amino acids. Whereas disulfide bonds between cysteine amino acids have a redox potential of about −220 mV, diselenide bonds have a redox potential of about −380 mV. Since the bacterial cytosol typically has a redox potential of about −280 to −300 mV, diselenides but not disulfides avoid reduction so that they form and persist in the cytosol. Since diselenides have the same geometric bond angles and torsions as disulfides, as well as very similar bond lengths, they can be substituted into polypeptides without disrupting the three-dimensional structure of the polypeptide. Further, since intended in vivo environments like blood contain reducing agents like glutathione, albumin, and thioredoxin, disulfides in polypeptides can be reduced, causing the polypeptide to unfold and, in the case of multiple disulfides, “scramble” the disulfides so that incorrect cysteines are bonded to each other. Both of these result in abrogation of the intended biological activity of the polypeptide. The lower redox potential of diselenides renders them resistant to reduction when exposed to blood serum or purified reducing components of blood serum, endowing them with a longer blood serum half-life than disulfide-bearing counterparts.

While peptides bearing diselenide-forming selenocysteines may be produced in vitro by solid phase peptide synthesis, the process does not scale tractably to the yields necessary for therapeutic applications, particularly for proteins. However, in vivo production of recombinant seleno-proteins is limited by strict sequence requirements on where selenocysteine may appear in proteins. In particular, a selenocysteine insertion sequence (SECIS) element must appear in the coding DNA sequence at the selenocysteine incorporation site in order to recruit endogenous selenocysteine translation machinery, comprising a specialized elongation factor (SelB). Instead, a recoded strain of E. coli can be used, which has an unassigned codon, such as an amber stop codon, together with an engineered selenocysteine tRNA with an anti-amber anticodon that permits targeted placement of selenocysteine into polypeptides by introduction of the amber stop codon into the corresponding DNA coding sequence. The modified tRNA interacts with the endogenous elongation factor EF-Tu. Other codons can be recoded, typically rare codons, as is known in the art. A codon on an mRNA and an anti-codon on a tRNA are typically triplets of complementary base sequences.

Recoded proteins may be synthesized in bacteria, such as E. coli cells, or in vitro, in translation or linked transcription-translation systems. Genes or mRNA encoding such recoded proteins are non-naturally occurring, and are variants of naturally occurring coding sequences. Although many of the proteins that we show in the associated sequence listing have all cysteine residues which participate in disulfide bonds replaced with selenocysteine residues, all cysteine residues need not be replaced to gain the benefits of the substitution. Even one diselenide bond may improve the stability of a protein. Any number of diselenide bonds (selenocysteine pairs) may be substituted for disulfide bonds in the proteins. If a protein has N disulfide bonds, the protein may have anywhere from N, N minus 1, N minus 2, N minus 3, N minus 4, . . . down to 1 such bond. It is also possible to form a bond between cysteine and selenocysteine residues called a selenylsulfide. This bond has a lower redox potential (˜−270 my) than a disulfide (−220 my) but not than bacterial cytoplasm (−280 my). The selenylsulfide bond may be used to increase resistance to reduction in certain redox environments. Selenylsulfides may be used in place of diselenides using methods described here by substituting selenocysteine for a single disulfide bonded cysteine, or by substituting cysteine for a single diselenide bonded selenocysteine.

Sequences of disulfide-stabilized biologics with substituted selenocysteines can be produced in the cytosol of E. coli using our method at the mg/L scale in standard laboratory shaker flasks, and scaled to g/L production in microbial fermenters.

Enzymes with different combinations of diselenide bonds and disulfides include, but are not limited to, nucleases (such as DNases and RNases), polymerases, ligases, reverse transcriptases, proteases, restriction endonucleases, and carbon fixing enzymes (e.g., carbon capturing enzymes).

Any cysteine in an enzyme disclosed herein may be maintained as a selenocysteine so long as the presence of the selenocysteine does not interfere with the expression, folding, or intended function of the polypeptide. Methods are provided herein for producing and verifying the presence of selenocysteines participating in the intended diselenide bonds for various enzymes, including, but not limited to, nucleases (such as DNases and RNases), polymerases, ligases, reverse transcriptases, proteases, restriction endonucleases, and carbon fixing enzymes (e.g., carbon capturing enzymes).

Stabilized enzymes may be made and used according to the invention with diselenide bonds between two selenocysteine residues. This technique and modification can be useful for producing enzymes that maintain activity even in harsh conditions such as reducing environments. Provided herein are stabilized enzymes containing non-standard amino acids that have enzymatic activity in harsh conditions, such as reducing buffers or lysis buffers, that is higher than a corresponding enzyme without the non-standard amino acids under the same conditions. The stabilized enzymes can comprise a stabilized DNase I polypeptide. Also provided herein are polynucleotides encoding these stabilized enzymes, cells for expressing and/or producing these stabilized enzymes, and methods of use of these stabilized enzymes.

Enzymes with different combinations of diselenide bonds and disulfides include, but are not limited to, nucleases (such as DNases and RNases), polymerases, ligases, reverse transcriptases, proteases, restriction endonucleases, and carbon fixing enzymes (e.g., carbon capturing enzymes).

In some embodiments, a nuclease may be made and used according to the invention with diselenide bonds between two selenocysteine residues. Exemplary nucleases include, but are not limited to, DNases (e.g., bovine DNase I), RNases and the like. For example, DNase I has two disulfide bonds. For example, RNase A has 4 disulfide bonds. In some embodiments, a RNase A enzyme comprises 2, 4, 6, or 8 selenocysteine residues. In some embodiments, a RNase A enzyme comprises at least 1, 2, 3, or 4 diselenide bonds. For example, RNase 3 has 4 disulfide bonds. In some embodiments, a RNase 3 enzyme comprises at least 2, 4, 6, or 8 selenocysteine residues. In some embodiments, a RNase 3 enzyme comprises at least 1, 2, 3, or 4 diselenide bonds. For example, benzonase (e.g., Serratia marcescens nuclease) comprises at least two essential disulfide bonds and is a 30 kDa homodimer. In some embodiments, a benzonase comprises at least 2 or 4 selenocysteine residues. In some embodiments, a benzonase comprises at least 1 or 2 diselenide bonds.

In some embodiments, a nuclease can comprise one or more non-standard amino acids. In some embodiments, a nuclease can comprise one or more selenocysteine residues. In some embodiments, a nuclease can comprise a diselenide bond between two selenocysteine residues. The diselenide bonds may be intramolecular or intermolecular. In some embodiments, a nuclease can comprise one or more diselenide bonds. In some embodiments, a nuclease comprising one or more non-standard amino acids has enzymatic activity in harsh conditions, such as reducing buffers or lysis buffers, that is higher than a corresponding nuclease without the non-standard amino acids under the same conditions.

For example, a nuclease provided herein comprising one or more non-standard amino acids, such as one or more selenocysteine residues, can cleave a bond of a polynucleotide substrate with an activity that is at least 1.1, 1.2, 1.3, 1.4, 1.5, 1.6, 1.7, 1.8, 1.9, 2, 2.1, 2.2, 2.3, 2.4, 2.5, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20, 25, 30, 35, 40, 45, 50, 55, 60, 65, 70, 75, 80, 85, 90, 95, 100, 200, 300, 400, 500, 600, 700, 800, 900, or 1000 times higher than a corresponding nuclease without the one or more non-standard amino acids.

For example, a nuclease provided herein comprising one or more non-standard amino acids, such as one or more selenocysteine residues, can cleave a bond of a polynucleotide substrate in a buffer with an activity that is at least 1.1, 1.2, 1.3, 1.4, 1.5, 1.6, 1.7, 1.8, 1.9, 2, 2.1, 2.2, 2.3, 2.4, 2.5, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20, 25, 30, 35, 40, 45, 50, 55, 60, 65, 70, 75, 80, 85, 90, 95, 100, 200, 300, 400, 500, 600, 700, 800, 900, or 1000 times higher than a corresponding nuclease without the one or more non-standard amino acids in the same buffer.

For example, a nuclease provided herein comprising one or more non-standard amino acids, such as one or more selenocysteine residues, can cleave a bond of a polynucleotide substrate in a buffer comprising a detergent, a reducing reagent, and/or a reducing enzyme (e.g., a reductase) with an activity that is at least 1.1, 1.2, 1.3, 1.4, 1.5, 1.6, 1.7, 1.8, 1.9, 2, 2.1, 2.2, 2.3, 2.4, 2.5, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20, 25, 30, 35, 40, 45, 50, 55, 60, 65, 70, 75, 80, 85, 90, 95, 100, 200, 300, 400, 500, 600, 700, 800, 900, or 1000 times higher than a corresponding nuclease without the one or more non-standard amino acids in the same buffer comprising the detergent, the reducing reagent, and/or the reducing enzyme (e.g., a reductase).

For example, a nuclease provided herein comprising one or more non-standard amino acids, such as one or more selenocysteine residues, can cleave a bond of a polynucleotide substrate in a buffer with a redox potential of less than about −150 mV, with an activity that is at least 1.1, 1.2, 1.3, 1.4, 1.5, 1.6, 1.7, 1.8, 1.9, 2, 2.1, 2.2, 2.3, 2.4, 2.5, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20, 25, 30, 35, 40, 45, 50, 55, 60, 65, 70, 75, 80, 85, 90, 95, 100, 200, 300, 400, 500, 600, 700, 800, 900, or 1000 times higher than a corresponding nuclease without the one or more non-standard amino acids in a buffer with the same redox potential. For example, a nuclease provided herein comprising one or more non-standard amino acids, such as one or more selenocysteine residues, can cleave a bond of a polynucleotide substrate in a buffer with a redox potential of less than about −160 mV, less than about −170 mV, less than about −180 mV, less than about −190 mV, less than about −200 mV, less than about −210 mV, less than about −220 mV, less than about −230 mV, less than about −240 mV, or less than about −250 mV, less than about −260 mV, less than about −270 mV, less than about −280 mV, less than about −290 mV, less than about −300 mV, less than about −310 mV, less than about −320 mV, less than about −330 mV, less than about −340 mV, or less than about −350 mV, less than about −360 mV, less than about −370 mV, less than about −380 mV, less than about −390 mV, less than about −400 mV, less than about −410 mV, less than about −420 mV, less than about −430 mV, less than about −440 mV, or less than about −450 mV, less than about −460 mV, less than about −470 mV, less than about −480 mV, less than about −490 mV, less than about −500 mV, less than about −510 mV, less than about −520 mV, less than about −530 mV, less than about −540 mV, or less than about −550 mV, less than about −560 mV, less than about −570 mV, less than about −580 mV, less than about −590 mV, or less than about −600 mV, with an activity that is at least 1.1, 1.2, 1.3, 1.4, 1.5, 1.6, 1.7, 1.8, 1.9, 2, 2.1, 2.2, 2.3, 2.4, 2.5, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20, 25, 30, 35, 40, 45, 50, 55, 60, 65, 70, 75, 80, 85, 90, 95, 100, 200, 300, 400, 500, 600, 700, 800, 900, or 1000 times higher than a corresponding nuclease without the one or more non-standard amino acids in a buffer with the same redox potential.

In some embodiments, a polymerase may be made and used according to the invention with diselenide bonds between two selenocysteine residues. In some embodiments, a polymerase can comprise one or more non-standard amino acids. In some embodiments, a polymerase can comprise one or more selenocysteine residues. In some embodiments, a polymerase can comprise a diselenide bond between two selenocysteine residues. The diselenide bonds may be intramolecular or intermolecular. In some embodiments, a polymerase can comprise one or more diselenide bonds. In some embodiments, a polymerase comprising one or more non-standard amino acids has enzymatic activity in harsh conditions, such as reducing buffers or lysis buffers, that is higher than a corresponding polymerase without the non-standard amino acids under the same conditions.

For example, a polymerase provided herein comprising one or more non-standard amino acids, such as one or more selenocysteine residues, can catalyze a polymerase reaction with an activity that is at least 1.1, 1.1, 1.2, 1.3, 1.4, 1.5, 1.6, 1.7, 1.8, 1.9, 2, 2.1, 2.2, 2.3, 2.4, 2.5, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20, 25, 30, 35, 40, 45, 50, 55, 60, 65, 70, 75, 80, 85, 90, 95, 100, 200, 300, 400, 500, 600, 700, 800, 900, or 1000 times higher than a corresponding polymerase without the one or more non-standard amino acids.

For example, a polymerase provided herein comprising one or more non-standard amino acids, such as one or more selenocysteine residues, can catalyze a polymerase reaction in a buffer with an activity that is at least 1.1, 1.2, 1.3, 1.4, 1.5, 1.6, 1.7, 1.8, 1.9, 2, 2.1, 2.2, 2.3, 2.4, 2.5, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20, 25, 30, 35, 40, 45, 50, 55, 60, 65, 70, 75, 80, 85, 90, 95, 100, 200, 300, 400, 500, 600, 700, 800, 900, or 1000 times higher than a corresponding polymerase without the one or more non-standard amino acids in the same buffer.

For example, a polymerase provided herein comprising one or more non-standard amino acids, such as one or more selenocysteine residues, can catalyze a polymerase reaction in a buffer comprising a detergent, a reducing reagent, and/or a reducing enzyme (e.g., a reductase) with an activity that is at least 1.1, 1.1, 1.2, 1.3, 1.4, 1.5, 1.6, 1.7, 1.8, 1.9, 2, 2.1, 2.2, 2.3, 2.4, 2.5, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20, 25, 30, 35, 40, 45, 50, 55, 60, 65, 70, 75, 80, 85, 90, 95, 100, 200, 300, 400, 500, 600, 700, 800, 900, or 1000 times higher than a corresponding polymerase without the one or more non-standard amino acids in the same buffer comprising the detergent, the reducing reagent, and/or the reducing enzyme (e.g., a reductase).

For example, a polymerase provided herein comprising one or more non-standard amino acids, such as one or more selenocysteine residues, can catalyze a polymerase reaction in a buffer with a redox potential of less than about −150 mV, with an activity that is at least 1.1, 1.2, 1.3, 1.4, 1.5, 1.6, 1.7, 1.8, 1.9, 2, 2.1, 2.2, 2.3, 2.4, 2.5, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20, 25, 30, 35, 40, 45, 50, 55, 60, 65, 70, 75, 80, 85, 90, 95, 100, 200, 300, 400, 500, 600, 700, 800, 900, or 1000 times higher than a corresponding polymerase without the one or more non-standard amino acids in a buffer with the same redox potential. For example, a polymerase provided herein comprising one or more non-standard amino acids, such as one or more selenocysteine residues, can catalyze a polymerase reaction in a buffer with a redox potential of less than about −160 mV, less than about −170 mV, less than about −180 mV, less than about −190 mV, less than about −200 mV, less than about −210 mV, less than about −220 mV, less than about −230 mV, less than about −240 mV, or less than about −250 mV, less than about −260 mV, less than about −270 mV, less than about −280 mV, less than about −290 mV, less than about −300 mV, less than about −310 mV, less than about −320 mV, less than about −330 mV, less than about −340 mV, or less than about −350 mV, less than about −360 mV, less than about −370 mV, less than about −380 mV, less than about −390 mV, less than about −400 mV, less than about −410 mV, less than about −420 mV, less than about −430 mV, less than about −440 mV, or less than about −450 mV, less than about −460 mV, less than about −470 mV, less than about −480 mV, less than about −490 mV, less than about −500 mV, less than about −510 mV, less than about −520 mV, less than about −530 mV, less than about −540 mV, or less than about −550 mV, less than about −560 mV, less than about −570 mV, less than about −580 mV, less than about −590 mV, or less than about −600 mV, with an activity that is at least 1.1, 1.2, 1.3, 1.4, 1.5, 1.6, 1.7, 1.8, 1.9, 2, 2.1, 2.2, 2.3, 2.4, 2.5, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20, 25, 30, 35, 40, 45, 50, 55, 60, 65, 70, 75, 80, 85, 90, 95, 100, 200, 300, 400, 500, 600, 700, 800, 900, or 1000 times higher than a corresponding polymerase without the one or more non-standard amino acids in a buffer with the same redox potential.

In some embodiments, a ligase may be made and used according to the invention with diselenide bonds between two selenocysteine residues. In some embodiments, a ligase can comprise one or more non-standard amino acids. In some embodiments, a ligase can comprise one or more selenocysteine residues. In some embodiments, a ligase can comprise a diselenide bond between two selenocysteine residues. The diselenide bonds may be intramolecular or intermolecular. In some embodiments, a ligase can comprise one or more diselenide bonds. In some embodiments, a ligase comprising one or more non-standard amino acids has enzymatic activity in harsh conditions, such as reducing buffers or lysis buffers, that is higher than a corresponding ligase without the non-standard amino acids under the same conditions.

For example, a ligase provided herein comprising one or more non-standard amino acids, such as one or more selenocysteine residues, can ligate two or more nucleic acids together with an activity that is at least 1.1, 1.2, 1.3, 1.4, 1.5, 1.6, 1.7, 1.8, 1.9, 2, 2.1, 2.2, 2.3, 2.4, 2.5, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20, 25, 30, 35, 40, 45, 50, 55, 60, 65, 70, 75, 80, 85, 90, 95, 100, 200, 300, 400, 500, 600, 700, 800, 900, or 1000 times higher than a corresponding ligase without the one or more non-standard amino acids.

For example, a ligase provided herein comprising one or more non-standard amino acids, such as one or more selenocysteine residues, can ligate two or more nucleic acids together in a buffer with an activity that is at least 1.1, 1.2, 1.3, 1.4, 1.5, 1.6, 1.7, 1.8, 1.9, 2, 2.1, 2.2, 2.3, 2.4, 2.5, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20, 25, 30, 35, 40, 45, 50, 55, 60, 65, 70, 75, 80, 85, 90, 95, 100, 200, 300, 400, 500, 600, 700, 800, 900, or 1000 times higher than a corresponding ligase without the one or more non-standard amino acids in the same buffer.

For example, a ligase provided herein comprising one or more non-standard amino acids, such as one or more selenocysteine residues, can ligate two or more nucleic acids together in a buffer comprising a detergent, a reducing reagent, and/or a reducing enzyme (e.g., a reductase) with an activity that is at least 1.1, 1.2, 1.3, 1.4, 1.5, 1.6, 1.7, 1.8, 1.9, 2, 2.1, 2.2, 2.3, 2.4, 2.5, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20, 25, 30, 35, 40, 45, 50, 55, 60, 65, 70, 75, 80, 85, 90, 95, 100, 200, 300, 400, 500, 600, 700, 800, 900, or 1000 times higher than a corresponding ligase without the one or more non-standard amino acids in the same buffer comprising the detergent, the reducing reagent, and/or the reducing enzyme (e.g., a reductase).

For example, a ligase provided herein comprising one or more non-standard amino acids, such as one or more selenocysteine residues, can ligate two or more nucleic acids together in a buffer with a redox potential of less than about −150 mV, with an activity that is at least 1.1, 1.2, 1.3, 1.4, 1.5, 1.6, 1.7, 1.8, 1.9, 2, 2.1, 2.2, 2.3, 2.4, 2.5, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20, 25, 30, 35, 40, 45, 50, 55, 60, 65, 70, 75, 80, 85, 90, 95, 100, 200, 300, 400, 500, 600, 700, 800, 900, or 1000 times higher than a corresponding ligase without the one or more non-standard amino acids in a buffer with the same redox potential. For example, a ligase provided herein comprising one or more non-standard amino acids, such as one or more selenocysteine residues, can ligate two or more nucleic acids together in a buffer with a redox potential of less than about −160 mV, less than about −170 mV, less than about −180 mV, less than about −190 mV, less than about −200 mV, less than about −210 mV, less than about −220 mV, less than about −230 mV, less than about −240 mV, or less than about −250 mV, less than about −260 mV, less than about −270 mV, less than about −280 mV, less than about −290 mV, less than about −300 mV, less than about −310 mV, less than about −320 mV, less than about −330 mV, less than about −340 mV, or less than about −350 mV, less than about −360 mV, less than about −370 mV, less than about −380 mV, less than about −390 mV, less than about −400 mV, less than about −410 mV, less than about −420 mV, less than about −430 mV, less than about −440 mV, or less than about −450 mV, less than about −460 mV, less than about −470 mV, less than about −480 mV, less than about −490 mV, less than about −500 mV, less than about −510 mV, less than about −520 mV, less than about −530 mV, less than about −540 mV, or less than about −550 mV, less than about −560 mV, less than about −570 mV, less than about −580 mV, less than about −590 mV, or less than about −600 mV, with an activity that is at least 1.1, 1.2, 1.3, 1.4, 1.5, 1.6, 1.7, 1.8, 1.9, 2, 2.1, 2.2, 2.3, 2.4, 2.5, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20, 25, 30, 35, 40, 45, 50, 55, 60, 65, 70, 75, 80, 85, 90, 95, 100, 200, 300, 400, 500, 600, 700, 800, 900, or 1000 times higher than a corresponding ligase without the one or more non-standard amino acids in a buffer with the same redox potential.

In some embodiments, a restriction endonuclease may be made and used according to the invention with diselenide bonds between two selenocysteine residues. In some embodiments, a restriction endonuclease can comprise one or more non-standard amino acids. In some embodiments, a restriction endonuclease can comprise one or more selenocysteine residues. In some embodiments, a restriction endonuclease can comprise a diselenide bond between two selenocysteine residues. The diselenide bonds may be intramolecular or intermolecular. In some embodiments, a restriction endonuclease can comprise one or more diselenide bonds. In some embodiments, a restriction endonuclease comprising one or more non-standard amino acids has enzymatic activity in harsh conditions, such as reducing buffers or lysis buffers, that is higher than a corresponding restriction endonuclease without the non-standard amino acids under the same conditions.

For example, a restriction endonuclease provided herein comprising one or more non-standard amino acids, such as one or more selenocysteine residues, can cleave one or more bonds of a polynucleotide substrate with an activity that is at least 1.1, 1.2, 1.3, 1.4, 1.5, 1.6, 1.7, 1.8, 1.9, 2, 2.1, 2.2, 2.3, 2.4, 2.5, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20, 25, 30, 35, 40, 45, 50, 55, 60, 65, 70, 75, 80, 85, 90, 95, 100, 200, 300, 400, 500, 600, 700, 800, 900, or 1000 times higher than a corresponding restriction endonuclease without the one or more non-standard amino acids.

For example, a restriction endonuclease provided herein comprising one or more non-standard amino acids, such as one or more selenocysteine residues, can cleave one or more bonds of a polynucleotide substrate in a buffer with an activity that is at least 1.1, 1.2, 1.3, 1.4, 1.5, 1.6, 1.7, 1.8, 1.9, 2, 2.1, 2.2, 2.3, 2.4, 2.5, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20, 25, 30, 35, 40, 45, 50, 55, 60, 65, 70, 75, 80, 85, 90, 95, 100, 200, 300, 400, 500, 600, 700, 800, 900, or 1000 times higher than a corresponding restriction endonuclease without the one or more non-standard amino acids in the same buffer.

For example, a restriction endonuclease provided herein comprising one or more non-standard amino acids, such as one or more selenocysteine residues, can cleave one or more bonds of a polynucleotide substrate in a buffer comprising a detergent, a reducing reagent, and/or a reducing enzyme (e.g., a reductase) with an activity that is at least 1.1, 1.2, 1.3, 1.4, 1.5, 1.6, 1.7, 1.8, 1.9, 2, 2.1, 2.2, 2.3, 2.4, 2.5, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20, 25, 30, 35, 40, 45, 50, 55, 60, 65, 70, 75, 80, 85, 90, 95, 100, 200, 300, 400, 500, 600, 700, 800, 900, or 1000 times higher than a corresponding restriction endonuclease without the one or more non-standard amino acids in the same buffer comprising the detergent, the reducing reagent, and/or the reducing enzyme (e.g., a reductase).

For example, a restriction endonuclease provided herein comprising one or more non-standard amino acids, such as one or more selenocysteine residues, can cleave one or more bonds of a polynucleotide substrate in a buffer with a redox potential of less than about −150 mV, with an activity that is at least 1.1, 1.2, 1.3, 1.4, 1.5, 1.6, 1.7, 1.8, 1.9, 2, 2.1, 2.2, 2.3, 2.4, 2.5, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20, 25, 30, 35, 40, 45, 50, 55, 60, 65, 70, 75, 80, 85, 90, 95, 100, 200, 300, 400, 500, 600, 700, 800, 900, or 1000 times higher than a corresponding restriction endonuclease without the one or more non-standard amino acids in a buffer with the same redox potential. For example, a restriction endonuclease provided herein comprising one or more non-standard amino acids, such as one or more selenocysteine residues, can cleave one or more bonds of a polynucleotide substrate in a buffer with a redox potential of less than about −160 mV, less than about −170 mV, less than about −180 mV, less than about −190 mV, less than about −200 mV, less than about −210 mV, less than about −220 mV, less than about −230 mV, less than about −240 mV, or less than about −250 mV, less than about −260 mV, less than about −270 mV, less than about −280 mV, less than about −290 mV, less than about −300 mV, less than about −310 mV, less than about −320 mV, less than about −330 mV, less than about −340 mV, or less than about −350 mV, less than about −360 mV, less than about −370 mV, less than about −380 mV, less than about −390 mV, less than about −400 mV, less than about −410 mV, less than about −420 mV, less than about −430 mV, less than about −440 mV, or less than about −450 mV, less than about −460 mV, less than about −470 mV, less than about −480 mV, less than about −490 mV, less than about −500 mV, less than about −510 mV, less than about −520 mV, less than about −530 mV, less than about −540 mV, or less than about −550 mV, less than about −560 mV, less than about −570 mV, less than about −580 mV, less than about −590 mV, or less than about −600 mV, with an activity that is at least 1.1, 1.2, 1.3, 1.4, 1.5, 1.6, 1.7, 1.8, 1.9, 2, 2.1, 2.2, 2.3, 2.4, 2.5, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20, 25, 30, 35, 40, 45, 50, 55, 60, 65, 70, 75, 80, 85, 90, 95, 100, 200, 300, 400, 500, 600, 700, 800, 900, or 1000 times higher than a corresponding restriction endonuclease without the one or more non-standard amino acids in a buffer with the same redox potential.

In some embodiments, a reverse transcriptase may be made and used according to the invention with diselenide bonds between two selenocysteine residues. In some embodiments, a reverse transcriptase can comprise one or more non-standard amino acids. In some embodiments, a reverse transcriptase can comprise one or more selenocysteine residues. In some embodiments, a reverse transcriptase can comprise a diselenide bond between two selenocysteine residues. The diselenide bonds may be intramolecular or intermolecular. In some embodiments, a reverse transcriptase can comprise one or more diselenide bonds. In some embodiments, a reverse transcriptase comprising one or more non-standard amino acids has enzymatic activity in harsh conditions, such as reducing buffers or lysis buffers, that is higher than a corresponding reverse transcriptase without the non-standard amino acids under the same conditions.

For example, a reverse transcriptase provided herein comprising one or more non-standard amino acids, such as one or more selenocysteine residues, can synthesize a cDNA from an RNA with an activity that is at least 1.1, 1.2, 1.3, 1.4, 1.5, 1.6, 1.7, 1.8, 1.9, 2, 2.1, 2.2, 2.3, 2.4, 2.5, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20, 25, 30, 35, 40, 45, 50, 55, 60, 65, 70, 75, 80, 85, 90, 95, 100, 200, 300, 400, 500, 600, 700, 800, 900, or 1000 times higher than a corresponding reverse transcriptase without the one or more non-standard amino acids.

For example, a reverse transcriptase provided herein comprising one or more non-standard amino acids, such as one or more selenocysteine residues, can synthesize a cDNA from an RNA in a buffer with an activity that is at least 1.1, 1.2, 1.3, 1.4, 1.5, 1.6, 1.7, 1.8, 1.9, 2, 2.1, 2.2, 2.3, 2.4, 2.5, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20, 25, 30, 35, 40, 45, 50, 55, 60, 65, 70, 75, 80, 85, 90, 95, 100, 200, 300, 400, 500, 600, 700, 800, 900, or 1000 times higher than a corresponding reverse transcriptase without the one or more non-standard amino acids in the same buffer.

For example, a reverse transcriptase provided herein comprising one or more non-standard amino acids, such as one or more selenocysteine residues, can synthesize a cDNA from an RNA in a buffer comprising a detergent, a reducing reagent, and/or a reducing enzyme (e.g., a reductase) with an activity that is at least 1.1, 1.2, 1.3, 1.4, 1.5, 1.6, 1.7, 1.8, 1.9, 2, 2.1, 2.2, 2.3, 2.4, 2.5, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20, 25, 30, 35, 40, 45, 50, 55, 60, 65, 70, 75, 80, 85, 90, 95, 100, 200, 300, 400, 500, 600, 700, 800, 900, or 1000 times higher than a corresponding reverse transcriptase without the one or more non-standard amino acids in the same buffer comprising the detergent, the reducing reagent, and/or the reducing enzyme (e.g., a reductase).

For example, a reverse transcriptase provided herein comprising one or more non-standard amino acids, such as one or more selenocysteine residues, can synthesize a cDNA from an RNA in a buffer with a redox potential of less than about −150 mV, with an activity that is at least 1.1, 1.2, 1.3, 1.4, 1.5, 1.6, 1.7, 1.8, 1.9, 2, 2.1, 2.2, 2.3, 2.4, 2.5, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20, 25, 30, 35, 40, 45, 50, 55, 60, 65, 70, 75, 80, 85, 90, 95, 100, 200, 300, 400, 500, 600, 700, 800, 900, or 1000 times higher than a corresponding reverse transcriptase without the one or more non-standard amino acids in a buffer with the same redox potential. For example, a reverse transcriptase provided herein comprising one or more non-standard amino acids, such as one or more selenocysteine residues, can synthesize a cDNA from an RNA in a buffer with a redox potential of less than about −160 mV, less than about −170 mV, less than about −180 mV, less than about −190 mV, less than about −200 mV, less than about −210 mV, less than about −220 mV, less than about −230 mV, less than about −240 mV, or less than about −250 mV, less than about −260 mV, less than about −270 mV, less than about −280 mV, less than about −290 mV, less than about −300 mV, less than about −310 mV, less than about −320 mV, less than about −330 mV, less than about −340 mV, or less than about −350 mV, less than about −360 mV, less than about −370 mV, less than about −380 mV, less than about −390 mV, less than about −400 mV, less than about −410 mV, less than about −420 mV, less than about −430 mV, less than about −440 mV, or less than about −450 mV, less than about −460 mV, less than about −470 mV, less than about −480 mV, less than about −490 mV, less than about −500 mV, less than about −510 mV, less than about −520 mV, less than about −530 mV, less than about −540 mV, or less than about −550 mV, less than about −560 mV, less than about −570 mV, less than about −580 mV, less than about −590 mV, or less than about −600 mV, with an activity that is at least 1.1, 1.2, 1.3, 1.4, 1.5, 1.6, 1.7, 1.8, 1.9, 2, 2.1, 2.2, 2.3, 2.4, 2.5, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20, 25, 30, 35, 40, 45, 50, 55, 60, 65, 70, 75, 80, 85, 90, 95, 100, 200, 300, 400, 500, 600, 700, 800, 900, or 1000 times higher than a corresponding reverse transcriptase without the one or more non-standard amino acids in a buffer with the same redox potential.

In some embodiments, a protease may be made and used according to the invention with diselenide bonds between two selenocysteine residues. In some embodiments, a protease can comprise one or more non-standard amino acids. In some embodiments, a protease can comprise one or more selenocysteine residues. In some embodiments, a protease can comprise a diselenide bond between two selenocysteine residues. The diselenide bonds may be intramolecular or intermolecular. In some embodiments, a protease can comprise one or more diselenide bonds. In some embodiments, a protease comprising one or more non-standard amino acids has enzymatic activity in harsh conditions, such as reducing buffers or lysis buffers, that is higher than a corresponding protease without the non-standard amino acids under the same conditions.

For example, a protease provided herein comprising one or more non-standard amino acids, such as one or more selenocysteine residues, can cleave a bond of a polypeptide substrate with an activity that is at least 1.1, 1.2, 1.3, 1.4, 1.5, 1.6, 1.7, 1.8, 1.9, 2, 2.1, 2.2, 2.3, 2.4, 2.5, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20, 25, 30, 35, 40, 45, 50, 55, 60, 65, 70, 75, 80, 85, 90, 95, 100, 200, 300, 400, 500, 600, 700, 800, 900, or 1000 times higher than a corresponding protease without the one or more non-standard amino acids.

For example, a protease provided herein comprising one or more non-standard amino acids, such as one or more selenocysteine residues, can cleave a bond of a polypeptide substrate in a buffer with an activity that is at least 1.1, 1.2, 1.3, 1.4, 1.5, 1.6, 1.7, 1.8, 1.9, 2, 2.1, 2.2, 2.3, 2.4, 2.5, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20, 25, 30, 35, 40, 45, 50, 55, 60, 65, 70, 75, 80, 85, 90, 95, 100, 200, 300, 400, 500, 600, 700, 800, 900, or 1000 times higher than a corresponding protease without the one or more non-standard amino acids in the same buffer.

For example, a protease provided herein comprising one or more non-standard amino acids, such as one or more selenocysteine residues, can cleave a bond of a polypeptide substrate in a buffer comprising a detergent, a reducing reagent, and/or a reducing enzyme (e.g., a reductase) with an activity that is at least 1.1, 1.2, 1.3, 1.4, 1.5, 1.6, 1.7, 1.8, 1.9, 2, 2.1, 2.2, 2.3, 2.4, 2.5, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20, 25, 30, 35, 40, 45, 50, 55, 60, 65, 70, 75, 80, 85, 90, 95, 100, 200, 300, 400, 500, 600, 700, 800, 900, or 1000 times higher than a corresponding protease without the one or more non-standard amino acids in the same buffer comprising the detergent, the reducing reagent, and/or the reducing enzyme (e.g., a reductase).

For example, a protease provided herein comprising one or more non-standard amino acids, such as one or more selenocysteine residues, can cleave a bond of a polypeptide substrate in a buffer with a redox potential of less than about −150 mV, with an activity that is at least 1.1, 1.2, 1.3, 1.4, 1.5, 1.6, 1.7, 1.8, 1.9, 2, 2.1, 2.2, 2.3, 2.4, 2.5, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20, 25, 30, 35, 40, 45, 50, 55, 60, 65, 70, 75, 80, 85, 90, 95, 100, 200, 300, 400, 500, 600, 700, 800, 900, or 1000 times higher than a corresponding protease without the one or more non-standard amino acids in a buffer with the same redox potential. For example, a protease provided herein comprising one or more non-standard amino acids, such as one or more selenocysteine residues, can cleave a bond of a polypeptide substrate in a buffer with a redox potential of less than about −160 mV, less than about −170 mV, less than about −180 mV, less than about −190 mV, less than about −200 mV, less than about −210 mV, less than about −220 mV, less than about −230 mV, less than about −240 mV, or less than about −250 mV, less than about −260 mV, less than about −270 mV, less than about −280 mV, less than about −290 mV, less than about −300 mV, less than about −310 mV, less than about −320 mV, less than about −330 mV, less than about −340 mV, or less than about −350 mV, less than about −360 mV, less than about −370 mV, less than about −380 mV, less than about −390 mV, less than about −400 mV, less than about −410 mV, less than about −420 mV, less than about −430 mV, less than about −440 mV, or less than about −450 mV, less than about −460 mV, less than about −470 mV, less than about −480 mV, less than about −490 mV, less than about −500 mV, less than about −510 mV, less than about −520 mV, less than about −530 mV, less than about −540 mV, or less than about −550 mV, less than about −560 mV, less than about −570 mV, less than about −580 mV, less than about −590 mV, or less than about −600 mV, with an activity that is at least 1.1, 1.2, 1.3, 1.4, 1.5, 1.6, 1.7, 1.8, 1.9, 2, 2.1, 2.2, 2.3, 2.4, 2.5, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20, 25, 30, 35, 40, 45, 50, 55, 60, 65, 70, 75, 80, 85, 90, 95, 100, 200, 300, 400, 500, 600, 700, 800, 900, or 1000 times higher than a corresponding protease without the one or more non-standard amino acids in a buffer with the same redox potential.

In some embodiments, an enzyme containing one or more catalytic cysteine residues (i.e. a cysteine involved in a catalysis reaction, e.g., an active site cysteine) may be made and used according to the invention with one or more selenocysteine residue substitutions for these one or more catalytic cysteine residues. The one or more selenocysteine subtitutions can increase or alter the enzyme activity in the reaction environment.

In some embodiments, a carbon capturing enzyme (e.g., a carbon fixing enzyme) may be made and used according to the invention with diselenide bonds between two selenocysteine residues. In some embodiments, a carbon capturing enzyme (e.g., a carbon fixing enzyme) can comprise one or more non-standard amino acids. In some embodiments, a carbon capturing enzyme (e.g., a carbon fixing enzyme) can comprise one or more selenocysteine residues. In some embodiments, a carbon capturing enzyme (e.g., a carbon fixing enzyme) can comprise a diselenide bond between two selenocysteine residues. The diselenide bonds may be intramolecular or intermolecular. In some embodiments, a carbon capturing enzyme (e.g., a carbon fixing enzyme) can comprise one or more diselenide bonds. In some embodiments, a carbon capturing enzyme (e.g., a carbon fixing enzyme) comprising one or more non-standard amino acids has enzymatic activity in harsh conditions, such as reducing buffers or lysis buffers, that is higher than a corresponding carbon capturing enzyme (e.g., a carbon fixing enzyme) without the non-standard amino acids under the same conditions. For example, in some embodiments, a carbon capturing enzyme (e.g., a carbon fixing enzyme), such as an anhydrase enzyme (e.g., β-carbonic anhydrase) can comprise one or more catalytic selenocysteine substitutions.

For example, a carbon capturing enzyme (e.g., a carbon fixing enzyme) provided herein comprising one or more non-standard amino acids, such as one or more selenocysteine residues, can capture or fix carbon with an activity that is at least 1.1, 1.2, 1.3, 1.4, 1.5, 1.6, 1.7, 1.8, 1.9, 2, 2.1, 2.2, 2.3, 2.4, 2.5, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20, 25, 30, 35, 40, 45, 50, 55, 60, 65, 70, 75, 80, 85, 90, 95, 100, 200, 300, 400, 500, 600, 700, 800, 900, or 1000 times higher than a corresponding carbon capturing enzyme (e.g., a carbon fixing enzyme) without the one or more non-standard amino acids. For example, an enzyme, such as a carbon capturing enzyme (e.g., a carbon fixing enzyme) provided herein comprising one or more non-standard active site amino acids, such as one or more active site selenocysteine residues can capture or fix carbon with an activity that is at least 1.1, 1.2, 1.3, 1.4, 1.5, 1.6, 1.7, 1.8, 1.9, 2, 2.1, 2.2, 2.3, 2.4, 2.5, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20, 25, 30, 35, 40, 45, 50, 55, 60, 65, 70, 75, 80, 85, 90, 95, 100, 200, 300, 400, 500, 600, 700, 800, 900, or 1000 times higher than a corresponding enzyme, such as a carbon capturing enzyme (e.g., a carbon fixing enzyme) without the one or more non-standard active site amino acids.

For example, a carbon capturing enzyme (e.g., a carbon fixing enzyme) provided herein comprising one or more non-standard amino acids, such as one or more selenocysteine residues, can capture or fix carbon in a buffer or environment with an activity that is at least 1.1, 1.2, 1.3, 1.4, 1.5, 1.6, 1.7, 1.8, 1.9, 2, 2.1, 2.2, 2.3, 2.4, 2.5, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20, 25, 30, 35, 40, 45, 50, 55, 60, 65, 70, 75, 80, 85, 90, 95, 100, 200, 300, 400, 500, 600, 700, 800, 900, or 1000 times higher than a corresponding carbon capturing enzyme (e.g., a carbon fixing enzyme) without the one or more non-standard amino acids in the same buffer or environment.

For example, a carbon capturing enzyme (e.g., a carbon fixing enzyme) provided herein comprising one or more non-standard amino acids, such as one or more selenocysteine residues, can capture or fix carbon in a buffer comprising a detergent, a reducing reagent, and/or a reducing enzyme (e.g., a reductase) or a reducing environment with an activity that is at least 1.1, 1.2, 1.3, 1.4, 1.5, 1.6, 1.7, 1.8, 1.9, 2, 2.1, 2.2, 2.3, 2.4, 2.5, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20, 25, 30, 35, 40, 45, 50, 55, 60, 65, 70, 75, 80, 85, 90, 95, 100, 200, 300, 400, 500, 600, 700, 800, 900, or 1000 times higher than a corresponding carbon capturing enzyme (e.g., a carbon fixing enzyme) without the one or more non-standard amino acids in the same buffer comprising the detergent, the reducing reagent, and/or the reducing enzyme (e.g., a reductase) or in the same reducing environment.

For example, a carbon capturing enzyme (e.g., a carbon fixing enzyme) provided herein comprising one or more non-standard amino acids, such as one or more selenocysteine residues, can capture or fix carbon in a buffer or environment with a redox potential of less than about −150 mV, with an activity that is at least 1.1, 1.2, 1.3, 1.4, 1.5, 1.6, 1.7, 1.8, 1.9, 2, 2.1, 2.2, 2.3, 2.4, 2.5, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20, 25, 30, 35, 40, 45, 50, 55, 60, 65, 70, 75, 80, 85, 90, 95, 100, 200, 300, 400, 500, 600, 700, 800, 900, or 1000 times higher than a corresponding carbon capturing enzyme (e.g., a carbon fixing enzyme) without the one or more non-standard amino acids in a buffer or environment with the same redox potential. For example, a carbon capturing enzyme (e.g., a carbon fixing enzyme) provided herein comprising one or more non-standard amino acids, such as one or more selenocysteine residues, can capture or fix carbon in a buffer or environment with a redox potential of less than about −160 mV, less than about −170 mV, less than about −180 mV, less than about −190 mV, less than about −200 mV, less than about −210 mV, less than about −220 mV, less than about −230 mV, less than about −240 mV, or less than about −250 mV, less than about −260 mV, less than about −270 mV, less than about −280 mV, less than about −290 mV, less than about −300 mV, less than about −310 mV, less than about −320 mV, less than about −330 mV, less than about −340 mV, or less than about −350 mV, less than about −360 mV, less than about −370 mV, less than about −380 mV, less than about −390 mV, less than about −400 mV, less than about −410 mV, less than about −420 mV, less than about −430 mV, less than about −440 mV, or less than about −450 mV, less than about −460 mV, less than about −470 mV, less than about −480 mV, less than about −490 mV, less than about −500 mV, less than about −510 mV, less than about −520 mV, less than about −530 mV, less than about −540 mV, or less than about −550 mV, less than about −560 mV, less than about −570 mV, less than about −580 mV, less than about −590 mV, or less than about −600 mV, with an activity that is at least 1.1, 1.2, 1.3, 1.4, 1.5, 1.6, 1.7, 1.8, 1.9, 2, 2.1, 2.2, 2.3, 2.4, 2.5, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20, 25, 30, 35, 40, 45, 50, 55, 60, 65, 70, 75, 80, 85, 90, 95, 100, 200, 300, 400, 500, 600, 700, 800, 900, or 1000 times higher than a corresponding carbon capturing enzyme (e.g., a carbon fixing enzyme) without the one or more non-standard amino acids in a buffer or environment with the same redox potential.

In some aspects, provided herein is a composition comprising a stabilized deoxyribonuclease I (DNase I) polypeptide comprising one or more non-standard amino acids, a functional fragment thereof, or a variant thereof. In some embodiments, the stabilized DNase I polypeptide may be made and used according to the invention with diselenide bonds between two selenocysteine residues. In some embodiments, the stabilized DNase I polypeptide can comprise one or more non-standard amino acids. In some embodiments, the stabilized DNase I polypeptide can comprise one or more selenocysteine residues. In some embodiments, the stabilized DNase I polypeptide can comprise a diselenide bond between two selenocysteine residues. The diselenide bonds may be intramolecular or intermolecular. In some embodiments, the stabilized DNase I polypeptide can comprise one or more diselenide bonds. In some embodiments, the stabilized DNase I polypeptide can comprise one or more catalytic selenocysteine substitutions.

In some aspects, provided herein is a composition comprising a stabilized deoxyribonuclease I (DNase I) polypeptide comprising one or more non-standard amino acids, a functional fragment thereof, or a variant thereof, wherein the stabilized DNase I polypeptide has a higher endonuclease activity for a DNA substrate in an environment than an endonuclease activity for the DNA substrate of a corresponding DNase I polypeptide, functional fragment thereof, or variant thereof that does not comprise the one or more non-standard amino acids. For example, the stabilized DNase I polypeptide can have at least 1.1, 1.2, 1.3, 1.4, 1.5, 1.6, 1.7, 1.8, 1.9, 2, 2.1, 2.2, 2.3, 2.4, 2.5, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20, 25, 30, 35, 40, 45, 50, 55, 60, 65, 70, 75, 80, 85, 90, 95, 100, 200, 300, 400, 500, 600, 700, 800, 900, or 1000 or greater fold higher endonuclease activity for a DNA substrate in an environment than an endonuclease activity for the DNA substrate of a corresponding DNase I polypeptide, functional fragment thereof, or variant thereof that does not comprise the one or more non-standard amino acids.

In some aspects, provided herein is a composition comprising a stabilized deoxyribonuclease I (DNase I) polypeptide comprising one or more non-standard amino acids, a functional fragment thereof, or a variant thereof, wherein the stabilized DNase I polypeptide does not destabilize in an environment that a corresponding DNase I polypeptide, functional fragment thereof, or variant thereof that does not comprise the one or more non-standard amino acids does destabilize. The destabilization can be obtained by contacting the corresponding DNase I polypeptide with one or more destabilization agents. The destabilization can be obtained by placing the corresponding DNase I polypeptide in a destabilization environment. The environment to destabilize the corresponding DNase I polypeptide is described elsewhere herein.

In some aspects, provided herein is a composition comprising a stabilized deoxyribonuclease I (DNase I) polypeptide comprising one or more non-standard amino acids, a functional fragment thereof, or a variant thereof, wherein the stabilized DNase I polypeptide has a melting temperature (Tₘ) that is at least 5° C. higher than a Tₘ of a corresponding DNase I polypeptide, functional fragment thereof, or variant thereof that does not comprise the one or more non-standard amino acids.

In some embodiments, the composition can comprise a stabilized deoxyribonuclease I (DNase I) polypeptide comprising one or more non-standard amino acids, a functional fragment thereof, or a variant thereof, wherein the stabilized DNase I polypeptide can have a melting temperature (Tₘ) that can be at least 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C. or 36° C. higher than a Tₘ of a corresponding DNase I polypeptide, functional fragment thereof, or variant thereof that does not comprise the one or more non-standard amino acids. In some embodiments, the composition can comprise a stabilized deoxyribonuclease I (DNase I) polypeptide comprising one or more non-standard amino acids, a functional fragment thereof, or a variant thereof, wherein the stabilized DNase I polypeptide can have a melting temperature (Tₘ) that can be less than 1° C. higher than a Tₘ of a corresponding DNase I polypeptide, functional fragment thereof, or variant thereof that does not comprise the one or more non-standard amino acids.

In some embodiments, at least one, two, three, four or more of the one or more non-standard amino acids is selenocysteine. In some embodiments, at least two of the one or more non-standard amino acids are directly linked by a bond.

In some embodiments, at least four of the one or more non-standard amino acids can be directly linked by a bond, wherein a first pair of the at least four of the one or more non-standard amino acids can be directly linked by a bond, and a second pair of at the least four of the one or more non-standard amino acids can be directly linked by a bond. In some embodiments, the bond is a diselenide bond. In some embodiments, the diselenide bond can be an intermolecular or an intramolecular bond. In some embodiments, the bond is a selenyl-sulfhydryl bond between a cysteine and a selenocysteine. In some embodiments, the selenyl-sulfhydryl bond can be an intermolecular or an intramolecular bond.

In some embodiments, the stabilized DNase I polypeptide, functional fragment thereof, or variant thereof, can have a half-life that can be at least a 1.1 fold higher than a half-life of a corresponding DNase I polypeptide, functional fragment thereof, or variant thereof that does not comprise the one or more non-standard amino acid. In some embodiments, the stabilized DNase I polypeptide, functional fragment thereof, or variant thereof, can have a half-life that can be at least a 1.1, 1.2, 1.3, 1.4, 1.5, 1.6, 1.7, 1.8, 1.9, 2, 2.1, 2.2, 2.3, 2.4, 2.5, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20, 25, 30, 35, 40, 45, 50, 55, 60, 65, 70, 75, 80, 85, 90, 95, 100, 200, 300, 400, 500, 600, 700, 800, 900, or 1000 or greater fold higher than a half-life of a corresponding DNase I polypeptide, functional fragment thereof, or variant thereof that does not comprise the one or more non-standard amino acid. In some embodiments, the stabilized DNase I polypeptide, functional fragment thereof, or variant thereof, can have a half-life that can be less than 1.1 fold higher than a half-life of a corresponding DNase I polypeptide, functional fragment thereof, or variant thereof that does not comprise the one or more non-standard amino acid.

In some embodiments, the stabilized DNase I polypeptide, functional fragment thereof, or variant thereof, can have at least 70%, 75%, 80%, 85%, 90%, 95% sequence identity to SEQ ID NO:1. In some embodiments, the stabilized DNase I polypeptide, functional fragment thereof, or variant thereof, can have at least 10%, 20%, 30%, 40%, 50%, 60%, 70%, 75%, 80%, 85%, 90%, 95% or greater sequence identity to SEQ ID NO:1. In some embodiments, the stabilized DNase I polypeptide, functional fragment thereof, or variant thereof, can have less than 10% sequence identity to SEQ ID NO:1.

In some embodiments, the stabilized DNase I polypeptide, functional fragment thereof, or variant thereof, can comprise a sequence with at least 70%, 75%, 80%, 85%, 90%, 95% sequence identity to at least 25, 50, 75, 100, 125, 150, 175, 200, 225, 250, or 261 contiguous amino acids of SEQ ID NO: 1. In some embodiments, the stabilized DNase I polypeptide, functional fragment thereof, or variant thereof, can comprise a sequence with at least 10%, 20%, 30%, 40%, 50%, 60%, 70%, 75%, 80%, 85%, 90%, 95% or greater sequence identity to at least 25, 50, 75, 100, 125, 150, 175, 200, 225, 250, or 261 contiguous amino acids of SEQ ID NO: 1. In some embodiments, the stabilized DNase I polypeptide, functional fragment thereof, or variant thereof, can comprise a sequence with less than 10% sequence identity to at least 25, 50, 75, 100, 125, 150, 175, 200, 225, 250, or 261 contiguous amino acids of SEQ ID NO: 1.

In some embodiments, the DNase I comprises an amino acid sequence with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to at least 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 260, 261, 262, 263, 264, 265, 266, 267, 268, 269, 270, 271, 272, 273, 274, 275, 276, 277, 278, 279, 280, 281, or 282 contiguous amino acids of SEQ ID NO: 1, where a non-standard amino acid such as selenocysteine is at positions 123, 126, 195, and/or 231.

In some embodiments, the DNase I further comprises at least one affinity tag. In some embodiments, an affinity tag of a DNase I is a C-terminal affinity tag. In some embodiments, an affinity tag of a DNase I is an N-terminal affinity tag. In some embodiments, a first affinity tag of a DNase I is an N-terminal affinity tag and a second affinity tag of a DNase I is a C-terminal affinity tag. In some embodiments, a first affinity tag of a DNase I is a first N-terminal affinity tag and a second affinity tag of a DNase I is a second N-terminal affinity tag. In some embodiments, a first affinity tag of a DNase I is a first C-terminal affinity tag and a second affinity tag of a DNase I is a second C-terminal affinity tag.

For example, the DNase I can comprise a poly-histidine tag, poly-histidine-glycine tag, poly-arginine tag, poly-aspartate tag, poly-cysteine tag, poly-phenylalanine, c-myc tag, Herpes simplex virus glycoprotein D (gD) tag, FLAG tag, KT3 epitope tag, tubulin epitope tag, T7 gene 10 protein peptide tag, streptavidin tag, streptavidin binding peptide (SPB) tag, Strep-tag, Strep-tag II, albumin-binding protein (ABP) tag, alkaline phosphatase (AP) tag, bluetongue virus tag (B-tag), calmodulin binding peptide (CBP) tag, chloramphenicol acetyl transferase (CAT) tag, choline-binding domain (CBD) tag, chitin binding domain (CBD) tag, cellulose binding domain (CBP) tag, dihydrofolate reductase (DHFR) tag, galactose-binding protein (GBP) tag, maltose binding protein (MBP), glutathione-S-transferase (GST), Glu-Glu (EE) tag, human influenza hemagglutinin (HA) tag, horseradish peroxidase (HRP) tag, NE-tag, HSV tag, ketosteroid isomerase (KSI) tag, KT3 tag, LacZ tag, luciferase tag, NusA tag, PDZ domain tag, AviTag, Calmodulin-tag, E-tag, S-tag, SBP-tag, Softag 1, Softag 3, TC tag, VSV-tag, Xpress tag, Isopeptag, SpyTag, SnoopTag, Profinity eXact tag, Protein C tag, S1-tag, S-tag, biotin-carboxy carrier protein (BCCP) tag, green fluorescent protein (GFP) tag, small ubiquitin-like modifier (SUMO) tag, tandem affinity purification (TAP) tag, HaloTag, Nus-tag, Thioredoxin-tag, Fc-tag, CYD tag, HPC tag, TrpE tag, ubiquitin tag, VSV-G epitope tag, V5 tag, or a combination thereof.

In some embodiments, the DNase I further comprises at least two affinity tags. For example, the DNase I can comprise at least two affinity tags selected from a poly-histidine tag, poly-histidine-glycine tag, poly-arginine tag, poly-aspartate tag, poly-cysteine tag, poly-phenylalanine, c-myc tag, Herpes simplex virus glycoprotein D (gD) tag, FLAG tag, KT3 epitope tag, tubulin epitope tag, T7 gene 10 protein peptide tag, streptavidin tag, streptavidin binding peptide (SPB) tag, Strep-tag, Strep-tag II, albumin-binding protein (ABP) tag, alkaline phosphatase (AP) tag, bluetongue virus tag (B-tag), calmodulin binding peptide (CBP) tag, chloramphenicol acetyl transferase (CAT) tag, choline-binding domain (CBD) tag, chitin binding domain (CBD) tag, cellulose binding domain (CBP) tag, dihydrofolate reductase (DHFR) tag, galactose-binding protein (GBP) tag, maltose binding protein (MBP), glutathione-S-transferase (GST), Glu-Glu (EE) tag, human influenza hemagglutinin (HA) tag, horseradish peroxidase (HRP) tag, NE-tag, HSV tag, ketosteroid isomerase (KSI) tag, KT3 tag, LacZ tag, luciferase tag, NusA tag, PDZ domain tag, AviTag, Calmodulin-tag, E-tag, S-tag, SBP-tag, Softag 1, Softag 3, TC tag, VSV-tag, Xpress tag, Isopeptag, SpyTag, SnoopTag, Profinity eXact tag, Protein C tag, S1-tag, S-tag, biotin-carboxy carrier protein (BCCP) tag, green fluorescent protein (GFP) tag, small ubiquitin-like modifier (SUMO) tag, tandem affinity purification (TAP) tag, HaloTag, Nus-tag, Thioredoxin-tag, Fc-tag, CYD tag, HPC tag, TrpE tag, ubiquitin tag, VSV-G epitope tag, and V5 tag.

In some embodiments, the DNase I comprises an affinity tag that is GST. In some embodiments, the DNase I comprises an affinity tag that is a poly-histidine tag, such as a 6×-His tag. In some embodiments, the DNase I comprises an affinity tag that is MBP. In some embodiments, the DNase I comprises an affinity tag that is a strep-tag, such as two strep tags.

In some embodiments, the DNase I comprises a first affinity tag that is GST and a second affinity tag that is a poly-histidine tag, such as a 6×-His tag. In some embodiments, the DNase I comprises a first affinity tag that is GST and a second affinity tag that is a strep tag. In some embodiments, the DNase I comprises a first affinity tag that is a strep tag, such as two strep tags, and a second affinity tag that is a poly-histidine tag, such as a 6×-His tag. In some embodiments, the DNase I comprises a first affinity tag that is MBP and a second affinity tag that is a poly-histidine tag, such as a 6×-His tag. In some embodiments, the DNase I comprises a first affinity tag that is MBP and a second affinity tag that is a strep tag, such as two strep tags.

In some embodiments, the DNase I comprises a first affinity tag that is GST, a second affinity tag that is a poly-histidine tag, such as a 6×-His tag, and a third affinity tag that is a strep tag, such as two strep tags. In some embodiments, the the DNase I comprises a GST tag, a His tag, and two strep tags. In some embodiments, the DNase I comprises a first affinity tag that is MBP, a second affinity tag that is a poly-histidine tag, such as a 6×-His tag, and a third affinity tag that is a strep tag, such as two strep tags. In some embodiments, the the DNase I comprises a MBP tag, a His tag, and two strep tags.

In some embodiments, the DNase I comprises an amino acid sequence with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any one of the SEQ ID NOs: 1 to 26.

In some embodiments, the DNase I comprises an affinity tag, wherein the DNase and affinity tag are separated by a linker. In some embodiments, the DNase I comprises a first affinity tag and a second affinity tag, wherein the DNase and the first affinity tag are separated by a linker, and wherein the DNase and the second affinity tag are separated by a linker. In some embodiments, the DNase I comprises a first affinity tag and a second affinity tag, wherein the first and second affinity tags are separated by a linker. In some embodiments, the DNase I comprises a first affinity tag, a second affinity tag and a third affinity tag, wherein the first, second and third affinity tags are each separated by a linker. In some embodiments, the DNase I comprises a first affinity tag, a second affinity tag, a third affinity tag and a fourth affinity tag, wherein the first, second, third and fourth affinity tags are each separated by a linker. In some embodiments, a linker comprises and amino acid sequence of (GS)n, (GGS)n, or (GGGS)n or a combination thereof, where n is an integer of rom 1-10.

In some embodiments, the one or more non-standard amino acids can be at position 123 of SEQ ID NO:1, position 126 of SEQ ID NO:1, position 195 of SEQ ID NO:1, or position 231 of SEQ ID NO:1. In some embodiments, the one or more non-standard amino acids can be at position 123 of SEQ ID NO:1, position 126 of SEQ ID NO:1, position 195 of SEQ ID NO:1, and position 231 of SEQ ID NO:1. In some embodiments, the one or more non-standard amino acids can be at position 123 of SEQ ID NO:1 and position 126 of SEQ ID NO:1. In some embodiments, the one or more non-standard amino acids can be at position 123 of SEQ ID NO:1 and position 195 of SEQ ID NO:1. In some embodiments, the one or more non-standard amino acids can be at position 123 of SEQ ID NO:1 and position 231 of SEQ ID NO:1. In some embodiments, the one or more non-standard amino acids can be at position 126 of SEQ ID NO:1 and position 195 of SEQ ID NO:1. In some embodiments, the one or more non-standard amino acids can be at position 126 of SEQ ID NO:1 and position 231 of SEQ ID NO:1. In some embodiments, the one or more non-standard amino acids can be at position 195 of SEQ ID NO:1 and position 231 of SEQ ID NO:1. In some embodiments, the one or more non-standard amino acids can be at position 123 of SEQ ID NO:1, position 126 of SEQ ID NO:1 and position 195 of SEQ ID NO:1. In some embodiments, the one or more non-standard amino acids can be at position 123 of SEQ ID NO:1, position 195 of SEQ ID NO:1, and position 231 of SEQ ID NO:1. In some embodiments, the one or more non-standard amino acids can be at position 123 of SEQ ID NO:1, position 105 of SEQ ID NO:1, and position 231 of SEQ ID NO:1. In some embodiments, the one or more non-standard amino acids can be at position 126 of SEQ ID NO:1, position 174 of SEQ ID NO:1, and position 231 of SEQ ID NO:1.

In some embodiments, the one or more non-standard amino acids can be at position 123 of SEQ ID NO:2, position 126 of SEQ ID NO:2, position 195 of SEQ ID NO:2, or position 231 of SEQ ID NO:2. In some embodiments, the one or more non-standard amino acids can be at position 123 of SEQ ID NO:2, position 126 of SEQ ID NO:2, position 195 of SEQ ID NO:2, and position 231 of SEQ ID NO:2.

In some embodiments, a non-standard amino acid at position 123 can be directly linked by a bond to a non-standard amino acid at position 126. In some embodiments, a non-standard amino acid at position 195 can be directly linked by a bond to a non-standard amino acid at position 231. In some embodiments, a non-standard amino acid at position 123 can be directly linked by a bond to a non-standard amino acid at position 195. In some embodiments, a non-standard amino acid at position 123 can be directly linked by a bond to a non-standard amino acid at position 231. In some embodiments, a non-standard amino acid at position 126 can be directly linked by a bond to a non-standard amino acid at position 195. In some embodiments, a non-standard amino acid at position 126 can be directly linked by a bond to a non-standard amino acid at position 231.

In some embodiments, the one or more non-standard amino acids can be at position 187 of SEQ ID NO:3 or position 224 of SEQ ID NO:3. In some embodiments, the one or more non-standard amino acids can be at position 187 of SEQ ID NO:3 and position 224 of SEQ ID NO:3. In some embodiments, a non-standard amino acid at position 187 of SEQ ID NO: 3 can be directly linked by a bond to a non-standard amino acid at position 224 of SEQ ID NO: 3.

In some embodiments, the bond can be a diselenide bond. In some embodiments, the diselenide bond can be in a location of a disulfide bond in a corresponding recombinant enzyme without the one or more non-standard amino acids.

In some embodiments, the Tₘ of the corresponding stabilized DNase I polypeptide, functional fragment thereof, or variant thereof, can be less than 37° C. In some embodiments, the Tₘ of the stabilized DNase I polypeptide, functional fragment thereof, or variant thereof, can be greater than 37° C., 40° C., 45° C., 50° C., 55° C., 60° C., or 65° C. In some embodiments, the Tₘ of the stabilized DNase I polypeptide, functional fragment thereof, or variant thereof, can be at least 37° C., 40° C., 45° C., 50° C., 55° C., 60° C., 65° C. or greater.

In some embodiments, the Tₘ of the stabilized DNase I polypeptide, functional fragment thereof, or variant thereof, can be at least 10° C. higher than the Tₘ of the corresponding recombinant enzyme, functional fragment thereof, or variant thereof. In some embodiments, the Tₘ of the stabilized DNase I polypeptide, functional fragment thereof, or variant thereof, can be at least 15° C. higher than the Tₘ of the corresponding recombinant enzyme, functional fragment thereof, or variant thereof. In some embodiments, the Tₘ of the stabilized DNase I polypeptide, functional fragment thereof, or variant thereof, can be at least 15° C., 20° C., 25° C., 30° C., 35° C., 40° C., 45° C., 50° C., 55° C., 60° C., 65° C. or greater higher than the Tₘ of the corresponding recombinant enzyme, functional fragment thereof, or variant thereof. In some embodiments, the Tₘ of the stabilized DNase I polypeptide, functional fragment thereof, or variant thereof, can be less than 10° C. higher than the Tₘ of the corresponding recombinant enzyme, functional fragment thereof, or variant thereof.

In some embodiments, the stabilized DNase I polypeptide, functional fragment thereof, or variant thereof, can have a half-life in an environment that can be at least a 1.1, 1.2, 1.3, 1.4, 1.5, 1.6, 1.7, 1.8, 1.9, 2, 2.1, 2.2, 2.3, 2.4, 2.5, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20, 25, 30, 35, 40, 45, 50, 55, 60, 65, 70, 75, 80, 85, 90, 95, 100, 200, 300, 400, 500, 600, 700, 800, 900, or 1000 or greater fold higher than a half-life of a corresponding DNase I polypeptide, functional fragment thereof, or variant thereof that does not comprise the one or more non-standard amino acids in the environment.

In some embodiments, the half-life of the DNase I polypeptide, functional fragment thereof, or variant thereof in the environment, can be greater than 1, 2, 3, 4, 5, 6, 7, 8, 9, 10, 11, 12, 13, 14, 15, 16, 17, 18, 19, 20, 21, 22, 23, 24 or more hours. In some embodiments, the half-life of the DNase I polypeptide, functional fragment thereof, or variant thereof in the environment, can be less than 1 hour.

In some embodiments, the half-life of the DNase I polypeptide, functional fragment thereof, or variant thereof in the environment, can be greater than 1, 2, 3, 4, 5, 6, 7, 8, 9, 10, 11, 12, 13, 14, 15, 16, 17, 18, 19, 20, 21, 22, 23, 24 or more days. In some embodiments, the half-life of the DNase I polypeptide, functional fragment thereof, or variant thereof in the environment, can be less than 1 day.

In some embodiments, the stabilized DNase I polypeptide can have at least a 1.1, 1.2, 1.3, 1.4, 1.5, 1.6, 1.7, 1.8, 1.9, 2, 3, 4, 5, 6, 7, 8, 10, 20, 30, 40, 50, 60, 70, 80, 90 or 100 fold higher endonuclease activity for a DNA substrate in an environment than an endonuclease activity for the DNA substrate of a corresponding DNase I polypeptide, functional fragment thereof, or variant thereof that does not comprise the one or more non-standard amino acids. In some embodiments, the stabilized DNase I polypeptide can have less than a 1.1 fold higher endonuclease activity for a DNA substrate in an environment than an endonuclease activity for the DNA substrate of a corresponding DNase I polypeptide, functional fragment thereof, or variant thereof that does not comprise the one or more non-standard amino acids.

In some embodiments, the stabilized DNase I polypeptide can have at least a 1.1, 1.2, 1.3, 1.4, 1.5, 1.6, 1.7, 1.8, 1.9, 2, 3, 4, 5, 6, 7, 8, 10, 20, 30, 40, 50, 60, 70, 80, 90 or 100 fold higher endonuclease activity for a DNA substrate after being present in an environment for at least 1, 2, 3, 4, 5, 6, 7, 8, 9, 10, 15, 20, 25, 30, 40, or 50 minutes than an endonuclease activity for the DNA substrate of a corresponding DNase I polypeptide, functional fragment thereof, or variant thereof that does not comprise the one or more non-standard amino acids after being present in the environment for at least 1, 2, 3, 4, 5, 6, 7, 8, 9, 10, 15, 20, 25, 30, 40, or 50 minutes. In some embodiments, the stabilized DNase I polypeptide can have less than a 1.1 fold higher endonuclease activity for a DNA substrate after being present in an environment for at least 1, 2, 3, 4, 5, 6, 7, 8, 9, 10, 15, 20, 25, 30, 40, or 50 minutes than an endonuclease activity for the DNA substrate of a corresponding DNase I polypeptide, functional fragment thereof, or variant thereof that does not comprise the one or more non-standard amino acids after being present in the environment for at least 1, 2, 3, 4, 5, 6, 7, 8, 9, 10, 15, 20, 25, 30, 40, or 50 minutes.

In some embodiments, the stabilized DNase I polypeptide can have at least a 1.1, 1.2, 1.3, 1.4, 1.5, 1.6, 1.7, 1.8, 1.9, 2, 3, 4, 5, 6, 7, 8, 10, 20, 30, 40, 50, 60, 70, 80, 90 or 100 fold higher endonuclease activity for a DNA substrate after being present in an environment for at least 1, 2, 3, 4, 5, 6, 7, 8, 9, 10, 12, 18, or 24 hours than an endonuclease activity for the DNA substrate of a corresponding DNase I polypeptide, functional fragment thereof, or variant thereof that does not comprise the one or more non-standard amino acids after being present in the environment for at least 1, 2, 3, 4, 5, 6, 7, 8, 9, 10, 12, 18, or 24 hours. In some embodiments, the stabilized DNase I polypeptide can have less than a 1.1 fold higher endonuclease activity for a DNA substrate after being present in an environment for at least 1, 2, 3, 4, 5, 6, 7, 8, 9, 10, 12, 18, or 24 hours than an endonuclease activity for the DNA substrate of a corresponding DNase I polypeptide, functional fragment thereof, or variant thereof that does not comprise the one or more non-standard amino acids after being present in the environment for at least 1, 2, 3, 4, 5, 6, 7, 8, 9, 10, 12, 18, or 24 hours.

In some embodiments, the DNA substrate can be genomic DNA. In some embodiments, the DNA substrate can be single-stranded DNA, double-stranded DNA, circular DNA, or cell free DNA.

In some embodiments, the environment can be an environment with a temperature of from 4° C.-98° C. In some embodiments, the environment can be an environment with a temperature of from 5° C.-97° C., 6° C.-96° C., 7° C.-95° C., 8° C.-94° C., 9° C.-93° C., 10° C.-92° C., 11° C.-91° C., 12° C.-90° C., 13° C.-89° C., 14° C.-88° C. 15° C.-85° C., 20° C.-75° C., 25° C.-70° C., 30° C.-65° C., 35° C.-60° C., 40° C.-55° C., or 45° C.-50° C. In some embodiments, the environment can be an environment with a temperature of at least 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 35° C., 40° C., 45° C., 50° C., 55° C., 60° C., 65° C., 70° C., 75° C., 80° C. or greater. In some embodiments, the environment can be an environment with a temperature of less than 4° C.

In some embodiments, the environment can be an environment with a lysis buffer. In some embodiments, the lysis buffer can be NP-40 lysis buffer, RIPA (RadiolmmunoPrecipitation Assay) lysis buffer, SDS (sodium dodecyl sulfate) lysis buffer, or ACK (Ammonium-Chloride-Potassium) lysing buffer.

In some embodiments, the environment can be an environment with a detergent at a concentration of from 0.01% to 20%. In some embodiments, the environment can be an environment with a detergent at a concentration of from 0.01% to 20%, 0.05% to 19.5%, 0.1% to 19%, 0.2% to 18.5%, 0.3% to 18%, 0.4% to 17.5%, 0.5% to 17%, 0.6% to 16.5%, 0.7% to 16%, 0.8% to 15%, 0.8% to 14%, 0.9% to 13%, or 1% to 12%. In some embodiments, the environment can be an environment with a detergent at a concentration of less than 0.01%. In some embodiments, the environment can be an environment with a detergent at a concentration of more than 20%.

In some embodiments, the detergent can be a non-ionic detergent. The non-ionic detergent can comprise Triton X-100, Triton X-114, NP-40, Brij-35, Brij-58, Tween 20, Tween 80, octyl glucoside, and octyl thioglucoside. In some embodiments, the detergent can be an ionic detergent. The ionic detergent can comprise sodium dodecyl sulfate (SDS). In some embodiment, the detergent can be a cationic detergent. The cationic detergent can be ethyl trimethyl ammonium bromide. In some embodiment, the detergent can be a zwitterionic detergent. The zwitterionic detergent can be CHAPS (3-[(3-cholamidopropyl)dimethylammonio]-1-propanesulfonate).

In some embodiments, the environment can comprise a divalent cation at a concentration of from 0.01 mM to 100 mM. In some embodiments, the environment can comprise a divalent cation at a concentration of from 0.01 mM to 100 mM, 0.05 mM to 95 mM, 0.1 mM to 90 mM, 0.5 mM to 85 mM, 1 mM to 80 mM, 5 mM to 75 mM, 10 mM to 70 mM, 15 mM to 65 mM, 20 mM to 60 mM, 25 mM to 55 mM, 30 mM to 50 mM, or 35 mM to 45 mM. In some embodiments, the environment can comprise a divalent cation at a concentration of less than 0.01 mM. In some embodiments, the environment can comprise a divalent cation at a concentration of more than 100 mM. In some embodiments, the divalent cation can be selected from the group consisting of Mg²⁺, Mn²⁺, Ca²⁺, Co²⁺, and Zn²⁺.

In some embodiments, the environment can comprise a reducing agent at a concentration of from 0.01 mM to 100 mM. The reducing agent can be glutathione, albumin, or thioredoxin. In some embodiments, the environment can comprise a reducing agent at a concentration of from 0.01 mM to 100 mM, 0.05 mM to 95 mM, 0.1 mM to 90 mM, 0.5 mM to 85 mM, 1 mM to 80 mM, 5 mM to 75 mM, 10 mM to 70 mM, 15 mM to 65 mM, 20 mM to 60 mM, 25 mM to 55 mM, 30 mM to 50 mM, or 35 mM to 45 mM. In some embodiments, the environment can comprise a reducing agent at a concentration of less than 0.01 mM. In some embodiments, the environment can comprise a reducing agent at a concentration of more than 100 mM. In some embodiments, the environment can comprise a reducing agent at a concentration of at least 200 mM, 300 mM, 400 mM, 500 mM, 600 mM, 700 mM, 800 mM, 900 mM, 1 M, or more.

In some embodiments, the environment can have a pH of from 5-9. In some embodiments, the environment have a pH of from 6-8. In some embodiments, the environment can have a pH of from 7-8. In some embodiments, the environment can have a pH of less than 5. In some embodiments, the environment can have a pH of more than 9.

In some embodiments, the environment can have a salt concentration of from 10 mM to 1 M. In some embodiments, the environment can have a salt concentration of from 15 mM to 950 mM, 20 mM to 900 mM, 30 mM to 850 mM, 40 mM to 800 mM, 50 mM to 750 mM, 60 mM to 700 mM, 70 mM to 650 mM, 80 mM to 600 mM, 90 mM to 550 mM, 100 mM to 500 mM, 150 mM to 450 mM, or 200 mM to 400 mM. In some embodiments, the environment can have a salt concentration of less than 10 mM. In some embodiments, the environment can have a salt concentration of more than 1 M.

In some embodiments, the environment can be within a droplet. In some embodiments, the environment can be a blood circulatory system. In some embodiments, the environment can be any environment where the stabilized DNase I polypeptide, functional fragment thereof, or variant thereof can have the enzyme activity.

In some embodiments, the environment can have a reduction potential that is less than −150 mV, −160 mV, −170 mV, −180 mV, −190 mV, −200 mV, −210 mV, −220 mV, −230 mV, −240 mV, or −250 mV, −260 mV, −270 mV, −280 mV, −290 mV, −300 mV, −310 mV, −320 mV, −330 mV, −340 mV, or −350 mV, −360 mV, −370 mV, −380 mV, −390 mV, −400 mV, −410 mV, −420 mV, −430 mV, −440 mV, or −450 mV, −460 mV, −470 mV, −480 mV, −490 mV, −500 mV, −510 mV, −520 mV, −530 mV, −540 mV, or −550 mV, −560 mV, −570 mV, −580 mV, −590 mV, or −600 mV. In some embodiments, the environment can have a reduction potential that is more than −150 mV.

In some embodiments, the stabilized DNase I polypeptide, functional fragment thereof, or variant thereof, can be recombinant. In some embodiments, the recombinant can be generated using recombinant DNA technology, such as, for example, the stabilized DNase I polypeptide, functional fragment thereof, or variant thereof expressed by a bacteriophage or yeast expression system. In other embodiments, the recombinant can be generated by the synthesis of a DNA molecule encoding the stabilized DNase I polypeptide, functional fragment thereof, or variant thereof.

In some embodiments, the stabilized DNase I polypeptide, functional fragment thereof, or variant thereof, can be bovine DNase I. The stabilized DNase I polypeptide, functional fragment thereof, or variant thereof, can be any other kinds of DNases I, including, but not limited to, E. coli DNase I, Microcella alkaliphila DNase I, Lactobacillus algidus DNase I, Vibrio cholerae DNase I, Bifidobacterium longum DNase I, Homo sapiens DNase I, and Raoultella ornithinolytica DNase I.

In some embodiments, a composition can comprise a polynucleotide encoding the composition disclosed herein. In some embodiments, the polynucleotide can be a vector. The vector can be a fragment of nucleic acid molecules. The vector can be taken from a virus, a plasmid, or the cell of a higher organism. The vector can be stably maintained in an organism. The vector can be inserted with a foreign nucleic acid fragment for cloning purposes. The vector can comprise features that allow for the convenient insertion or removal of a nucleic acid fragment to or from a vector. The vector can be genetically engineered plasmids.

In some embodiments, a bond directly linking two of the one or more non-standard amino acids of the stabilized DNase I polypeptide may not break in an environment, when the bond directly linking two of the one or more standard amino acids of the corresponding DNase I polypeptide may break in the same environment.

In some embodiments, the method of making the composition disclosed herein can comprise expressing an amino acid sequence of the stabilized DNase I polypeptide. In some embodiments, expressing can comprise expressing in a cell or in vitro.

In some embodiments, the cell can be a bacterial cell. In some embodiments, the cell can be a genomically recoded cell. In some embodiments, the cell may not be a bacterial cell. The cell can be obtained or isolated from a subject. The cell can be obtained or isolated from a tissue. The subject may be an animal such as a human, a mouse, a rat, a pig, a dog, a rabbit, a sheep, a horse, a chicken or other animal. A cell may be a neuron. The cell may be one of the cells of a blood-brain barrier system. The cell may be a cell line, such as a neuronal cell line. The cell may be a primary cell, such as cells obtained from a brain of a subject. The cell may be a population of cells that may be isolated from a subject, such as a tissue biopsy, a cytology specimen, a blood sample, a fine needle aspirate (FNA) sample, or any combination thereof. The cell may be obtained from a bodily fluid such as urine, milk, sweat, lymph, blood, sputum, amniotic fluid, aqueous humour, vitreous humour, bile, cerebrospinal fluid, chyle, chyme, exudates, endolymph, perilymph, gastric acid, mucus, pericardial fluid, peritoneal fluid, pleural fluid, pus, rheum, saliva, sebum, serous fluid, smegma, sputum, tears, vomit, or other bodily fluid. The cell may comprise cancerous cells, non-cancerous cells, tumor cells, non-tumor cells, healthy cells, or any combination thereof.

In some embodiments, the cell can comprise a reassigned codon recognized by a stabilizing non-standard amino acid tRNA comprising an anticodon corresponding to the reassigned codon.

In some embodiments, the amino acid sequence of the stabilized DNase I polypeptide can be encoded by a polynucleotide sequence comprising at least one codon of a natural amino acid that can have been replaced by the reassigned codon. In some embodiments, the amino acid sequence of the stabilized DNase I polypeptide can be encoded by a polynucleotide sequence comprising at least one, two, or three stop codons or codons of a natural amino acid that can be replaced by the reassigned codon.

In some embodiments, the stabilizing non-standard amino acid tRNA can be a selenocysteine tRNA.

In some embodiments, the method can comprise culturing the cell under conditions in which the amino acid sequence of the stabilized DNase I polypeptide can be expressed. In some embodiments, the reassigned codon can be UAG, UAA, UGA, or a combination thereof.

In some aspects, provided herein is a method comprising contacting DNA substrate that can be in a buffer, in reaction environment or on a solid surface to a stabilized deoxyribonuclease I (DNase I) polypeptide comprising one or more non-standard amino acids, a functional fragment thereof, or a variant thereof wherein the stabilized DNase I polypeptide, functional fragment thereof, or variant thereof can catalyze cleavage or fragmentation of the DNA substrate at a higher rate than a corresponding DNase I polypeptide, functional fragment thereof, or variant thereof that does not comprise the one or more non-standard amino acids. In some embodiments, the stabilized DNase I polypeptide, functional fragment thereof, or variant thereof can catalyze cleavage or fragmentation of the DNA substrate at a 1.1, 1.2, 1.3, 1.4, 1.5, 1.6, 1.7, 1.8, 1.9, 2, 2.1, 2.2, 2.3, 2.4, 2.5, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20, 25, 30, 35, 40, 45, 50, 55, 60, 65, 70, 75, 80, 85, 90, 95, 100, 200, 300, 400, 500, 600, 700, 800, 900, or 1000 or greater fold higher rate than a corresponding DNase I polypeptide, functional fragment thereof, or variant thereof that does not comprise the one or more non-standard amino acids.

In some embodiments, the stabilized DNase I polypeptide, functional fragment thereof, or variant thereof can be the stabilized DNase I polypeptide, functional fragment thereof, or variant thereof disclosed elsewhere herein. In some embodiments, the DNA substrate can be genomic DNA. In some embodiments, the DNA substrate is from a single cell. The single cell (or cell) is described elsewhere herein. In some embodiments, the method can comprise forming a plurality of vessels each comprising a single cell of a plurality of cells; the stabilized DNase I polypeptide, functional fragment thereof, or variant thereof and a lysis buffer. In some embodiments, the method can further comprise lysing the single cell, thereby releasing the DNA substrate from the single cell.

In some embodiments, the method can further comprise barcoding the DNA substrate or fragments thereof. The barcode on the DNA substrate can be a natural or synthetic nucleic acid sequence comprised by a polynucleotide allowing for unambiguous identification of the polynucleotide and other sequences comprised by the polynucleotide having said barcode sequence. The barcode may uniquely identify a subject, a sample (such as a cell-free sample), a nucleic acid sequence (such as a sequence having one or more epigenetically modified bases), or any combination thereof. The barcode may be associated with a DNA substrate or a complementary strand. The DNA substrate can comprise a single barcode. The DNA substrate may comprise one or more barcodes, such as a first barcode and a second barcode. In some cases, the first barcode can be different from the second barcode. In some cases, each barcode of a plurality of barcodes may be a unique barcode. In some cases, a barcode may comprise a sample identification barcode. For example, a first barcode may comprise a unique barcode and a second barcode may comprise a sample identification barcode.

In some embodiments, the method can further comprise amplifying the DNA substrate or fragments thereof. The amplification can comprise amplification by polymerase chain reaction (PCR), loop mediated isothermal amplification, nucleic acid sequence based amplification, strand displacement amplification, multiple displacement amplification, rolling circle amplification, ligase chain reaction, helicase dependent amplification, ramification amplification method, clonal amplification, or any combination thereof. In some cases, the amplification can comprise at least 2, 3, 4, 5, 6, 7, 8, 9, 10, 11, 12, 13 or greater cycles of amplification. In some embodiments, the amplifying can comprise clonal amplification. In some cases, individual DNA substrate or fragment can be amplified in situ on a support. In some cases, the amplification generates no more than about 10², 10³, 10⁴, 10⁵, 10⁶, 10⁷, 10⁸, 10⁹, 10¹⁰, 10¹¹, 10¹², 10¹⁵, or 10²⁰ amplicons from a single amplified template.

In some embodiments, the method further comprises sequencing the DNA substrate or fragments thereof. The sequencing can comprise bisulfite-free sequencing, bisulfite sequencing, TET-assisted bisulfite (TAB) sequencing, ACE-sequencing, high-throughput sequencing, Maxam-Gilbert sequencing, massively parallel signature sequencing, Polony sequencing, 454 pyrosequencing, Sanger sequencing, Illumina sequencing, SOLiD sequencing, Ion Torrent semiconductor sequencing, DNA nanoball sequencing, Heliscope single molecule sequencing, single molecule real time (SMRT) sequencing, nanopore DNA sequencing, shot gun sequencing, RNA sequencing, Enigma sequencing, or any combination thereof. In some embodiments, the sequencing comprises whole genome sequencing. In some embodiments, the sequencing comprises high throughput sequencing, massively parallel sequencing, Sanger sequencing, or next generation sequencing.

In some embodiments, the plurality of vessels comprises a solid support. In some embodiments, DNA substrate is not attached to the solid support in a vessel. In some embodiments, the DNA substrate can be attached to the solid support in a vessel. The support can be any solid or semisolid article on which reagents such as nucleic acids can be immobilized. Nucleic acids may be immobilized on the solid support by any method including but not limited to physical adsorption, by ionic or covalent bond formation, or combinations thereof. A solid support may include a polymeric, a glass, or a metallic material. Examples of solid supports include a membrane, a planar surface, a microtiter plate, a bead, a filter, a test strip, a slide, a cover slip, and a test tube, means any solid phase material upon which an oligomer is synthesized, attached, ligated or otherwise immobilized. The support may be composed of organic polymers such as polystyrene, polyethylene, polypropylene, polyfluoroethylene, polyethyleneoxy, and polyacrylamide, as well as co-polymers and grafts thereof. The support may also be inorganic, such as glass, silica, controlled-pore-glass (CPG), or reverse-phase silica. The configuration of a support may be in the form of beads, spheres, particles, granules, a gel, or a surface. Surfaces may be planar, substantially planar, or non-planar. Supports may be porous or non-porous, and may have swelling or non-swelling characteristics. The support can be shaped to comprise one or more wells, depressions or other containers, vessels, features or locations. A plurality of supports may be configured in an array at various locations.

In some embodiments, the buffer, the reaction environment or the solid surface can comprise primers specific to a sequence of the DNA substrate or fragments thereof. The primer may be a nucleic acid with known or unknown sequence. The primer may be single-stranded. In some cases, a primer can comprise a barcode (e.g. unique identifier sequence). The primer may be an amplification primer that hybridizes to the adapter and be extended using a target nucleic acid as a template in an amplification reaction. The primer can be a sequencing primer that hybridizes to the adapter and be extended using the target nucleic acid as a template in a sequencing reaction.

In some embodiments, the plurality of cells can comprise at least 2, 3, 4, 5, 5.5 6, 6.5 7, 8, 9, 10, 11, 12, 13, 14, 15, 16, 17, 18, 19, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10,000, 15,000, 20,000, 25,000, 30,000, 35,000, 40,000, 45,000, 50,000, 60,000, 70,000, 80,000, 90,000, 100,000, 200,000, 300,000, 400,000, 500,000, 600,000, 700,000, 800,000, 900,000, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², or 9×10¹² cells.

In some embodiments, the plurality of cells can be from one or more biological samples. The biological samples can be from a subject, such as a tissue biopsy, a cytology specimen, a blood sample, a fine needle aspirate (FNA) sample, or any combination thereof. The biological sample may be obtained from a bodily fluid such as urine, milk, sweat, lymph, blood, sputum, amniotic fluid, aqueous humour, vitreous humour, bile, cerebrospinal fluid, chyle, chyme, exudates, endolymph, perilymph, gastric acid, mucus, pericardial fluid, peritoneal fluid, pleural fluid, pus, rheum, saliva, sebum, serous fluid, smegma, sputum, tears, vomit, or other bodily fluid.

In some embodiments, the one or more biological samples comprises at least 2, 3, 4 5, 10, 20, 30, 40, 50, 60, 70, 80, 90 or 100 or more samples. In some embodiments, the one or more biological samples can comprise samples from different subjects. In some embodiments, the one or more biological samples can comprise samples from the same subject.

In some embodiments, the one or more biological sample is from a subject with a disease. The disease can include a cancer, a neurological disorder, or an autoimmune disease. In some embodiments, a disease may comprise a neurological disorder. In some cases, a neurological disorder may comprise Acquired Epileptiform Aphasia, Acute Disseminated Encephalomyelitis, Adrenoleukodystrophy, Agenesis of the corpus callosum, Agnosia, Aicardi syndrome, Alexander disease, Alpers' disease, Alternating hemiplegia, Alzheimer's disease, Amyotrophic lateral sclerosis (see Motor Neuron Disease), Anencephaly, Angelman syndrome, Angiomatosis, Anoxia, Aphasia, Apraxia, Arachnoid cysts, Arachnoiditis, Arnold-Chiari malformation, Arteriovenous malformation, Asperger's syndrome, Ataxia Telangiectasia, Attention Deficit Hyperactivity Disorder, Autism, Auditory processing disorder, Autonomic Dysfunction, Back Pain, Batten disease, Behcet's disease, Bell's palsy, Benign Essential Blepharospasm, Benign Focal Amyotrophy, Benign Intracranial Hypertension, Bilateral frontoparietal polymicrogyria, Binswanger's disease, Blepharospasm, Bloch-Sulzberger syndrome, Brachial plexus injury, Brain abscess, Brain damage, Brain injury, Brain tumor, Brown-Sequard syndrome, Canavan disease, Carpal tunnel syndrome (CTS), Causalgia, Central pain syndrome, Central pontine myelinolysis, Centronuclear myopathy, Cephalic disorder, Cerebral aneurysm, Cerebral arteriosclerosis, Cerebral atrophy, Cerebral gigantism, Cerebral palsy, Charcot-Marie-Tooth disease, Chiari malformation, Chorea, Chronic inflammatory demyelinating polyneuropathy (CIDP), Chronic pain, Chronic regional pain syndrome, Coffin Lowry syndrome, Coma, including Persistent Vegetative State, Congenital facial diplegia, Corticobasal degeneration, Cranial arteritis, Craniosynostosis, Creutzfeldt-Jakob disease, Cumulative trauma disorders, Cushing's syndrome, Cytomegalic inclusion body disease (CIBD), Cytomegalovirus Infection, Dandy-Walker syndrome, Dawson disease, De Morsier's syndrome, Dejerine-Klumpke palsy, Dejerine-Sottas disease, Delayed sleep phase syndrome, Dementia, Dermatomyositis, Neurological Dyspraxia, Diabetic neuropathy, Diffuse sclerosis, Dysautonomia, Dyscalculia, Dysgraphia, Dyslexia, Dystonia, Early infantile epileptic encephalopathy, Empty sella syndrome, Encephalitis, Encephalocele, Encephalotrigeminal angiomatosis, Encopresis, Epilepsy, Erb's palsy, Erythromelalgia, Essential tremor, Fabry's disease, Fahr's syndrome, Fainting, Familial spastic paralysis, Febrile seizures, Fisher syndrome, Friedreich's ataxia, FART Syndrome, Gaucher's disease, Gerstmann's syndrome, Giant cell arteritis, Giant cell inclusion disease, Globoid cell Leukodystrophy, Gray matter heterotopia, Guillain-Barre syndrome, HTLV-1 associated myelopathy, Hallervorden-Spatz disease, Head injury, Headache, Hemifacial Spasm, Hereditary Spastic Paraplegia, Heredopathia atactica polyneuritiformis, Herpes zoster oticus, Herpes zoster, Hirayama syndrome, Holoprosencephaly, Huntington's disease, Hydranencephaly, Hydrocephalus, Hypercortisolism, Hypoxia, Immune-Mediated encephalomyelitis, Inclusion body myositis, Incontinentia pigmenti, Infantile phytanic acid storage disease, Infantile Refsum disease, Infantile spasms, Inflammatory myopathy, Intracranial cyst, Intracranial hypertension, Joubert syndrome, Kearns-Sayre syndrome, Kennedy disease, Kinsboume syndrome, Klippel Feil syndrome, Krabbe disease, Kugelberg-Welander disease, Kuru, Lafora disease, Lambert-Eaton myasthenic syndrome, Landau-Kleffner syndrome, Lateral medullary (Wallenberg) syndrome, Learning disabilities, Leigh's disease, Lennox-Gastaut syndrome, Lesch-Nyhan syndrome, Leukodystrophy, Lewy body dementia, Lissencephaly, Locked-In syndrome, Lou Gehrig's disease, Lumbar disc disease, Lyme disease-Neurological Sequelae, Machado-Joseph disease (Spinocerebellar ataxia type 3), Macrencephaly, Maple Syrup Urine Disease, Megalencephaly, Melkersson-Rosenthal syndrome, Menieres disease, Meningitis, Menkes disease, Metachromatic leukodystrophy, Microcephaly, Migraine, Miller Fisher syndrome, Mini-Strokes, Mitochondrial Myopathies, Mobius syndrome, Monomelic amyotrophy, Motor Neuron Disease, Motor skills disorder, Moyamoya disease, Mucopolysaccharidoses, Multi-Infarct Dementia, Multifocal motor neuropathy, Multiple sclerosis, Multiple system atrophy, Muscular dystrophy, Myalgic encephalomyelitis, Myasthenia gravis, Myelinoclastic diffuse sclerosis, Myoclonic Encephalopathy of infants, Myoclonus, Myopathy, Myotubular myopathy, Myotonia congenita, Narcolepsy, Neurofibromatosis, Neuroleptic malignant syndrome, Neurological manifestations of AIDS, Neurological sequelae of lupus, Neuromyotonia, Neuronal ceroid lipofuscinosis, Neuronal migration disorders, Niemann-Pick disease, Non 24-hour sleep-wake syndrome, Nonverbal learning disorder, O'Sullivan-McLeod syndrome, Occipital Neuralgia, Occult Spinal Dysraphism Sequence, Ohtahara syndrome, Olivopontocerebellar atrophy, Opsoclonus myoclonus syndrome, Optic neuritis, Orthostatic Hypotension, Overuse syndrome, Palinopsia, Paresthesia, Parkinson's disease, Paramyotonia Congenita, Paraneoplastic diseases, Paroxysmal attacks, Parry-Romberg syndrome, Rombergs Syndrome, Pelizaeus-Merzbacher disease, Periodic Paralyses, Peripheral neuropathy, Persistent Vegetative State, Pervasive neurological disorders, Photic sneeze reflex, Phytanic Acid Storage disease, Pick's disease, Pinched Nerve, Pituitary Tumors, PMG, Polio, Polymicrogyria, Polymyositis, Porencephaly, Post-Polio syndrome, Postherpetic Neuralgia (PHN), Postinfectious Encephalomyelitis, Postural Hypotension, Prader-Willi syndrome, Primary Lateral Sclerosis, Prion diseases, Progressive Hemifacial Atrophy also known as Rombergs Syndrome, Progressive multifocal leukoencephalopathy, Progressive Sclerosing Poliodystrophy, Progressive Supranuclear Palsy, Pseudotumor cerebri, Ramsay-Hunt syndrome (Type I and Type II), Rasmussen's encephalitis, Reflex sympathetic dystrophy syndrome, Refsum disease, Repetitive motion disorders, Repetitive stress injury, Restless legs syndrome, Retrovirus-associated myelopathy, Rett syndrome, Reye's syndrome, Rombergs Syndrome, Rabies, Saint Vitus dance, Sandhoff disease, Schytsophrenia, Schilder's disease, Schizencephaly, Sensory Integration Dysfunction, Septo-optic dysplasia, Shaken baby syndrome, Shingles, Shy-Drager syndrome, Sjogren's syndrome, Sleep apnea, Sleeping sickness, Snatiation, Sotos syndrome, Spasticity, Spina bifida, Spinal cord injury, Spinal cord tumors, Spinal muscular atrophy, Spinal stenosis, Steele-Richardson-Olszewski syndrome, see Progressive Supranuclear Palsy, Spinocerebellar ataxia, Stiff-person syndrome, Stroke, Sturge-Weber syndrome, Subacute sclerosing panencephalitis, Subcortical arteriosclerotic encephalopathy, Superficial siderosis, Sydenham's chorea, Syncope, Synesthesia, Syringomyelia, Tardive dyskinesia, Tay-Sachs disease, Temporal arteritis, Tethered spinal cord syndrome, Thomsen disease, Thoracic outlet syndrome, Tic Douloureux, Todd's paralysis, Tourette syndrome, Transient ischemic attack, Transmissible spongiform encephalopathies, Transverse myelitis, Traumatic brain injury, Tremor, Trigeminal neuralgia, Tropical spastic paraparesis, Trypanosomiasis, Tuberous sclerosis, Vasculitis including temporal arteritis, Von Hippel-Lindau disease (VHL), Viliuisk Encephalomyelitis (VE), Wallenberg's syndrome, Werdnig-Hoffman disease, West syndrome, Whiplash, Williams syndrome, Wilson's disease, X-Linked Spinal and Bulbar Muscular Atrophy, and Zellweger syndrome.

In some cases, the disease may comprise an autoimmune disease. In some cases, an autoimmune disease may comprise acute disseminated encephalomyelitis (ADEM), acute necrotizing hemorrhagic leukoencephalitis, Addison's disease, agammaglobulinemia, allergic asthma, allergic rhinitis, alopecia areata, amyloidosis, ankylosing spondylitis, anti-GBM/anti-TBM nephritis, antiphospholipid syndrome (APS), autoimmune aplastic anemia, autoimmune dysautonomia, autoimmune hepatitius, autoimmune hyperlipidemia, autoimmune immunodeficiency, autoimmune inner ear disease (AIED), autoimmune myocarditis, autoimmune pancreatitis, autoimmune retinopathy, autoimmune thrombocytopenic purpura (ATP), autoimmune thyroid disease, axonal & neuronal neuropathies, Balo disease, Behcet's disease, bullous pemphigoid, cardiomyopathy, Castlemen disease, celiac sprue (non-tropical), Chagas disease, chronic fatigue syndrome, chronic inflammatory demyelinating polyneuropathy (CIDP), chronic recurrent multifocal ostomyelitis (CRMO), Churg-Strauss syndrome, cicatricial pemphigoid/benign mucosal pemphigoid, Crohn's disease, Cogan's syndrome, cold agglutinin disease, congenital heart block, coxsackie myocarditis, CREST disease, essential mixed cryoglobulinemia, demyelinating neuropathies, dermatomyositis, Devic's disease (neuromyelitis optica), discoid lupus, Dressler's syndrome, endometriosis, eosinophillic fasciitis, erythema nodosum, experimental allergic encephalomyelitis, Evan's syndrome, fibromyalgia, fibrosing alveolitis, giant cell arteritis (temporal arteritis), glomerulonephritis, Goodpasture's syndrome, Grave's disease, Guillain-Barre syndrome, Hashimoto's encephalitis, Hashimoto's thyroiditis, hemolytic anemia, Henock-Schoniein purpura, herpes gestationis, hypogammaglobulinemia, idiopathic thrombocytopenic purpura (ITP), IgA nephropathy, immunoregulatory lipoproteins, inclusion body myositis, insulin-dependent diabetes (type 1), interstitial cystitis, juvenile arthritis, juvenile diabetes, Kawasaki syndrome, Lambert-Eaton syndrome, leukocytoclastic vasculitis, lichen planus, lichen sclerosus, ligneous conjunctivitis, linear IgA disease (LAD), Lupus (SLE), Lyme disease, Meniere's disease, microscopic polyangitis, mixed connective tissue disease (MCTD), Mooren's ulcer, Mucha-Habermann disease, multiple sclerosis, myasthenia gravis, myositis, narcolepsy, neuromyelitis optica (Devic's), neutropenia, ocular cicatricial pemphigoid, optic neuritis, palindromic rheumatism, PANDAS (Pediatric Autoimmune Neuropsychiatric Disorders Associated with Streptococcus), paraneoplastic cerebellar degeneration, paroxysmal nocturnal hemoglobinuria (PNH), Parry Romberg syndrome, Parsonnage-Turner syndrome, pars plantis (peripheral uveitis), pemphigus, peripheral neuropathy, perivenous encephalomyelitis, pernicious anemia, POEMS syndrome, polyarteritis nodosa, type I, II & III autoimmune polyglandular syndromes, polymyalgia rheumatic, polymyositis, postmyocardial infarction syndrome, postpericardiotomy syndrome, progesterone dermatitis, primary biliary cirrhosis, primary sclerosing cholangitis, psoriasis, psoriatic arthritis, idiopathic pulmonary fibrosis, pyoderma gangrenosum, pure red cell aplasis, Raynaud's phenomena, reflex sympathetic dystrophy, Reiter's syndrome, relapsing polychondritis, restless legs syndrome, retroperitoneal fibrosis, rheumatic fever, rheumatoid arthritis, sarcoidosis, Schmidt syndrome, scleritis, scleroderma, Slogren's syndrome, sperm and testicular autoimmunity, stiff person syndrome, subacute bacterial endocarditis (SBE), sympathetic ophthalmia, Takayasu's arteritis, temporal arteritis/giant cell arteries, thrombocytopenic purpura (TPP), Tolosa-Hunt syndrome, transverse myelitis, ulcerative colitis, undifferentiated connective tissue disease (UCTD), uveitis, vasculitis, vesiculobullous dermatosis, vitiligo or Wegener's granulomatosis, chronic active hepatitis, primary biliary cirrhosis, cadilated cardiomyopathy, myocarditis, autoimmune polyendocrine syndrome type I (APS-I), cystic fibrosis vasculitides, acquired hypoparathyroidism, coronary artery disease, pemphigus foliaceus, pemphigus vulgaris, Rasmussen encephalitis, autoimmune gastritis, insulin hypoglycemic syndrome (Hirata disease), Type B insulin resistance, acanthosis, systemic lupus erythematosus (SLE), pernicious anemia, treatment-resistant Lyme arthritis, polyneuropathy, demyelinating diseases, atopic dermatitis, autoimmune hypothyroidism, vitiligo, thyroid associated ophthalmopathy, autoimmune coeliac disease, ACTH deficiency, dermatomyositis, Sjogren syndrome, systemic sclerosis, progressive systemic sclerosis, morphea, primary antiphospholipid syndrome, chronic idiopathic urticaria, connective tissue syndromes, necrotizing and crescentic glomerulonephritis (NCGN), systemic vasculitis, Raynaud syndrome, chronic liver disease, visceral leishmaniasis, autoimmune Cl deficiency, membrane proliferative glomerulonephritis (MPGN), prolonged coagulation time, immunodeficiency, atherosclerosis, neuronopathy, paraneoplastic pemphigus, paraneoplastic stiff man syndrome, paraneoplastic encephalomyelitis, subacute autonomic neuropathy, cancer-associated retinopathy, paraneoplastic opsoclonus myoclonus ataxia, lower motor neuron syndrome and Lambert-Eaton myasthenic syndrome.

In some cases, a disease may comprise AIDS, anthrax, botulism, brucellosis, chancroid, chlamydial infection, cholera, coccidioidomycosis, cryptosporidiosis, cyclosporiasis, dipheheria, ehrlichiosis, arboviral encephalitis, enterohemorrhagic Escherichia coli, giardiasis, gonorrhea, dengue fever, haemophilus influenza, Hansen's disease (Leprosy), hantavirus pulmonary syndrome, hemolytic uremic syndrome, hepatitis A, hepatitis B, hepatitis C, human immunodeficiency virus, legionellosis, listeriosis, lyme disease, malaria, measles. Meningococcal disease, mumps, pertussis (whooping cough), plague, paralytic poliomyelitis, psittacosis, Q fever, rabies, rocky mountain spotted fever, rubella, congenital rubella syndrome (SARS), shigellosis, smallpox, streptococcal disease (invasive group A), streptococcal toxic shock syndrome, Streptococcus pneumonia, syphilis, tetanus, toxic shock syndrome, trichinosis, tuberculosis, tularemia, typhoid fever, vancomycin intermediate resistant Staphylocossus aureus, varicella, yellow fever, variant Creutzfeldt-Jakob disease (vCJD), Eblola hemorrhagic fever, Echinococcosis, Hendra virus infection, human monkeypox, influenza A, H5N1, lassa fever, Margurg hemorrhagic fever, Nipah virus, O′nyong fever, Rift valley fever, Venezuelan equine encephalitis and West Nile virus.

In some cases, a disease may comprise a cancer. In some cases, a cancer may comprise thyroid cancer, adrenal cortical cancer, anal cancer, aplastic anemia, bile duct cancer, bladder cancer, bone cancer, bone metastasis, central nervous system (CNS) cancers, peripheral nervous system (PNS) cancers, breast cancer, Castleman's disease, cervical cancer, childhood Non-Hodgkin's lymphoma, lymphoma, colon and rectum cancer, endometrial cancer, esophagus cancer, Ewing's family of tumors (e.g. Ewing's sarcoma), eye cancer, gallbladder cancer, gastrointestinal carcinoid tumors, gastrointestinal stromal tumors, gestational trophoblastic disease, hairy cell leukemia, Hodgkin's disease, Kaposi's sarcoma, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, Non-Hodgkin's lymphoma, male breast cancer, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcoma (adult soft tissue cancer), melanoma skin cancer, non-melanoma skin cancer, stomach cancer, testicular cancer, thymus cancer, uterine cancer (e.g. uterine sarcoma), vaginal cancer, vulvar cancer, or Waldenstrom's macroglobulinemia.

In some cases, a disease can include hyperproliferative disorders. Malignant hyperproliferative disorders can be stratified into risk groups, such as a low risk group and a medium-to-high risk group. Hyperproliferative disorders can include but may not be limited to cancers, hyperplasia, or neoplasia. In some cases, the hyperproliferative cancer can be breast cancer such as a ductal carcinoma in duct tissue of a mammary gland, medullary carcinomas, colloid carcinomas, tubular carcinomas, and inflammatory breast cancer; ovarian cancer, including epithelial ovarian tumors such as adenocarcinoma in the ovary and an adenocarcinoma that has migrated from the ovary into the abdominal cavity; uterine cancer; cervical cancer such as adenocarcinoma in the cervix epithelial including squamous cell carcinoma and adenocarcinomas; prostate cancer, such as a prostate cancer selected from the following: an adenocarcinoma or an adenocarcinoma that has migrated to the bone; pancreatic cancer such as epithelioid carcinoma in the pancreatic duct tissue and an adenocarcinoma in a pancreatic duct; bladder cancer such as a transitional cell carcinoma in urinary bladder, urothelial carcinomas (transitional cell carcinomas), tumors in the urothelial cells that line the bladder, squamous cell carcinomas, adenocarcinomas, and small cell cancers; leukemia such as acute myeloid leukemia (AML), acute lymphocytic leukemia, chronic lymphocytic leukemia, chronic myeloid leukemia, hairy cell leukemia, myelodysplasia, myeloproliferative disorders, acute myelogenous leukemia (AML), chronic myelogenous leukemia (CML), mastocytosis, chronic lymphocytic leukemia (CLL), multiple myeloma (MM), and myelodysplastic syndrome (MDS); bone cancer; lung cancer such as non-small cell lung cancer (NSCLC), which may be divided into squamous cell carcinomas, adenocarcinomas, and large cell undifferentiated carcinomas, and small cell lung cancer; skin cancer such as basal cell carcinoma, melanoma, squamous cell carcinoma and actinic keratosis, which may be a skin condition that sometimes develops into squamous cell carcinoma; eye retinoblastoma; cutaneous or intraocular (eye) melanoma; primary liver cancer (cancer that begins in the liver); kidney cancer; autoimmune deficiency syndrome (AIDS)-related lymphoma such as diffuse large B-cell lymphoma, B-cell immunoblastic lymphoma and small non-cleaved cell lymphoma; Kaposi's Sarcoma; viral-induced cancers including hepatitis B virus (HBV), hepatitis C virus (HCV), and hepatocellular carcinoma; human lymphotropic virus-type 1 (HTLV-1) and adult T-cell leukemia/lymphoma; and human papilloma virus (HPV) and cervical cancer; central nervous system (CNS) cancers such as primary brain tumor, which includes gliomas (astrocytoma, anaplastic astrocytoma, or glioblastoma multiforme), oligodendrogliomas, ependymomas, meningiomas, lymphomas, schwannomas, and medulloblastomas; peripheral nervous system (PNS) cancers such as acoustic neuromas and malignant peripheral nerve sheath tumors (MPNST) including neurofibromas and schwannomas, malignant fibrous cytomas, malignant fibrous histiocytomas, malignant meningiomas, malignant mesotheliomas, and malignant mixed Müllerian tumors; oral cavity and oropharyngeal cancer such as hypopharyngeal cancer, laryngeal cancer, nasopharyngeal cancer, and oropharyngeal cancer; stomach cancer such as lymphomas, gastric stromal tumors, and carcinoid tumors; testicular cancer such as germ cell tumors (GCTs), which include seminomas and nonseminomas, and gonadal stromal tumors, which include Leydig cell tumors and Sertoli cell tumors; thymus cancer such as to thymomas, thymic carcinomas, Hodgkin disease, non-Hodgkin lymphomas carcinoids or carcinoid tumors; rectal cancer; and colon cancer. In some cases, the diseases stratified, classified, characterized, or diagnosed by the methods of the present disclosure include but may not be limited to thyroid disorders such as for example benign thyroid disorders including but not limited to follicular adenomas, Hurthle cell adenomas, lymphocytic thyroiditis, and thyroid hyperplasia. In some cases, the diseases stratified, classified, characterized, or diagnosed by the methods of the present disclosure include but may not be limited to malignant thyroid disorders such as for example follicular carcinomas, follicular variant of papillary thyroid carcinomas, medullary carcinomas, and papillary carcinomas.

The disease can include a genetic disorder. A genetic disorder may be an illness caused by abnormalities in genes or chromosomes. Genetic disorders can be grouped into two categories: single gene disorders and multifactorial and polygenic (complex) disorders. A single gene disorder can be the result of a single mutated gene. Inheriting a single gene disorder can include but not be limited to autosomal dominant, autosomal recessive, X-linked dominant, X-linked recessive, Y-linked and mitochondrial inheritance. Only one mutated copy of the gene can be necessary for a person to be affected by an autosomal dominant disorder. Examples of autosomal dominant type of disorder can include but may not be limited to Huntington's disease, Neurofibromatosis 1, Marfan Syndrome, Hereditary nonpolyposis colorectal cancer, or Hereditary multiple exostoses. In autosomal recessive disorders, two copies of the gene must be mutated for a subject to be affected by an autosomal recessive disorder. Examples of this type of disorder can include but may not be limited to cystic fibrosis, sickle-cell disease (also partial sickle-cell disease), Tay-Sachs disease, Niemann-Pick disease, or spinal muscular atrophy. X-linked dominant disorders are caused by mutations in genes on the X chromosome such as X-linked hypophosphatemic rickets. Some X-linked dominant conditions such as Rett syndrome, Incontinentia Pigmenti type 2 and Aicardi Syndrome can be fatal. X-linked recessive disorders are also caused by mutations in genes on the X chromosome. Examples of this type of disorder can include but are not limited to Hemophilia A, Duchenne muscular dystrophy, red-green color blindness, muscular dystrophy and Androgenetic alopecia. Y-linked disorders are caused by mutations on the Y chromosome. Examples can include but are not limited to Male Infertility and hypertrichosis pinnae. The genetic disorder of mitochondrial inheritance, also known as maternal inheritance, can apply to genes in mitochondrial DNA such as in Leber's Hereditary Optic Neuropathy.

In some embodiments, plurality of cells can comprise a plurality of bacterial cells or a plurality of fungal cells. In some embodiments, the plurality of bacterial cells or the plurality of fungal cells can comprise at least 2, 3, 4, 5, 5.5 6, 6.5 7, 8, 9, 10, 11, 12, 13, 14, 15, 16, 17, 18, 19, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10,000, 15,000, 20,000, 25,000, 30,000, 35,000, 40,000, 45,000, 50,000, 60,000, 70,000, 80,000, 90,000, 100,000, 200,000, 300,000, 400,000, 500,000, 600,000, 700,000, 800,000, 900,000, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², or 9×10¹² cells.

In some embodiments, plurality of cells comprises a plurality of immune cells. In some embodiments, the plurality of immune cells can comprise at least 2, 3, 4, 5, 5.5 6, 6.5 7, 8, 9, 10, 11, 12, 13, 14, 15, 16, 17, 18, 19, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10,000, 15,000, 20,000, 25,000, 30,000, 35,000, 40,000, 45,000, 50,000, 60,000, 70,000, 80,000, 90,000, 100,000, 200,000, 300,000, 400,000, 500,000, 600,000, 700,000, 800,000, 900,000, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², or 9×10¹² cells.

In some embodiments, plurality of cells comprises a plurality of diseased cells. In some embodiments, the plurality of diseased cells can comprise at least 2, 3, 4, 5, 5.5 6, 6.5 7, 8, 9, 10, 11, 12, 13, 14, 15, 16, 17, 18, 19, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10,000, 15,000, 20,000, 25,000, 30,000, 35,000, 40,000, 45,000, 50,000, 60,000, 70,000, 80,000, 90,000, 100,000, 200,000, 300,000, 400,000, 500,000, 600,000, 700,000, 800,000, 900,000, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1=10¹², 2×10¹², 3×10¹², 4×10¹², 5×10¹², 6×10¹², 7×10¹², 8×10¹², or 9×10¹² cells.

In some embodiments, plurality of cells comprises a plurality of cancer cells. In some embodiments, the plurality of cancer cells can comprise at least 2, 3, 4, 5, 5.5 6, 6.5 7, 8, 9, 10, 11, 12, 13, 14, 15, 16, 17, 18, 19, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10,000, 15,000, 20,000, 25,000, 30,000, 35,000, 40,000, 45,000, 50,000, 60,000, 70,000, 80,000, 90,000, 100,000, 200,000, 300,000, 400,000, 500,000, 600,000, 700,000, 800,000, 900,000, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², or 9×10¹² cells.

The enzymes described herein can be made in a host cell, or in vitro, in cell-free synthetic systems. Host cells may be any that can be robustly recoded. These can be bacterial cells that have well developed genetic systems, of which E. coli is exemplary. Other bacterial species can also be used. Cell-free systems for producing the proteins may be coupled transcription/translation systems or only translation systems. A notable aspect of the methods of the invention is the use of biological syntheses rather than chemical synthesis means.

Culturing of recoded cells with the constructed nucleic acid sequences may be by any means known in the art. The culturing may be batch or continuous, in shaker flasks or in fermenters or immobilized on solid surfaces, such as small particles contained in larger vessels. Typically the culture medium will be supplemented with a source of selenium, such as Na₂SeO₃. As is known in the art, production of the desired protein variant may be under the control of an inducer or a repressor. Any such systems which are known in the art may be selected for convenience of construction and protein production.

### III. Pharmaceutical Composition

### Compositions

The present disclosure relates to a pharmaceutical composition comprising (a) a stabilized DNase I polypeptide comprising one or more non-standard amino acids, a functional fragment thereof, or a variant thereof; and (b) at least one pharmaceutically acceptable carrier, excipient, or additive. The formulation and the pharmaceutically acceptable carrier, excipient, or additive should suit the routes of administration. In some embodiments, the pharmaceutical composition is suitable for a range of routes of administration including, without limitation, oral, topical, eye or ocular, parenteral, intramuscular, intravenous, subcutaneous, transdermal (which may include a penetration enhancement agent), buccal and suppository administration, and by inhalation. Pharmaceutically acceptable carriers, excipients, or additives include, but are not limited, to saline, buffered saline, dextrose, water (e.g., sterile water), glycerol, ethanol, sterile isotonic aqueous buffer, binding agent, and combinations thereof. In some embodiments, the at least one pharmaceutically acceptable carrier, excipient, or additive comprises saline or sterile water.

In some embodiments, the pharmaceutical composition is for treating a human subject with lung disease. In some embodiments, the human subject has a lung disease or is diagnosed with a lung disease. In some embodiments, the human subject has more than one lung disease. In some embodiments, the lung disease is a chronic disease or an acute disease. In some embodiments, the lung disease is one that affects the airways, one that affects the sacs (Alveoli), one that affects the interstitium, one that affects the pleura, or a combination thereof. In some embodiments, the lung disease leads to or is often associated with an overproduction of mucus or biofilm. In some embodiments, excessive production of airway mucus or biofilm is a feature of the lung disease.

In some embodiments, the lung disease is a pulmonary disease or condition. In some embodiments, the pulmonary disease or condition is an acute or chronic bronchopulmonary disease, atelectasis due to tracheal or bronchial impaction, or complications of tracheostomy. In some embodiments, the pulmonary disease or condition is acute or chronic bronchopulmonary disease, atelectasis due to tracheal or bronchial impaction and complications of tracheostomy chronic bronchitis, asthmatic bronchitis, CF, pneumonia, allergic diseases such as allergic asthma, non-allergic asthma, systemic lupus erythematosus, Sjogren's syndrome, bronchiectasis, emphysema, acute and chronic sinusitis, or conditions caused by the common cold. In some embodiments, the pulmonary disease or condition is infectious pneumonia, bronchitis or tracheobronchitis, bronchiectasis, CF, asthma, tuberculosis (TB), or fungal infections. In some embodiments, the lung disease is asthma (e.g., bronchial asthma), chronic obstructive pulmonary disease (COPD) (e.g., chronic bronchitis), bronchiectasis, acute bronchitis, or CF. In some embodiments, the lung disease is CF.

In some embodiments, the pharmaceutical composition further comprises one or more antibiotics. In some embodiments, the pharmaceutical composition comprises 1, 2, 3, 4, 5, or more antibiotics. In some embodiments, the pharmaceutical composition comprises antibiotics that are classified into 1, 2, 3, 4, 5, or more antibiotics classes. In some embodiments, the one or more antibiotics are selected from a group of antibiotics classes consisting of: Penicillins, Tetracyclines, Cephalosporins, Quinolones, Lincomycins, Macrolides, Sulfonamides, Glycopeptides, Aminoglycosides and Carbapenems. In some embodiments, the one or more antibiotics are effective against Pseudomonas aeruginosa, or any other bacteria that is associated with lung diseases. In some embodiments, the one or more antibiotics comprise amikacin, gentamicin, kanamycin, neomycin, netilmicin, tobramycin, paromomycin, streptomycin, ertapenem, doripenem, imipenem/cilastatin, meropenem, cefadroxil, cefazolin, cephradine, cephapirin, cephalothin, cefalexin, cefaclor, cefoxitin, cefotetan, cefamandole, cefmetazole, cefonicid, loracarbef, cefprozil, cefuroxime, cefixime, cefdinir, cefditoren, cefotaxime, cefpodoxime, ceftazidime, ceftibuten, ceftizoxime, moxalactam, ceftriaxone, cefepime, ceftaroline fosamil, ceftobiprole, clindamycin, lincomycin, daptomycin, telithromycin, aztreonam, penicillins, amoxicillin, ampicillin/sulbactam, piperacillin/tazobactam, ticarcillin/clavulanate, polypeptides, bacitracin, colistin, polymyxin b, ciprofloxacin, enoxacin, gatifloxacin, gemifloxacin, levofloxacin, lomefloxacin, moxifloxacin, nadifloxacin, nalidixic acid, norfloxacin, capreomycin, cycloserine, ethambutol(bs), ethionamide, isoniazid, pyrazinamide, rifampicin, rifabutin, rifapentine, or streptomycin. In some embodiments, the one or more antibiotics comprise amoxicillin, doxycycline, cephalexin, ciprofloxacin, clindamycin, metronidazole, azithromycin, sulfamethoxazole/trimethoprim, amoxicillin/clavulanate, levofloxacin, cefepime, tazobactam/piperacillin, meropenem, amikacin, gentamicin, aztreonam, colistin, or tobramycin. In some embodiments, the one or more antibiotics comprise an inhalable antibiotic. In some embodiments, the inhalable antibiotic is, for example, aztreonam, colistin, or tobramycin.

In some embodiments, the pharmaceutical composition further comprises glutathione (GSH). GSH is a naturally occurring tripeptide antioxidant that is capable of reducing disulfide bonds within a cytoplasmic protein to cysteines, by which process the GSH is oxidized into glutathione disulfide (GSSG). GSSH may then be reduced back to GSH by glutathione reductase. The ratio of GSH to GSSH in a cell is generally an indication of the oxidative stress of the cell. In some embodiments, the GSH is a pharmaceutical grade GSH. In some embodiments, the GSH is present in the pharmaceutical composition in an amount effective to enhance an efficiency of the one or more antibiotics. As used herein, an amount effective to enhance an efficiency of the one or more antibiotics may refer to an amount of GSH that improves the efficiency of the antibiotics in treating the lung disease. For example, in some embodiments, an effective amount of GSH reduces the dose of antibiotics by at least 2%, 5%, 10%, 15%, 20%, 30%, 40%, or 50% compared to the corresponding dose of antibiotics in the absence of the GSH. For another example, in some embodiments, an effective amount of GSH refers to an amount or concentration of GSH that reduces the minimal inhibitory concentration (MIC) of an antibiotic by at least 2%, 5%, 10%, 15%, 20%, 30%, 40%, or 50%, compared to the MIC of the antibiotic in the absence of the GSH. An MIC of an antibiotic is the lowest concentration of the antibiotic that inhibits a given bacterial isolation (e.g., Pseudomonas aeruginosa) from multiplying and producing visible growth in the test system. A standard method of determining the MIC of an antibiotic is provided by the American Society for Microbiology, as described in Manual of Antimicrobial Susceptibility Testing, Cavalieri et al. eds. 2005, which is hereby incorporated by reference in its entirety.

In some embodiments, the GSH is present in the pharmaceutical composition in an amount effective to reduce the GSH/GSSH redox potential in the human lung environment. The method of determining the GSH/GSSH redox potential in the lung environment has been described in the art, for example, as described in Yeh et al., Am J Respir Crit Care Med 2007 (176): 270-276, which is hereby incorporated by reference in its entirety. In some embodiments, the GSH is present in the pharmaceutical composition in an amount effective to shift the GSH/GSSH redox potential in the lung environment toward a reduced state. In certain embodiments, the GSH is present in the pharmaceutical composition in an amount that shifts the GSH/GSSH redox potential in a subject's bronchoalveolar lavage fluid (BALF) toward a reduced state by at least 10 mV, 20 mV, 30 mV, 40 mV, 50 mV, 60 mV, 70 mV, 80 mV, 90 mV, 100 mV, 110 mV, 120 mV, 130 mV, 140 mV, 150 mV, 160 mV, 170 mV, 180 mV, 190 mV, 200 mV, or 400 mV, compared to the GSH/GSSH redox potential in the BALF in the absence of the GSH. In certain embodiments, the GSH is present in the pharmaceutical composition in an amount that shifts the GSH/GSSH redox potential in a subject's BALF toward a reduced state by at least 1 mV, 2 mV, 5 mV, 10 mV, 15 mV, 20 mV, 25 mV, 30 mV, 35 mV, 40 mV, 45 mV, or 50 mV, compared to the GSH/GSSH redox potential in the BALF in the absence of the GSH.

### Pharmaceutical Compositions

The herein described pharmaceutical compositions may be formulated into different forms; for example, they may be formulated into a solid form (e.g., powders, tablets, capsule, creams, films, granules, paste, and gels) or a liquid form (e.g., suspension, solution, and syrup). In some embodiments, the pharmaceutical composition is formulated into a tablet, a capsule, a powder, which tablet, capsule or powder may or may not have sustained release characteristics.

In some embodiments, for solid dosage forms used in oral, the active ingredient is mixed with one or more pharmaceutically acceptable carriers, excipients, or additives, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents, in the case of capsules, tablets, and pills, the pharmaceutical compositions can also comprise buffering agents. Solid compositions of a similar type can also be prepared using fillers in soft and hard-filled gelatin capsules, and excipients such as lactose or milk sugars, as well as high molecular weight polyethylene glycols and the like. For oral administration, a pharmaceutically acceptable nontoxic composition is formed by incorporating any of the normally employed excipients, such as those carriers previously listed, and generally 10-95% of active ingredient, that is, one or more peptides of the invention, and for example, at a concentration of 25%-75%.

In some embodiments, the pharmaceutical composition is in a powder form. In some embodiments, the pharmaceutical composition is a dispersible powder. The pharmaceutically acceptable carrier, excipient, or additive suitable for formulating a dispersible powder includes, without limitation, sodium chloride, sugars (e.g., sucrose, lactose, trehalose and mannitol), and derived calcium or other divalent cations (e.g., those obtained from calcium chloride, zinc chloride, manganese chloride and magnesium chloride). In some embodiments, the dispersible powder has a mean particle size from about 0.2 to 50 μm, 0.2 to 25 μm, 0.2 to 15 μm, 0.2 to 10 μm, 0.5 to 10 μm, 1 to 10 μm. 1 to 8 μm, 1 to 6 μm, 1 to 5 μm, 1 to 4 μm, 1 to 3 μm, 2 to 10 μm, 2 to 8 μm, 2 to 6 μm, or 2 to 4 μm.

In some embodiments, the pharmaceutical composition is prepared by a method comprising (a) spray-drying a liquid composition comprising the stabilized DNase I polypeptide, functional fragment thereof, or variant thereof and the at least one pharmaceutically acceptable carrier, excipient, or additive, and (b) collecting the spray-dried product of step (a) as a dispersible powder containing the stabilized DNase I polypeptide, functional fragment thereof, or variant thereof. In some embodiments, the liquid composition comprises a carrier such as saline, water, or alcohol. In some embodiments, the liquid composition comprises sterile water. In some embodiments, the liquid composition comprises sodium chloride. In some embodiments, the liquid composition comprises a sugar.

In some embodiments, the liquid composition has a concentration of the stabilized DNase I polypeptide, functional fragment thereof, or variant thereof of about from 0.1 mg/ml to 400 mg/ml, 1 mg/ml to 300 mg/ml, 1 mg/ml to 200 mg/ml, 1 mg/ml to 100 mg/ml, 1 mg/ml to 80 mg/ml, 1 mg/ml to 50 mg/ml, 1 mg/ml to 25 mg/ml, 5 mg/ml to 100 mg/ml, 5 mg/ml to 50 mg/ml, or 5 mg/ml to 25 mg/ml. In some embodiments, the liquid composition has a concentration of the stabilized DNase I polypeptide, functional fragment thereof, or variant thereof of about from 1 mg/ml to 80 mg/ml. In some embodiments, the stabilized DNase I polypeptide, functional fragment thereof, or variant thereof is present in the dispersible powder in an amount of about 1% to 100% by weight. In some embodiments, the stabilized DNase I polypeptide, functional fragment thereof, or variant thereof is present in the dispersible powder in an amount of about at least 5%, 10%, 20%, 30%, 40%, 50%, 60%, 70%, 80%, 90%, or 95% by weight. In some embodiments, the stabilized DNase I polypeptide, functional fragment thereof, or variant thereof is present in the dispersible powder in an amount of about at most 10%, 20%, 30%, 40%, 50%, 60%, 70%, 80%, 90%, or 95% by weight. In some embodiments, the dispersible powder comprises a sugar in an amount of about from 10% to 90% by weight. In some embodiments, the dispersible powder comprises sodium chloride in an amount of about from 10% to 90% by weight.

In some embodiments, the pharmaceutical composition is prepared by a method further comprising combining GSH with the stabilized DNase I polypeptide, functional fragment thereof. For example, in some embodiments, the GSH may be combined with the dispersible powder that contains the stabilized DNase I polypeptide, functional fragment thereof. For another example, in some embodiments, the GSH may be combined with the stabilized DNase I polypeptide, functional fragment thereof to produce the liquid composition. For yet another example, in some embodiments, the GSH may be added into the liquid composition that comprises the stabilized DNase I polypeptide, functional fragment thereof.

In some embodiments, the pharmaceutical composition is prepared by a method further comprising combining one or more antibiotics with the stabilized DNase I polypeptide, functional fragment thereof. For example, in some embodiments, the one or more antibiotics may be combined with the dispersible powder that contains the stabilized DNase I polypeptide, functional fragment thereof. For another example, in some embodiments, the one or more antibiotics may be combined with the stabilized DNase I polypeptide, functional fragment thereof to produce the liquid composition. For yet another example, in some embodiments, the one or more antibiotics may be added into the liquid composition that comprises the stabilized DNase I polypeptide, functional fragment thereof.

In some embodiments, the pharmaceutical composition is in a liquid form such as suspension, solution, and syrup, which liquid form may or may not have a sustained release feature. In some embodiments, the pharmaceutical composition is an aerosolable solution or suspension. Ordinarily, an aqueous aerosol is made by formulating an aqueous solution or suspension of the active ingredient(s) together with conventional pharmaceutically-acceptable carriers and stabilizers. The carriers and stabilizers vary with the requirements of the particular compound, but typically include non-ionic surfactants (Tweens, Pluronics, or polyethylene glycol), innocuous proteins like serum albumin, sorbitan esters, oleic acid, lecithin, and amino acids such as glycine, buffers, salts, sugars or sugar alcohols. Aerosols generally are prepared from isotonic solutions.

A suitable carrier for the aerosolable solution or suspension may comprise, for example, saline, water, alcohol (e.g., ethanol), or a combination thereof. In some embodiments, the aerosolable solution or suspension comprises saline or sterile water. In certain embodiments, the aerosolable solution or suspension has a concentration of the stabilized DNase I polypeptide, functional fragment thereof, or variant thereof of about from 0.1 mg/ml to 50 mg/ml, 0.1 mg/ml to 20 mg/ml, 0.1 mg/ml to 10 mg/ml, 0.5 mg/ml to 10 mg/ml, 0.5 mg/ml to 5 mg/ml, 0.5 mg/ml to 2 mg/ml, or 0.75 mg/ml to 1.25 mg/ml. In some embodiments, the aerosolable solution or suspension has a concentration of the stabilized DNase I polypeptide, functional fragment thereof, or variant thereof of about at least 0.1 mg/ml, 0.25 mg/ml, 0.5 mg/ml, 0.75 mg/ml, 1 mg/ml, 1.25 mg/ml, 1.5 mg/ml, 2 mg/ml, 2.5 mg/ml, 3 mg/ml, 4 mg/ml, or 5 mg/ml. In some embodiments, the aerosolable solution or suspension has a concentration of the stabilized DNase I polypeptide, functional fragment thereof, or variant thereof of about at most 0.5 mg/ml, 0.75 mg/ml, 1 mg/ml, 1.25 mg/ml, 1.5 mg/ml, 2 mg/ml, 2.5 mg/ml, 3 mg/ml, 4 mg/ml, 5 mg/ml, 10 mg/ml, 20 mg/ml, or 50 mg/ml.

In some embodiments, the pharmaceutical composition is a dosage form and comprises 0.1-50 ml, 0.1-20 ml, 0.1-10 ml, 0.5-20 ml, 0.5-10 ml, 0.5-5 ml, 0.5-4 ml, 0.5-3 ml, 0.5-2 ml, 0.5-1.5 ml, 0.5-1 ml, 0.75-5 ml, 0.75-4 ml, 0.75-3 ml, 0.75-2.5 ml, 0.75-2 ml, 0.75-1.5 ml, 0.75-1.25 ml, or 0.75-1 ml. In some embodiments, the pharmaceutical composition is a dosage form and comprises at least about 0.1 ml, 0.25 ml, 0.5 ml, 0.75 ml, 1 ml, 1.25 ml, 1.5 ml, 1.75 ml, 2 ml, 3 ml, 4 ml, 5 ml, 6 ml, 7 ml, 8 ml, 9 ml, 10 ml, or 20 ml. In some embodiments, the pharmaceutical composition is a dosage form and comprises at most about 0.5 ml, 0.75 ml, 1 ml, 1.25 ml, 1.5 ml, 1.75 ml, 2 ml, 3 ml, 4 ml, 5 ml, 6 ml, 7 ml, 8 ml, 9 ml, 10 ml, 20 ml, or 50 ml.

In some embodiments, the pharmaceutical composition is a dosage form and the stabilized DNase I polypeptide, functional fragment thereof, or variant thereof is present in the dosage form at an amount of about 0.1 mg to 20 mg, 0.1 mg to 10 mg, 0.1 mg to 5 mg, 0.5 mg to 4 mg, 0.5 mg to 3 mg, 0.5 mg to 2.5 mg, 0.5 mg to 2 mg, 0.5 mg to 1.5 mg, 0.75 mg to 1.5 mg, 0.75 mg to 1.25 mg, or 1.5 mg to 2.5 mg. In some embodiments, the pharmaceutical composition is a dosage form and the stabilized DNase I polypeptide, functional fragment thereof, or variant thereof is present in the dosage form at an amount of at least about 0.1 mg, 0.25 mg, 0.5 mg, 0.75 mg, 1 mg, 1.25 mg, 1.5 mg, 2 mg, 3 mg, 4 mg, 5 mg, 6 mg, 7 mg, 8 mg, 9 mg, 10 mg, or 20 mg. In some embodiments, the pharmaceutical composition is a dosage form and the stabilized DNase I polypeptide, functional fragment thereof, or variant is present in the dosage form at an amount of at most 0.5 mg, 0.75 mg, 1 mg, 1.25 mg, 1.5 mg, 2 mg, 3 mg, 4 mg, 5 mg, 6 mg, 7 mg, 8 mg, 9 mg, 10 mg, 20 mg, or 50 mg.

In some embodiments, the aerosolable solution or suspension has a concentration of the GSH of about from 1 mg/ml to 800 mg/ml, 50 mg/ml to 400 mg/ml, 75 mg/ml to 300 mg/ml, 100 mg/ml to 200 mg/ml, 125 mg/ml to 175 mg/ml, 1 mg/ml to 200 mg/ml, 1 mg/ml to 100 mg/ml, 1 mg/ml to 50 mg/ml, or 1 mg/ml to 20 mg/ml. In some embodiments, the aerosolable solution or suspension has a concentration of the GSH of at least 1 mg/ml, 5 mg/ml, 10 mg/ml, 15 mg/ml, 20 mg/ml, 25 mg/ml, 50 mg/ml, 75 mg/ml, 100 mg/ml, 125 mg/ml, 150 mg/ml, 175 mg/ml, 200 mg/ml, 300 mg/ml, 500 mg/ml, or 800 mg/ml. In some embodiments, the aerosolable solution or suspension has a concentration of the GSH of at most 5 mg/ml, 10 mg/ml, 15 mg/ml, 20 mg/ml, 25 mg/ml, 50 mg/ml, 75 mg/ml, 100 mg/ml, 125 mg/ml, 150 mg/ml, 175 mg/ml, 200 mg/ml, 300 mg/ml, 500 mg/ml, or 800 mg/ml.

In some embodiments, the pharmaceutical composition is a dosage form and has an amount of the GSH of at least about 1 mg, 5 mg, 10 mg, 20 mg, 30 mg, 40 mg, 50 mg, 60 mg, 70 mg, 80 mg, 90 mg, 100 mg, 150 mg, 200 mg, 300 mg, 400 mg, 500 mg, 600 mg, 700 mg, 800 mg, or 1000 mg. In some embodiments, the pharmaceutical composition is a dosage form and has an amount of the GSH of at most about 5 mg, 10 mg, 20 mg, 30 mg, 40 mg, 50 mg, 60 mg, 70 mg, 80 mg, 90 mg, 100 mg, 150 mg, 200 mg, 300 mg, 400 mg, 500 mg, 600 mg, 700 mg, 800 mg, 1000 mg, or 1500 mg.

In certain embodiments, the pharmaceutical composition is a dosage form and has an amount that shifts the GSH/GSSH redox potential in a subject's bronchoalveolar lavage fluid (BALF) toward a reduced state by at least 10 mV, 20 mV, 30 mV, 40 mV, 50 mV, 60 mV, 70 mV, 80 mV, 90 mV, 100 mV, 110 mV, 120 mV, 130 mV, 140 mV, 150 mV, 160 mV, 170 mV, 180 mV, 190 mV, 200 mV, or 400 mV, compared to the GSH/GSSH redox potential in the BALF in the absence of the GSH. In some embodiments, the pharmaceutical composition is a dosage form and has an amount of the GSH that shifts the GSH/GSSH redox potential in a subject's BALF toward a reduced state by at least 1 mV, 2 mV, 5 mV, 10 mV, 15 mV, 20 mV, 25 mV, 30 mV, 35 mV, 40 mV, 45 mV, or 50 mV, compared to the GSH/GSSH redox potential in the subject's BALF in the absence of the GSH.

In some embodiments, a weight ratio of the GSH and the stabilized DNase I polypeptide, functional fragment thereof, or variant thereof present in the pharmaceutical composition for an administration is at least 6:1, 10:1, 25:1, 50:1, 100:1, 200:1, 300:1, 400:1, 500:1, 600:1, 800:1, 6000:1, or 60000:1. In some embodiments, a weight ratio of the GSH and the stabilized DNase I polypeptide, functional fragment thereof, or variant thereof present in the pharmaceutical composition for an administration is at most 6:1, 10:1, 25:1, 50:1, 100:1, 200:1, 300:1, 400:1, 500:1, 600:1, 800:1, 6000:1, or 60000:1. In some embodiments, a weight ratio of the GSH and the stabilized DNase I polypeptide, functional fragment thereof, or variant thereof present in the pharmaceutical composition for an administration is about from 200:1 to 1000:1, 400:1 to 800:1, or 500:1 to 700:1.

### IV. Method for Treatment

Provided herein is a method of treating a human subject with a lung disease comprising administering to the human subject a stabilized deoxyribonuclease I (DNase I) polypeptide comprising one or more non-standard amino acids, a functional fragment thereof, or a variant thereof. Provided herein is a method of loosening the mucus in the airways of a human subject with a lung disease, thereby treating the lung disease. In some embodiments, the method comprises delivering the pharmaceutical composition to the airways of the subject. In some embodiments, the method comprises depositing at least a portion of the pharmaceutical composition to the airways (e.g., lungs) of the subject. In some embodiments, the method comprises contacting the pharmaceutical composition to the biofilm of mucus in the airways of the subject.

In some embodiments, the method further comprises administering to the subject one or more antibiotics. In some embodiments, the method further comprises administering to the subject GSH. In some embodiments, the method comprises administering to the subject an effective amount of GSH to enhance an efficiency of the one or more antibiotics. In some embodiments, the method comprises administering to the subject GSH and one or more antibiotics.

In some embodiments, the method comprises administering the pharmaceutical composition orally, by inhalation, by nasal administration, or parenterally. The term parenteral as used herein includes, without limitation, subcutaneous, intravenous, intramuscular, intrasternal, intraperitoneal, and infusion techniques. In some embodiments, the method comprises administering the pharmaceutical composition by 1, 2, or more administration routes; for example, one component of the pharmaceutical composition is administered by inhalation and another component is administered orally or by injection. In some embodiments, the stabilized DNase I polypeptide comprising one or more non-standard amino acids, a functional fragment thereof, or a variant thereof is administered by inhalation, and the GSH is administered orally. In some embodiments, the stabilized DNase I polypeptide comprising one or more non-standard amino acids, a functional fragment thereof, or a variant thereof is administered by inhalation, and the one or more antibiotics are administered orally. In some embodiments, the stabilized DNase I polypeptide comprising one or more non-standard amino acids, a functional fragment thereof, or a variant thereof is administered by inhalation or orally, and the one or more antibiotics are administered parenterally. In some embodiments, all the components of the pharmaceutical composition are administered by the same route of administration, such as by inhalation.

Accordingly, in some embodiments, the method comprises administering to the human subject via inhalation a pharmaceutical composition comprising the stabilized DNase I polypeptide, functional fragment thereof, or variant thereof. In some embodiments, the method comprises administering to the human subject via inhalation a dispersible powder that comprises the stabilized DNase I polypeptide comprising one or more non-standard amino acids, a functional fragment thereof, or a variant thereof. In some embodiments, the method comprises administering to the human subject via inhalation a liquid formulation that comprises the stabilized DNase I polypeptide comprising one or more non-standard amino acids, a functional fragment thereof, or a variant thereof. In some embodiments, the method comprises administering to the human subject via inhalation an aerosolable solution or suspension that comprises the stabilized DNase I polypeptide comprising one or more non-standard amino acids, a functional fragment thereof, or a variant thereof.

The method of treatment may comprise delivering the pharmaceutical composition by any suitable means; for example, for administration by inhalation, the pharmaceutical composition may be delivered via a nebulizer, a pressurized metered-dose inhaler, or a dry powder inhaler. Methods of delivering drugs via inhalation are known in the art (e.g., see Ibrahim et al., Medical Devices: Evidence and Research, 2015:8 131-139 and Patton et al., Nature Reviews: Drug Discovery, February 2007:6, 67-74, which are hereby incorporated by reference in their entirety). In some embodiments, the method comprises delivering a dispersible powder via a dry powder inhaler. In other embodiments, the method comprises delivering an aerosolable solution or suspension via a nebulizer. In further embodiments, the method comprises delivering a liquid formulation via a pressurized metered-dose inhaler.

In certain embodiments, the pharmaceutical composition is delivered as an aerosol. In some embodiments, the aerosol has a predetermined mass medial aerodynamic diameter (MMAD) from about 0.5 μm to 10 μm, 0.5 μm to 5 μm, 1 μm to 8 μm, 1 μm to 6 μm, 2 μm to 6 μm, 2 μm to 5 μm, or 2 μm to 4 μm. In some embodiments, the aerosol has an MMAD of at least about 0.5 μm, 0.75 μm, 1 μm, 1.25 μm, 1.5 μm, 1.75 μm, 2 μm, 2.5 μm, 3 μm, 4 μm, 5 μm, or 6 μm. In some embodiments, the aerosol has an MMAD of at most about 1 μm, 1.25 μm, 1.5 μm, 1.75 μm, 2 μm, 2.5 μm, 3 μm, 4 μm, 5 μm, 6 μm, 7 μm, 8 μm, 9 μm, or 10 μm.

In some embodiments, the stabilized DNase polypeptide, functional fragment thereof, or variant thereof is present in the pharmaceutical composition or in the aerosol at a concentration of about 0.1 mg/ml to 50 mg/ml, 0.1 mg/ml to 20 mg/ml, 0.1 mg/ml to 10 mg/ml, 0.5 mg/ml to 10 mg/ml, 0.5 mg/ml to 5 mg/ml, 0.5 mg/ml to 2 mg/ml, or 0.75 mg/ml to 1.25 mg/ml. In some embodiments, the stabilized DNase polypeptide, functional fragment thereof, or variant thereof is present in the pharmaceutical composition or in the aerosol at a concentration of about at least 0.1 mg/ml, 0.25 mg/ml, 0.5 mg/ml, 0.75 mg/ml, 1 mg/ml, 1.25 mg/ml, 1.5 mg/ml, 2 mg/ml, 2.5 mg/ml, 3 mg/ml, 4 mg/ml, or 5 mg/ml. In some embodiments, the stabilized DNase polypeptide, functional fragment thereof, or variant thereof is present in the pharmaceutical composition or in the aerosol at a concentration of about at most 0.5 mg/ml, 0.75 mg/ml, 1 mg/ml, 1.25 mg/ml, 1.5 mg/ml, 2 mg/ml, 2.5 mg/ml, 3 mg/ml, 4 mg/ml, 5 mg/ml, 10 mg/ml, 20 mg/ml, or 50 mg/ml.

In some embodiments, about from 0.1-50 ml, 0.1-20 ml, 0.1-10 ml, 0.5-20 ml, 0.5-10 ml, 0.5-5 ml, 0.5-4 ml, 0.5-3 ml, 0.5-2 ml, 0.5-1.5 ml, 0.5-1 ml, 0.75-5 ml, 0.75-4 ml, 0.75-3 ml, 0.75-2.5 ml, 0.75-2 ml, 0.75-1.5 ml, 0.75-1.25 ml, or 0.75-1 ml of the aerosol is administered. In some embodiments, at least about 0.1 ml, 0.25 ml, 0.5 ml, 0.75 ml, 1 ml, 1.25 ml, 1.5 ml, 1.75 ml, 2 ml, 3 ml, 4 ml, 5 ml, 6 ml, 7 ml, 8 ml, 9 ml, 10 ml, or 20 ml of the aerosol is administered. In some embodiments, at most about 0.5 ml, 0.75 ml, 1 ml, 1.25 ml, 1.5 ml, 1.75 ml, 2 ml, 3 ml, 4 ml, 5 ml, 6 ml, 7 ml, 8 ml, 9 ml, 10 ml, 20 ml, or 50 ml of the aerosol is administered.

In some embodiments, the method comprises delivering the aerosol to the human subject via a nebulizer such as a jet nebulizer or an ultrasonic nebulizer. In some embodiments, the aerosol is delivered to the human subject within a period of time for each administration, for example, within 30 minutes, 20 minutes, 15 minutes, 10 minutes, 9 minutes, 8 minutes, 7 minutes, 6 minutes, 5 minutes, 4 minutes, 3 minutes, 2 minutes, or 1 minute.

The schedule of administration for the present method of treatment may depend on many factors such as the subject's condition and the severity of the disease. A care provider of the subject may determine a suitable dose and schedule for the subject. In some embodiments, the method comprises administering the pharmaceutical composition to the human subject for a period time. In other embodiments, the method comprises administering the pharmaceutical composition to the human subject for his or her life time. In some embodiments, the pharmaceutical composition is administered over a period of at least 1 day, 1 week, 1 month, 6 months, 1 year, 2 years, 5 years, or 10 years. In some embodiments, the pharmaceutical composition is administered over a period of at most 1 week, 1 month, 6 months, 1 year, 2 years, 5 years, 10 years, or 30 years.

In certain embodiments, the aerosol or the pharmaceutical composition comprising the stabilized DNase polypeptide, functional fragment thereof, or variant thereof is administered 1, 2, 3, 4, 5, or more times a day or 1, 2, 3, 4, 5, or more times a week. In further embodiments, the aerosol or the pharmaceutical composition comprising the stabilized DNase polypeptide, functional fragment thereof, or variant thereof is administered every 6 hours, 12 hours, 24 hours, or 48 hours. In some embodiments, the aerosol or the pharmaceutical composition comprising the stabilized DNase polypeptide, functional fragment thereof, or variant thereof is administered at least 1 time, 2 times, 3 times, or 4 times a day. In some embodiments, the aerosol or the pharmaceutical composition comprising the stabilized DNase polypeptide, functional fragment thereof, or variant thereof is administered at most 1 time, 2 times, 3 times, or 4 times a day.

In some embodiments, the stabilized DNase polypeptide, functional fragment thereof, or variant thereof, the GSH, and the one or more antibiotics are administered simultaneously. In some embodiments, simultaneous administrations of the stabilized DNase polypeptide, functional fragment thereof, or variant thereof, the GSH, and the one or more antibiotics are performed via the same route of administration; for example, all of them are administered via inhalation. In some embodiments, simultaneous administrations of the stabilized DNase polypeptide, functional fragment thereof, or variant thereof, the GSH, and the one or more antibiotics are performed via more than one route of administration.

In other embodiments, the stabilized DNase polypeptide, functional fragment thereof, or variant thereof, the GSH, and the one or more antibiotics are administered sequentially. In some embodiments, the stabilized DNase polypeptide, functional fragment thereof, or variant thereof is administered before the administration of the GSH and/or the antibiotics. In some embodiments, the GSH is administered before the administration of the antibiotics or the stabilized DNase polypeptide, functional fragment thereof, or variant thereof. In some embodiments, the one or more antibiotics are administered before the administration of the GSH or the stabilized DNase polypeptide, functional fragment thereof, or variant thereof. In some embodiments, the stabilized DNase polypeptide, functional fragment thereof, or variant thereof, the GSH, and the one or more antibiotics are separately administered within 1 hour, 2 hours, 6 hours, 12 hours, 1 day, or 1 week.

### V. Method of Disrupting Biofilm

Provided herein is a method of disrupting a biofilm comprising contacting a stabilized DNase I polypeptide comprising one or more non-standard amino acids, a functional fragment thereof, or a variant thereof, to the biofilm. In some embodiments, the method comprises contacting the pharmaceutical composition to the biofilm. In some embodiments, the method comprises contacting the GSH to the biofilm. In some embodiments, the method comprises contacting the one or more antibiotics to the biofilm. In some embodiments, the method comprises contacting an effective amount of the GSH to the biofilm to enhance an efficiency of the one or more antibiotics. In some embodiments, the method comprises contacting the pharmaceutical composition comprising the GSH, the one or more antibiotics and the stabilized DNase I polypeptide comprising one or more non-standard amino acids, a functional fragment thereof, or a variant thereof, to the biofilm.

In some embodiments, the method comprises contacting the GSH, the one or more antibiotics and the stabilized DNase I polypeptide comprising one or more non-standard amino acids, a functional fragment thereof, or a variant thereof, to the biofilm at the same or different time. In some embodiments, the contact of the GSH, the one or more antibiotics, and the stabilized DNase I polypeptide comprising one or more non-standard amino acids, a functional fragment thereof, or a variant thereof to the biofilm occur simultaneous or sequentially. In some specific embodiments, a portion of the biofilm is contacted with one component of the pharmaceutical composition and a different portion of the biofilm is contacted with another component of the pharmaceutical composition.

In some embodiments, the biofilm is found on an environmental surface or on the exterior or interior surface of a medical device. In certain embodiments, the environmental surface or the surface of a medical device is a surface of a medical implant, surface of surgical and hospital environment, surface of home and hospital bathrooms, surface of air-handling or water-handling systems, surface of biological fluid-handling machines, or surface of catheters.

In some embodiments, the biofilm is found on chronic wounds (e.g., diabetic ulcers), infections (e.g., ear infections), or diseases (lung diseases such as CF). In some embodiments, the biofilm is found in the respiratory tract of a human subject having a lung disease.

### V. Kits and Co-Packaging

Described herein is a kit containing any one or more of the components discussed herein to allow administration of the pharmaceutical composition or the implementation of the method. Component may be provided individually or in combinations, and may be provided in any suitable containers, such as a vial, a bottle, a tube, an ampule, or a pouch. In some embodiments, the kit includes instructions in one or more languages, for example in more than one language. In some embodiments, a kit comprises one or more reagents for use in a process utilizing one or more of the components described herein. Reagents may be provided in any suitable container. For example, a kit may provide one or more delivery or storage buffers. For another example, a kit may provide one or more carriers such as sterile water and saline. Reagents may be provided in a form that is usable in a particular process, or in a form that requires addition of one or more other components before use (e.g. in concentrate or lyophilized form). The kit may advantageously provide all components of the described method or composition. In some embodiments, the kit comprises the GSH, the one or more antibiotics, and the stabilized DNase I polypeptide comprising one or more non-standard amino acids, a functional fragment thereof, or a variant thereof.

In some embodiments, the kit comprises a component comprising a stabilized DNase I polypeptide comprising one or more non-standard amino acids, a functional fragment thereof, or a variant thereof. In some embodiments, the kit comprises a component comprising one or more antibiotics. In some embodiments, the kit comprises a component comprising GSH. In some embodiments, the kit comprises a component comprising an effective amount of GSH to enhance the efficiency of the one or more antibiotics. In some embodiments, the kit comprises a first component comprising a stabilized DNase I polypeptide comprising one or more non-standard amino acids, a functional fragment thereof, or a variant thereof, and a second component comprising one or more antibiotics. In some embodiments, the kit comprises a first component comprising a stabilized DNase I polypeptide comprising one or more non-standard amino acids, a functional fragment thereof, or a variant thereof, and a second component comprising GSH. In some embodiments, the kit comprises a first component comprising a stabilized DNase I polypeptide comprising one or more non-standard amino acids, a functional fragment thereof, or a variant thereof, a second component comprising one or more antibiotics, and a third component comprising GSH.

In some embodiments, each component of the kit is independently in a solid form (e.g., dispersible powder, tablet, capsule, and granules) or liquid form (e.g., aerosolable solution or suspension). In some embodiments, the one or more components in the kit are in the same form, e.g., in liquid form or in solid form. In some embodiments, the one or more components in the kit are in different forms, e.g., one component in liquid form and another component in solid form. For example, in some embodiments, both the stabilized DNase I polypeptide comprising one or more non-standard amino acids, a functional fragment thereof, or a variant thereof and the GSH are in liquid form, e.g., aerosolable solution or suspension. For another example, in some embodiments, both the stabilized DNase I polypeptide comprising one or more non-standard amino acids, a functional fragment thereof, or a variant thereof and the GSH are in liquid form, and the one or more antibiotics are in solid form.

In some embodiments, the kit comprises a solution that contains all or a portion of the stabilized DNase I polypeptide comprising one or more non-standard amino acids, a functional fragment thereof, or a variant thereof. In some embodiments, the kit comprises lyophilized form of the stabilized DNase I polypeptide comprising one or more non-standard amino acids, a functional fragment thereof, or a variant thereof. In some embodiments, the kit comprises a carrier such as sterile water or saline to be combined with one or more component of the kits; for example, a kit may contain sterile water to be combined with the lyophilized stabilized DNase I polypeptide comprising one or more non-standard amino acids, a functional fragment thereof, or a variant thereof. In some embodiments, the kit comprises a GSH-containing solution or a GSH powder.

In some embodiments, the kit contains multiple packages for any one of the components. In some embodiments, the kit contains a single package for a component of the kit. In some embodiments, the first component and the second component are packaged separately in the kit. In some embodiments, the first component, the second component, and the third component are each packaged separately in the kit. In other embodiments, at least part of the first component and part of the second component are combined in the kit. For example, in some embodiments, the kit comprises a solution that contains a portion of the stabilized DNase I polypeptide comprising one or more non-standard amino acids, a functional fragment thereof, or a variant thereof and a portion of the GSH. In some embodiments, the first component and the second component are combined in a single package in the kit. In some embodiments, all the components are combined in a single package in the kit.

In some embodiments, a single package in the kit is to be administered as a single dose. In some embodiments, a single package in the kit is divided into 2, 3, 4, 5, 6, 7 or more doses. In further embodiments, multiple packages in the kit are to be administered as a single dose. In some embodiments, the first component, the second component, and the third component are each independently to be administered by inhalation, by oral administration, or by injection. In some embodiments, the first component, the second component, the third component, or a combination thereof is suitable to be administered by inhalation. In certain embodiments, the GSH and the stabilized DNase I polypeptide comprising one or more non-standard amino acids, a functional fragment thereof, or a variant thereof are suitable to be administered by inhalation, and the one or more antibiotics are suitable to be administered orally or by injection.

The above disclosure generally describes the present invention. All references disclosed herein are expressly incorporated by reference. A more complete understanding can be obtained by reference to the following specific examples which are provided herein for purposes of illustration only, and are not intended to limit the scope of the invention.

### EXAMPLES

### Example 1: Expression of Seleno-DNase in Genomically Recoded Organisms

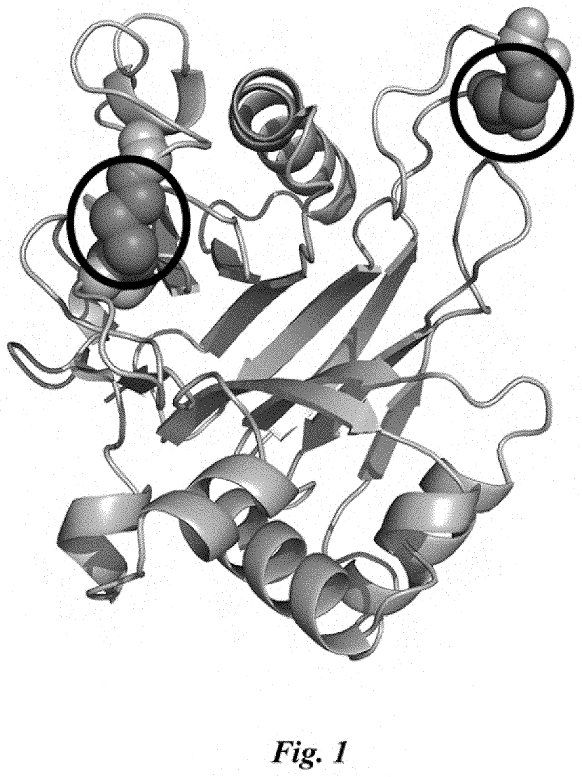

A DNase gene was fused to an MBP in an expression plasmid with a 6×His tag and a Strep-Tag II tag. The 4 cysteine codons of the DNase gene that correspond to the cysteines participating in the disulfide bonds were replaced with UAG codons. FIG. 1 shows the protein structure of DNase. The spheres on the structure indicate the locations of the cysteines that form disulfide bonds, with the center of the circled spheres indicating the two atoms participating in the disulfide bond and to be replaced with a diselenide bond. The expression plasmid was transformed into a GRO E. coli strain that translates the UAG codons to selenocysteine amino acids that form diselenide bonds. GRO E. coli cells were grown at 37° C. to OD600 (optical density at 600 nm) of 0.5. Once the culture reached an OD600 of 0.5, the temperature was lowered to 25° C., and the expression of DNase was induced. After 18 hours of expression the cells were lysed via sonication. The lysate was clarified by centrifugation. Sample purity was assessed using SDS-PAGE (sodium dodecyl sulfate—polyacrylamide gel electrophoresis). Seleno-DNase was tandem purified first by passage through a nickel gravity column and then passaged through a gravity column containing Strep-Tactin XT resin. Purity was assessed to be >95%.

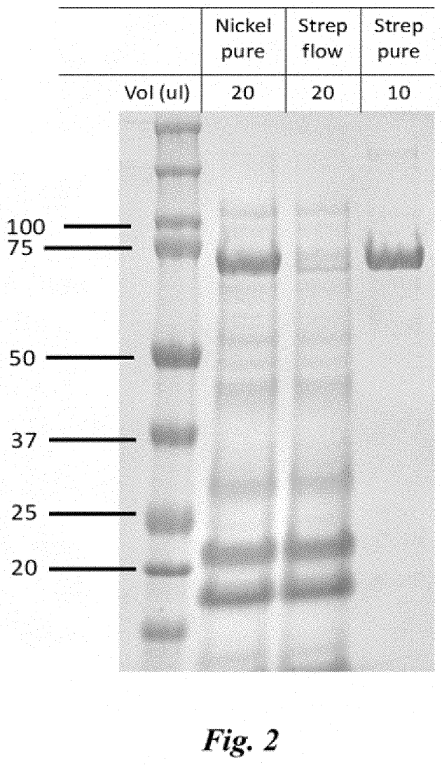

FIG. 2 shows a stained SDS-PAGE gel used to assess purity. The left most lane contains the ladder with the respective sizes of the band denoted in kilodaltons (kDa). The second lane from the left labeled “Nickel pure” shows the proteins in the elutant after just the nickel column. The third lane from the left labeled “Strep flow” shows the proteins in the flow through solution from the Strep-Tactin XT column (which contains the proteins that did not bind to the Strep-Tactin XT column). The rightmost lane labeled “Strep pure” shows the proteins in the elutant of the Strep-Tactin XT column. The resulting purification using a nickel column followed by a Strep-Tactin XT column yields pure MBP-DNase fusion demonstrated by the single band at around 75 kDa.

### Example 2. Activity of Diselenide Substituted DNase at Clinical Concentrations of Glutathione

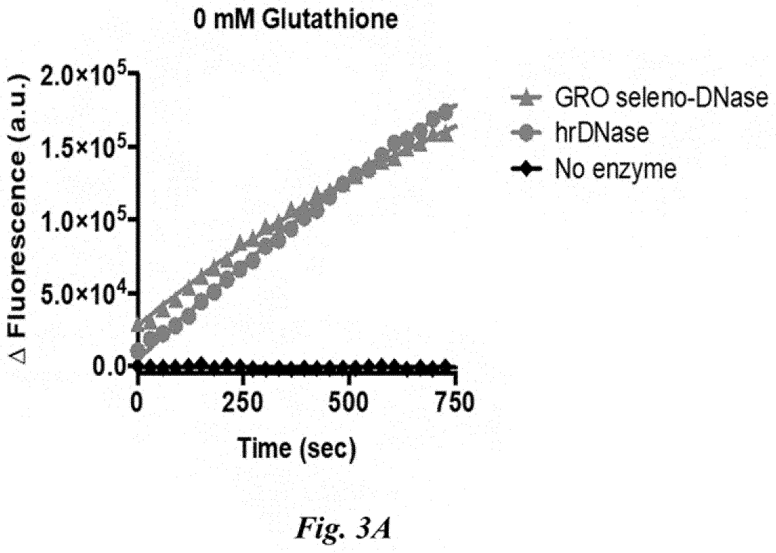

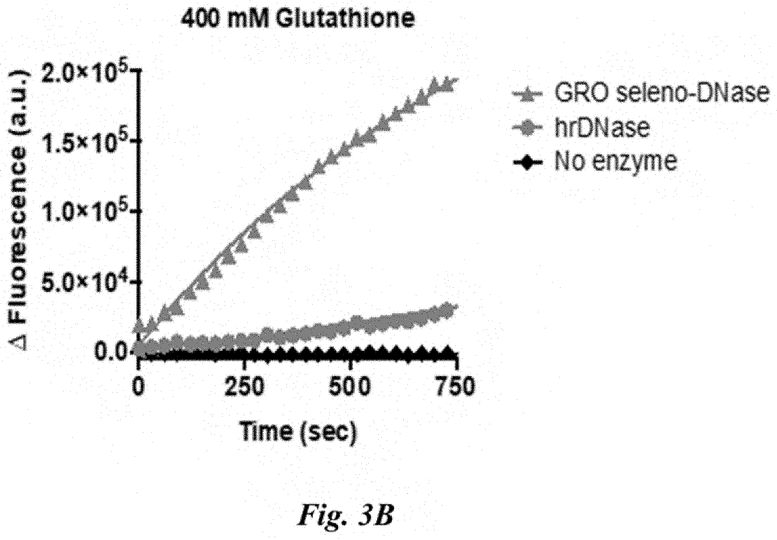

DNase polypetides that contain a diselenide bond in place of a wild type disulfide bond demonstrates activity at higher levels of glutathione. Equivalent units of commercially obtained recombinant human DNase (hrDNase, Prospec Protein Specialists, Ness-Ziona, Israel) and diselenide-substituted DNase (GRO seleno-DNase) were used in a DNase Alert assay (Thermo Fisher Scientific). FIGS. 3A and 3B demonstrate the activity of recombinant human DNase (which does not contain a diselenide bond) versus deselenide substituted DNase. The graph shows the change in fluorescence over time, with an active DNase enzyme demonstrating an increase in the change of fluorescence over time. Cleavage of DNA over time results in a fluorescent signal, and the steepness of the fitted curve reports on enzyme activity. FIG. 3A shows the activity of the enzymes at 0 mM glutathione. The hrDNase and GRO seleno-DNase show similar activity at 0 mM glutathione as the curves are similar. However, as shown by FIG. 3B, the activity of GRO seleno-DNase is much higher than the activity of hrDNase at 400 mM glutathione. This indicates that the GRO seleno-DNase remains active in clinical concentrations of glutathione that would normally deactivate hrDNase.

1.-85. (canceled)

86. A method of treating a lung or pulmonary disease or condition in a human subject in need thereof comprising administering to the human subject a pharmaceutical composition comprising a stabilized deoxyribonuclease I (DNase I) polypeptide comprising one or more non-standard amino acids.

87. The method of claim 86, wherein the method further comprises administering one or more antibiotics to the human subject.

88. The method of claim 86, wherein the method further comprises administering glutathione (GSH) to the human subject.

89. The method of claim 86, wherein the pharmaceutical composition is administered via inhalation.

90. The method of claim 89 wherein the stabilized DNase polypeptide is present in the pharmaceutical composition at a concentration of from 0.1 mg/mL to 10 mg/mL.

91. The method of claim 90, wherein administering comprises administering 0.1 mL to 20 mL of the pharmaceutical composition.

92. The method of claim 87, wherein the one or more antibiotics is selected from the group of classes consisting of Penicillins, Tetracyclines, Cephalosporins, Quinolones, Lincomycins, Macrolides, Sulfonamides, Glycopeptides, Aminoglycosides and Carbapenems.

93. The method of claim 87, wherein the one or more antibiotics comprise amoxicillin, doxycycline, cephalexin, ciprofloxacin, clindamycin, metronidazole, azithromycin, sulfamethoxazole/trimethoprim, amoxicillin/clavulanate, levofloxacin, cefepime, tazobactam/piperacillin, meropenem, amikacin, gentamicin, aztreonam, colistin, or tobramycin.

94. The method of claim 86, wherein the lung or pulmonary disease or condition is an acute or chronic bronchopulmonary disease, atelectasis due to tracheal or bronchial impaction, a complication of a tracheostomy, infectious pneumonia, bronchitis, tracheobronchitis, bronchiectasis, cystic fibrosis (CF), asthma, tuberculosis (TB), or a fungal infection.

95. The method of claim 94, the lung or pulmonary disease or condition is CF.

96. The method of claim 86, wherein the stabilized DNase I polypeptide has a melting temperature (Tm) that is at least 5° C. higher than a Tm of a corresponding DNase I polypeptide that does not comprise the one or more non-standard amino acids.

97. The method of claim 86, wherein the one or more non-standard amino acids comprises selenocysteine.

98. The method of claim 97, wherein the one or more non-standard amino acids comprises two non-standard amino acids that are directly linked by a diselenide bond.

99. The method of claim 97, wherein the selenocysteine is directly linked to a cysteine by a selenyl-sulfhydryl bond.

100. The method of claim 86, wherein the stabilized DNase I polypeptide comprises a sequence with at least 80% sequence identity to at least 225 contiguous amino acids of SEQ ID NO: 1.

101. The method of claim 86, wherein the stabilized DNase I polypeptide has at least 80%, sequence identity to SEQ ID NO:1, SEQ ID NO:2 or SEQ ID NO:3.

102. The method of claim 101, wherein the one or more non-standard amino acids is at:
(a) position 123 of SEQ ID NO:1 or 2;
(b) position 126 of SEQ ID NO:1 or 2;
(c) position 195 of SEQ ID NO:1 or 2, or position 187 of SEQ ID NO: 3; or
(d) position 231 of SEQ ID NO:1 or 2, or position 224 of SEQ ID NO: 3.

103. The method of claim 102, wherein (i) a non-standard amino acid at position 123 of SEQ ID NO:1 or 2 is directly linked by a bond to a non-standard amino acid at position 126 of SEQ ID NO:1 or 2 or (ii) a non-standard amino acid at position 195 of SEQ ID NO:1 or 2 is directly linked by a bond to a non-standard amino acid at position 231 of SEQ ID NO:1 or 2.

104. The method of claim 102, wherein a non-standard amino acid at position 187 of SEQ ID NO:3 is directly linked by a bond to a non-standard amino acid at position 224 of SEQ ID NO:3.

105. A pharmaceutical composition comprising:
(a) a stabilized DNase I polypeptide comprising one or more non-standard amino acids; and
(b) glutathione (GSH).
